(19) United States
(12) Patent Application Publication
(10) Pub. No.: US 2024/0041785 A1
(43) Pub. Date: Feb. 8, 2024

(54) COMPOSITIONS AND METHODS FOR STABILIZATION OF LIPID NANOPARTICLE MRNA VACCINES

(71) Applicant: BioNTech SE, Mainz (DE)

(72) Inventors: Steffen Panzner, Halle (DE); Christian Reinsch, Halle (DE); Kaushik Thanki, Wiesbaden (DE); Sukrut Somani, Mainz (DE); Serguei A. Tchessalov, Billerica, MA (US); Bakul Subodh Bhatnagar, North Reading, MA (US); Ramin Darvari, Lexington, MA (US); Sumit Luthra, Westford, MA (US)

(21) Appl. No.: 18/036,679

(22) PCT Filed: Nov. 14, 2021
(86) PCT No.: PCT/EP21/81674

§ 371 Date: May 12, 2023

Related U.S. Application Data
(60) Provisional application No. 63/115,588, filed on Nov. 18, 2020.
(60) Provisional application No. 63/114,478, filed on Nov. 16, 2020.

(51) Int. Cl. A61K 9/51; A61K 47/24; A61K 47/18; A61K 47/28; A61K 47/26; A61K 39/215

(52) U.S. Cl. CPC A61K 9/5123; A61K 47/24; A61K 47/18; A61K 47/28; A61K 9/5146; A61K 47/26; A61K 9/5192; A61K 39/215; A61K 2039/6018

(57) ABSTRACT

The present disclosure provides technologies relating to stabilization of lipid nanoparticle mRNA compositions (e.g., vaccines).

### BACKGROUND

Messenger RNA (mRNA) is proving to be an exciting therapeutic modality and has garnered significant recent attention, particularly in the vaccine space.

### SUMMARY

The present disclosure provides technologies relating to formulation of RNA (e.g., mRNA) therapeutics, and particular to lipid nanoparticle (LNP) formulations comprising RNA (e.g., mRNA) payloads. Among other things, the present disclosure provides therapeutic RNA formulations (i.e., LNP formulations) that are amenable (e.g., stable) to storage and/or handling at temperatures above about −80° C., or even above about −70° C., about −60° C., about −50° C., about −40° C., about −30° C., or about −20° C. In some embodiments, provided formulations may be amenable to storage and/or handling at temperatures above freezing (e.g., above about 0° C.), at standard refrigeration temperature (e.g., within a range of about 1° C. to about 8° C., or about 2° C. to about 8° C., or about 2° C. to about 6° C., or about 2° C. to about 4° C.), and/or at room temperature (e.g., within a range of about 15° C. to about 25° C., or about 20° C. to about 23° C.).

In some embodiments, the present disclosure provides formulations that are amendable to drying and/or that are dry (e.g., that are lyophilized formulations).

The present disclosure particularly provides certain formulations useful as (and/or in the preparation of) vaccines.

In some embodiments, the present disclosure provides formulations (and specifically LNP formulations) of RNA encoding a viral antigen (e.g., a SARS-CoV2 antigen such as an S-protein or epitope thereof). Specific exemplified formulations include an RNA construct that is a BNT162 construct (e.g., as described in Walsh, E. et al. RNA-Based COVID-19 Vaccine BNT162b2 Selected for a Pivotal Efficacy Study. medRxiv (2020)), e.g., BNT162b2; and PCT application Number No. PCT/EP2020/081981 filed Nov. 12, 2020 and entitled “Coronavirus Vaccine”, the contents of each of which are incorporated herein by reference for purposes described herein.)

In one aspect, a formulation provided herein comprises: (a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; (b) sucrose at a concentration of about 10% w/v in the formulation; and (c) Tris buffer, wherein the Tris buffer is substantially free of sodium chloride and is at a concentration of about 10 mM in the formulation.

In some embodiments, a formulation provided herein is a frozen formulation comprising: (a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; (b) sucrose at a concentration of about 10% w/v in the formulation; and (c) Tris buffer, wherein the Tris buffer is substantially free of sodium chloride and is at a concentration of about 10 mM in the formulation.

In some embodiments, a formulation provided herein is a dry formulation comprising: (a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; (b) sucrose at a concentration of about 10% w/v in the formulation before drying; and (c) Tris buffer, wherein the Tris buffer is substantially free of sodium chloride and is at a concentration of about 10 mM in the formulation before drying.

Methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) Tris buffer, wherein the Tris buffer is substantially free of sodium chloride and is at a concentration of about 10 mM in the formulation; and
   ii) sucrose at a concentration of about 10% w/v in the formulation.

In some embodiments, a method comprising a step of:
administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 10% w/v;
c) Tris buffer, wherein the Tris buffer is substantially free of sodium chloride and is at a concentration of about 10 mM in the formulation;
wherein the formulation is diluted into the dosage form prior to administration.

In one aspect, provided herein is a formulation comprising: (a) a lipid nanoparticle (LNP), wherein the LNP comprises: (i) a payload that is or comprises one or more mRNAs; (ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; (b) trehalose at a concentration of about 10% w/v in the formulation; and (c) Tris buffer, wherein the Tris buffer is substantially free of sodium chloride and is at a concentration of about 10 mM in the formulation.

In some embodiments, a formulation provided herein is a frozen formulation comprising: (a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; (b) trehalose at a concentration of about 10% w/v in the formulation; and c) Tris buffer, wherein the Tris buffer is substantially free of sodium chloride and is at a concentration of about 10 mM in the formulation.

In some embodiments, a formulation provided herein is a dry formulation comprising: (a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; (b) trehalose at a concentration of about 10% w/v in the formulation before drying; and (c) Tris buffer, wherein the Tris buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) Tris buffer, wherein the Tris buffer is substantially free of sodium chloride and is at a concentration of about 10 mM in the formulation; and
   ii) trehalose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) trehalose at a concentration of about 10% w/v;
c) Tris buffer, wherein the Tris buffer is substantially free of sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) Tris buffer, wherein the Tris buffer is substantially free of sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) Tris buffer, wherein the Tris buffer is substantially free of sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation before drying; c) trehalose at a concentration of about 5% w/v in the formulation before drying; d) Tris buffer, wherein the Tris buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
(a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) Tris buffer, wherein the Tris buffer is substantially free of sodium chloride and is at a concentration of about 10 mM in the formulation;
   ii) sucrose at a concentration of about 5% w/v in the formulation; and
   iii) trehalose at a concentration of about 5% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 5% w/v in the formulation;
c) trehalose at a concentration of about 5% w/v in the formulation;
d) Tris buffer, wherein the Tris buffer is substantially free of sodium chloride and is at a concentration of about 10 mM in the formulation; wherein the formulation is diluted into the dosage form prior to administration.

In one aspect, a formulation provided herein comprises: (a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) Tris buffer, wherein the Tris buffer comprises about 6 mg/ml sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) Tris buffer, wherein the Tris buffer comprises about 6 mg/ml sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation before drying; c) Tris buffer, wherein the Tris buffer comprises about 6 mg/ml sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) Tris buffer, wherein the Tris buffer comprises about 6 mg/ml sodium chloride and is at a concentration of about 10 mM in the formulation; and
ii) sucrose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 10% w/v in the formulation;
c) Tris buffer, wherein the Tris buffer comprises about 6 mg/ml sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation; c) Tris buffer, wherein the Tris buffer comprises about 6 mg/ml sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation; c) Tris buffer, wherein the Tris buffer comprises about 6 mg/ml sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation before drying; c) Tris buffer, wherein the Tris buffer comprises about 6 mg/ml sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) Tris buffer, wherein the Tris buffer comprises about 6 mg/ml sodium chloride and is at a concentration of about 10 mM in the formulation; and
   ii) trehalose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) trehalose at a concentration of about 10% w/v in the formulation;
c) Tris buffer, wherein the Tris buffer comprises about 6 mg/ml sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) Tris buffer, wherein the Tris buffer comprises about 6 mg/ml sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) Tris buffer, wherein the Tris buffer comprises about 6 mg/ml sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation before drying; c) trehalose at a concentration of about 5% w/v in the formulation before drying; d) Tris buffer, wherein the Tris buffer comprises about 6 mg/ml sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) Tris buffer, wherein the Tris buffer comprises about 6 mg/ml sodium chloride and is at a concentration of about 10 mM in the formulation;
   ii) sucrose at a concentration of about 5% w/v in the formulation; and
   iii) trehalose at a concentration of about 5% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 5% w/v in the formulation;
c) trehalose at a concentration of about 5% w/v in the formulation;
d) Tris buffer, wherein the Tris buffer comprises about 6 mg/ml sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) His buffer, wherein the His buffer is substantially free of sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) His buffer, wherein the His buffer is substantially free of sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation before drying; c) His buffer, wherein the His buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) His buffer, wherein the His buffer is substantially free of sodium chloride and is at a concentration of about 10 mM in the formulation; and
   ii) sucrose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 10% w/v in the formulation;
d) His buffer, wherein the His buffer is substantially free of sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation; c) His buffer, wherein the His buffer is substantially free of sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation; c) His buffer, wherein the His buffer is substantially free of sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation before drying; c) His buffer, wherein the His buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) His buffer, wherein the His buffer is substantially free of sodium chloride and is at a concentration of about 10 mM in the formulation; and
   ii) trehalose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) trehalose at a concentration of about 10% w/v in the formulation;
d) His buffer, wherein the His buffer is substantially free of sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) His buffer, wherein the His buffer is substantially free of sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) His buffer, wherein the His buffer is substantially free of sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation before drying; c) trehalose at a concentration of about 5% w/v in the formulation before drying; d) His buffer, wherein the His buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) His buffer, wherein the His buffer is substantially free of sodium chloride and is at a concentration of about 10 mM in the formulation;
   ii) sucrose at a concentration of about 5% w/v in the formulation; and
   iii) trehalose at a concentration of about 5% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 5% w/v in the formulation;
c) trehalose at a concentration of about 5% w/v in the formulation;
d) His buffer, wherein the His buffer is substantially free of sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) HEPES buffer, wherein the HEPES buffer is substantially free of sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) HEPES buffer, wherein the HEPES buffer is substantially free of sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation before drying; c) HEPES buffer, wherein the HEPES buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) HEPES buffer, wherein the HEPES buffer is substantially free of sodium chloride and is at a concentration of about 10 mM in the formulation; and
   ii) sucrose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 10% w/v in the formulation;
d) HEPES buffer, wherein the HEPES buffer is substantially free of sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation; c) HEPES buffer, wherein the HEPES buffer is substantially free of sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation; c) HEPES buffer, wherein the HEPES buffer is substantially free of sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) trehalose at a concentration of about 10% w/v in the formulation before drying; c) HEPES buffer, wherein the HEPES buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) HEPES buffer, wherein the HEPES buffer is substantially free of sodium chloride and is at a concentration of about 10 mM in the formulation; and
   ii) trehalose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) trehalose at a concentration of about 10% w/v in the formulation;
d) HEPES buffer, wherein the HEPES buffer is substantially free of sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) HEPES buffer, wherein the HEPES buffer is substantially free of sodium chloride and is at a concentration of about 10 mM in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation; c) trehalose at a concentration of about 5% w/v in the formulation; d) HEPES buffer, wherein the HEPES buffer is substantially free of sodium chloride and is at a concentration of about 10 mM in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 5% w/v in the formulation before drying; c) trehalose at a concentration of about 5% w/v in the formulation before drying; d) HEPES buffer, wherein the HEPES buffer is substantially free of sodium chloride and is at a concentration of about 10 mM in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) HEPES buffer, wherein the HEPES buffer is substantially free of sodium chloride and is at a concentration of about 10 mM in the formulation;
   ii) sucrose at a concentration of about 5% w/v in the formulation; and
   iii) trehalose at a concentration of about 5% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 5% w/v in the formulation;
c) trehalose at a concentration of about 5% w/v in the formulation;
d) HEPES buffer, wherein the HEPES buffer is substantially free of sodium chloride and is at a concentration of about 10 mM in the formulation; wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) PBS buffer, wherein the PBS buffer is substantially free of sodium chloride.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) PBS buffer, wherein the PBS buffer is substantially free of sodium chloride.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation before drying; c) PBS buffer, wherein the PBS buffer is substantially free of sodium chloride.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) PBS buffer, wherein the PBS buffer is substantially free of sodium chloride; and
   ii) sucrose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/ml;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 10% w/v;
c) PBS buffer, wherein the PBS buffer is substantially free of sodium chloride;
wherein the formulation is diluted into the dosage form prior to administration.

In one aspect, a formulation provided herein comprises: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) PBS buffer, wherein the PBS buffer comprises about 6 mg/ml sodium chloride in the formulation.

In some embodiments, a formulation provided herein is a frozen formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation; c) PBS buffer, wherein the PBS buffer comprises about 6 mg/ml sodium chloride in the formulation.

In some embodiments, a formulation provided herein is a dry formulation comprising: a) a lipid nanoparticle (LNP), wherein the LNP comprises: i) a payload that is or comprises one or more mRNAs; ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and b) sucrose at a concentration of about 10% w/v in the formulation before drying; c) PBS buffer, wherein the PBS buffer comprises about 6 mg/ml sodium chloride in the formulation before drying.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) PBS buffer, wherein the PBS buffer comprises about 6 mg/ml sodium chloride in the formulation; and
   ii) sucrose at a concentration of about 10% w/v in the formulation.

In some embodiments, provided herein is a method comprising a step of administering a dosage form of a formulation, wherein the formulation comprises:
a) a lipid nanoparticle (LNP), wherein the LNP comprises:
   i) mRNA at a concentration of about 0.5 mg/mL;
   ii) ((4-hydroxybutyl)azanediyl)bis(hexane-6,1-diyl)bis(2-hexyldecanoate) (ALC-0315) at a concentration of about 7.17 mg/ml;
   iii) 2-[(polyethylene glycol)-2000]-N,N-ditetradecylacetamide (ALC-0159) at a concentration of about 0.89 mg/ml;
   iv) distearoylphosphatidylcholine (DSPC) at a concentration of about 1.56 mg/ml;
   v) cholesterol at a concentration of about 3.1 mg/ml;
b) sucrose at a concentration of about 10% w/v in the formulation;
c) PBS buffer, wherein the PBS buffer comprises about 6 mg/ml sodium chloride;
wherein the formulation is diluted into the dosage form prior to administration.

In some embodiments, methods of providing such formulations described herein are also described herein. In some embodiments, provided herein is a method of preparing a formulation comprising steps of:
a) preparing a lipid nanoparticle (LNP) in a first buffer system, wherein the LNP comprises:
   i) a payload that is or comprises one or more mRNAs;
   ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
   i) PBS buffer, wherein the PBS buffer comprises about 6 mg/ml sodium chloride in the formulation; and
   ii) sucrose at a concentration of about 10% w/v in the formulation wherein the first buffer system comprises sucrose at a concentration of about 10% w/v.

In some embodiments, provided herein is a method of delivering a nucleic acid into a cell in a subject comprising a step of administering a formulation as described in any of the preceding claims.

In some embodiments, provided herein is a method of inducing an immune response in a subject comprising a step of administering to the subject a formulation as described in any of the preceding claims.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Administration: As used herein, the term “administration” refers to the administration of a composition to a subject. Exemplary routes of administration may include bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal. In many embodiments, provided technologies relate to LNP compositions (e.g., comprising a BNT162 construct) that are administered by intramuscular injection. In some embodiments, LNP compositions are administered in a first administration followed by one or more administrations (e.g., one or more booster administrations). In some embodiments, a period of time, e.g., about 24, 48, 72, 96 hours or more, including for about 1, 2, 3, 4, or more weeks, separates each administration of an LNP compositions, e.g., between a first administration and a second administration. In some embodiments, a period of time separating administrations is about 3 weeks (e.g., about 21 days).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antigen: The term “antigen”, as used herein, refers to an agent or moiety that elicits an immune response; and/or that is specifically bound by an antibody or to a T cell receptor (e.g., when presented by an MHC molecule). In some embodiments, an antigen elicits a humoral response (e.g., which may involve or include production of antigen-specific antibodies); in some embodiments, an antigen elicits a cellular response (e.g., which may involve or include T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer), etc. In some embodiments, an antigen is or comprises a polypeptide or epitope thereof. In some embodiments, an antigen is a recombinant antigen.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Combination therapy: As used herein, the term “combination therapy”, or reference to agents being administered “in combination”, refers to those situations in which a subject is simultaneously exposed to two or more therapeutic regimens (e.g., two or more therapeutic agents or modalities). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Expression: As used herein, “expression” of a nucleic acid sequence refers to one or more of the following events: (1) templated synthesis of a complementary nucleic acid (e.g., production of an RNA template from a DNA sequence, for example by transcription); (2) processing of an RNA transcript (e.g., by splicing, editing, 5′ cap formation, and/or 3′ end formation), e.g., to produce an mRNA; (3) translation of an RNA (e.g., an mRNA) into a polypeptide or protein; and/or (4) post-translational modification of a polypeptide or protein. Those skilled in the art will appreciate that, in some circumstances, “expression” may comprise multiple steps of templated synthesis (e.g., reverse transcription of an RNA to generate a DNA strand, followed by transcription of such DNA strand and/or optionally synthesis of complementary DNA strand, for example so as to generate a double-stranded DNA).

Formulation: A “formulation” is a composition prepared and/or provided as described herein. In many embodiments, the term “formulation” is used to refer to LNP compositions—i.e., which comprise an RNA (especially a therapeutic RNA such as an mRNA) and lipids as recited herein.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer (e.g., in contiguous association). In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%. 90%, 95%, 96%, 97%, 98%, 99% or more of the monomeric units (e.g., residues) found in the whole polymer. The whole material or entity may in some embodiments be referred to as the “parent” of the fragment.

Functional: As used herein, the term “functional” is used to refer to a form or fragment of an entity that exhibits a particular property and/or activity. In some embodiments, the property and/or activity of such “functional” fragment is comparable to a its whole.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent identity between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. Representative algorithms and computer programs useful in determining the percent identity between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined for example using the GAP program in the GCG software package using an NWSgapdna.CMP matrix.

Nucleic Acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine. C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or a polypeptide; in some embodiments, such nucleotide sequence may be codon optimized for expression in a particular host (e.g., in a recipient subject). In some embodiments, a nucleic acid that includes a coding sequence also includes one or more introns. In some embodiments, a nucleic acid that includes a coding sequence does not include introns. In some embodiments, nucleic acids are prepared by one or more of: isolation from a natural source, enzymatic synthesis by polymerization based on a complementary template (in some embodiments in vivo; in some embodiments in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Stable: The term “stable,” when applied to compositions herein, means that the compositions maintain one or more aspects of their physical structure and/or activity over a period of time under a designated set of conditions. In some embodiments, the period of time is at least about 1, 2, 3, 4, 5, 6, 7, 8, 9, 10, 11, 12, weeks or more, including for about 1, 2, 3, 4, 5, 6, 7, 8, 9, 10, 11, 12 months or more; in some embodiments, the designated set of conditions is or comprises a temperature above a low temperature threshold. In some embodiments, a low temperature threshold is above about −80° C., −70° C., −50° C., −30° C., −20° C., 0° C., 2° C., 4° C., 8° C., 15°, 20° C., 30° C., 40° C. or higher. In some embodiments, a composition is considered to be stable based on maintenance of colloidal content comprising lipid nanoparticles (LNPs). In some embodiments, a composition is considered to be stable based on maintenance of one or more of LNP characteristics (including, e.g., but not limited to its Z-average and/or polydispersity index (PDI)). In some embodiments, a composition is considered to be stable based on maintenance of nucleic acid integrity, degree (e.g., percent) of nucleic acid encapsulation, and/or nucleic acid expressibility (e.g., level of expression of an encoded polypeptide, as may be expressed for example as percent of a relevant reference level). In some embodiments, compositions described herein are considered stable if lipid nanoparticles within such compositions exhibit less than about 20 nm change in Z-average (including, e.g., less than 19 nm, 18 nm, 17 nm, 16 nm, 15 nm, 14 nm, 13 nm, 12 nm, 11 nm, or less change in Z-average) over a certain period of time under a designated set of conditions compared to a relevant reference level. In some embodiments, compositions described herein are considered stable if lipid nanoparticles within such compositions exhibit less than about 10 nm change in Z-average (including, e.g., less than 9 nm, 8 nm, 7 nm, 6 nm, 5 nm, 4 nm, 3 nm, 2 nm, 1 nm, 0.5 nm, or less change in Z-average) over a certain period of time under a designated set of conditions compared to a relevant reference level. In some embodiments, compositions described herein are considered stable if at least 50% (including e.g., at least 60%, at least 70%, at least 80%, at least 90%, at least 95%, at least 98%, at least 99%, or more) nucleic acid encapsulation is maintained in such compositions over a certain period of time under a designated set of conditions compared to a relevant reference level. In some embodiments, compositions described herein are considered stable if at least 50% (including e.g., at least 60%, at least 70%, at least 80%, at least 90%, at least 95%, at least 98%, at least 99%, or more) of expression level of an encoded polypeptide is maintained over a certain period of time under a designated set of conditions compared to a relevant reference level.

Subject: As used herein, the term “subject,” or “patient,” refers to any organism to which a provided composition is or may be administered, e.g., for experimental, diagnostic, prophylactic, cosmetic, and/or therapeutic purposes. Typical subjects include animals (e.g., mammals such as mice, rats, rabbits, non-human primates, and/or humans). In some embodiments, a subject is a human. In some embodiments, a subject is suffering from or susceptible to one or more disorders or conditions. In some embodiments, a subject displays one or more symptoms of a disorder or condition. In some embodiments, a patient has been diagnosed with one or more disorders or conditions. In some embodiments, a subject is at risk for viral infection, or diseases or disorders associated with viral infection.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Variant: As used herein, in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. In some embodiments, a variant is a distinct molecule that shares one or more such characteristic structural elements but differs in at least one aspect from the reference molecule.

To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions.

In some embodiments, typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. In some embodiments, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

In some embodiments, a “variant” of an amino acid sequence (peptide, protein or polypeptide) may be or comprise an amino acid insertion variant, an amino acid addition (i.e., terminal addition) variant, an amino acid deletion variant and/or an amino acid substitution variant.

In some embodiments, a “variant” may be or comprise a mutants, splice variants, post-translationally modified variants, conformations, isoforms, allelic variants, species variants, and species homologs, in particular those which are naturally occurring. In some embodiments, the term “variant” includes, in particular, fragments of an amino acid sequence.

In some embodiments, an amino acid insertion variant differs from a relevant reference polypeptide by insertion of a single, or of two or more, amino acid(s)

In some embodiments, an amino acid addition variant may comprise an amino- and/or carboxy-terminal fusion (i.e., extension) of one or more amino acids, such as 1, 2, 3, 5, 10, 20, 30, 50, or more amino acids.

In some embodiments, an amino acid deletion variant is characterized by removal of one or more amino acids from a sequence, such as by removal of 1, 2, 3, 5, 10, 20, 30, 50, or more amino acids. In some embodiments, a deletion may be of one or more N-terminal amino acids, one or more C-terminal amino acids, one or more internal amino acids, or a combination thereof.

In some embodiments, an amino acid substitution variant is characterized by at least one residue in a sequence being removed and another residue being inserted in its place. In some embodiments, a substitution is of a residue that is not highly conserved among related polypeptides that, e.g., share one or more common motifs (e.g., characteristic sequence elements) and/or functions. In some embodiments, a substitution is a “conservative” substitution in that the original residue and its replacement share one or more structural or functional attributes or properties (e.g., identity and/or type of charge, or absence thereof; hydrophobicity or hydrophilicity of side chain, three dimensional bulk of side chain, linear or branched character of side chain, presence and/or type of heteroatom in side chain, etc). For example, in some embodiments, a substitution is conservative if it involves swapping residues within a family such as: acidic (aspartate, glutamate), basic (lysine, arginine, histidine), non-polar (alanine, valine, leucine, isoleucine, proline, phenylalanine, methionine, tryptophan), uncharged polar (glycine, asparagine, glutamine, cysteine, serine, threonine, tyrosine), aromatic amino (phenylalanine, tryptophan, tyrosine). In some embodiments, conservative amino acid substitutions within the following groups are considered to be conservative substitutions: glycine, alanine; valine, isoleucine, leucine; aspartic acid, glutamic acid; asparagine, glutamine; serine, threonine; lysine, arginine; and phenylalanine, tyrosine.

In some embodiments, a variant may refer a composition (e.g., a buffer) that is identical to that of a reference composition but for a small number of component alterations, e.g., presence or absence of certain components, or differences in concentrations of certain components.

Wild type: As used herein, the term “wild-type” or “WT” or “native” has its art-understood meaning that refers to an entity having a structure and/or activity as found in nature in a “normal” (as contrasted with mutant, diseased, altered, etc) state or context. Those of ordinary skill in the art will appreciate that wild-type genes and polypeptides often exist in multiple different forms (e.g., alleles). In many embodiments, as used herein, “wild-type” may refer to an amino acid sequence that is found in nature, including allelic variations. A wild type amino acid sequence, peptide or protein has an amino acid sequence that has not been intentionally modified.

### BRIEF DESCRIPTION OF THE DRAWING

FIG. 1A-1F shows an exemplary workflow for production of certain formulations of the present disclosure. In an exemplary first buffer system, particle forming lipids suspended in an organic solvent (e.g., ethanol), and nucleic acids (e.g., mRNA) suspended in an aqeuous buffer (e.g., citrate buffer), are admixed for a period of time (A) until nucleic acid containing lipid particles (e.g., LNPs) are formed (B). In some embodiments, such nucleic acid containing lipid particles can be concentrated and/or transferred to a second buffer system which comprises a protectant (e.g., sucrose, trehalose, etc.) (C). In some embodiments, lipid particles may then be stored or diluted for use, or dried (e.g., by lyophilization or other drying method) (D), or frozen (E), or frozen after drying (F). In some embodiments, after drying, and/or freezing, lipid particles may be stored and/or thawed and/or diluted for use.

FIG. 2A-2B shows certain exemplary formulations of the present disclosure (A) and certain exemplary cycles designed for formulations of the present disclosure (B).

FIG. 3A-3B show exemplary colloidal stability data at various time points and temperatures for exemplary sucrose and trehalose formulations.

FIG. 4A-4B show exemplary % encapsulation data at various time points and temperatures for exemplary sucrose and trehalose formulations.

FIG. 5 shows an exemplary graph of water content for exemplary sucrose and trehalose formulations.

FIG. 6A-6C show exemplary % expression data at various time points and temperatures for exemplary sucrose and trehalose formulations.

FIG. 7A-7D show exemplary data characterizing exemplary formulations of the present disclosure.

FIG. 8A-8B show exemplary colloidal stability data at various time points and temperatures for exemplary sucrose and trehalose formulations.

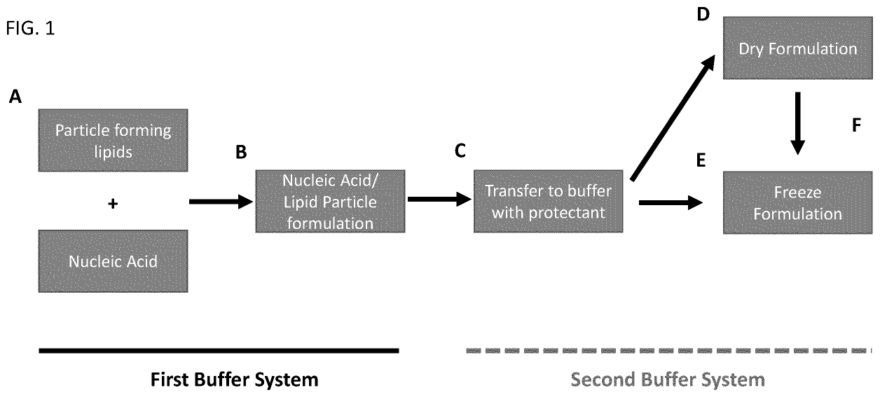

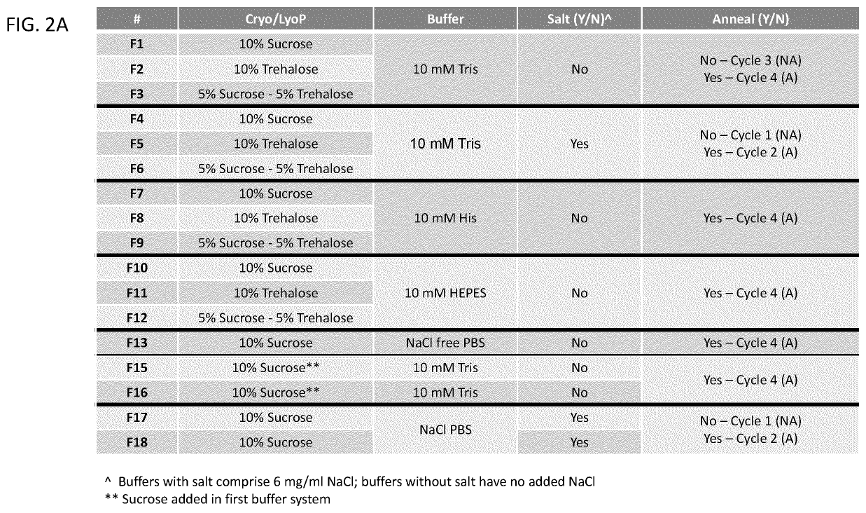

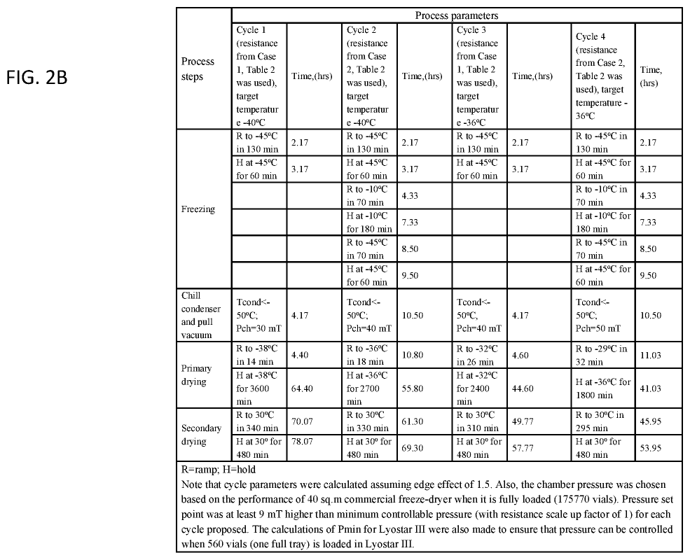

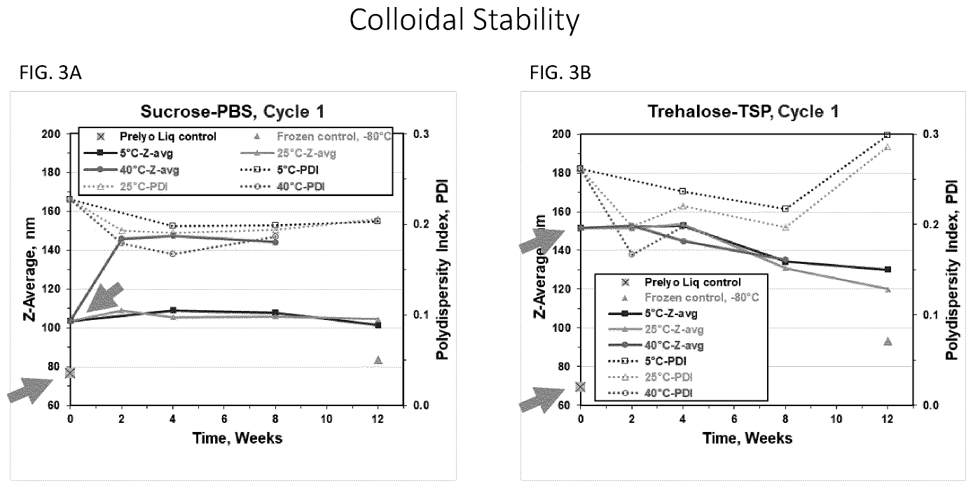

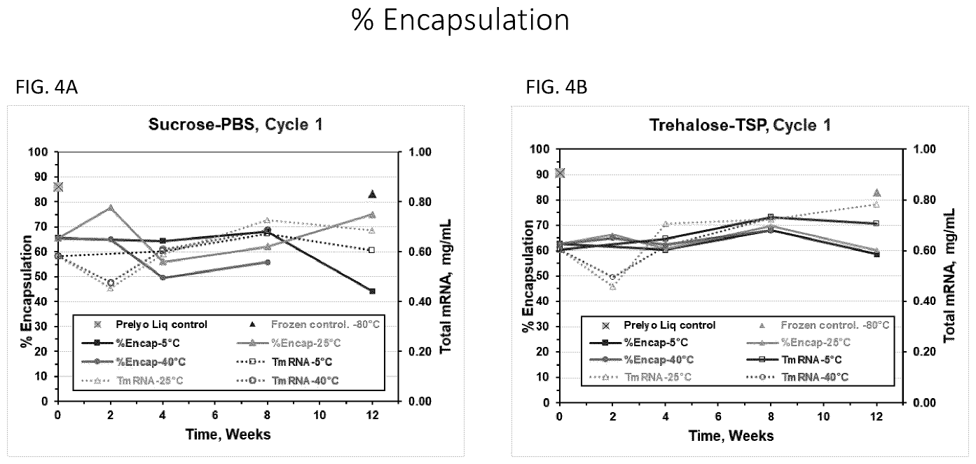

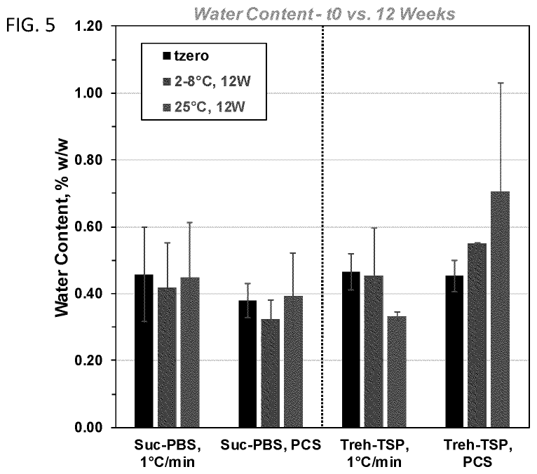

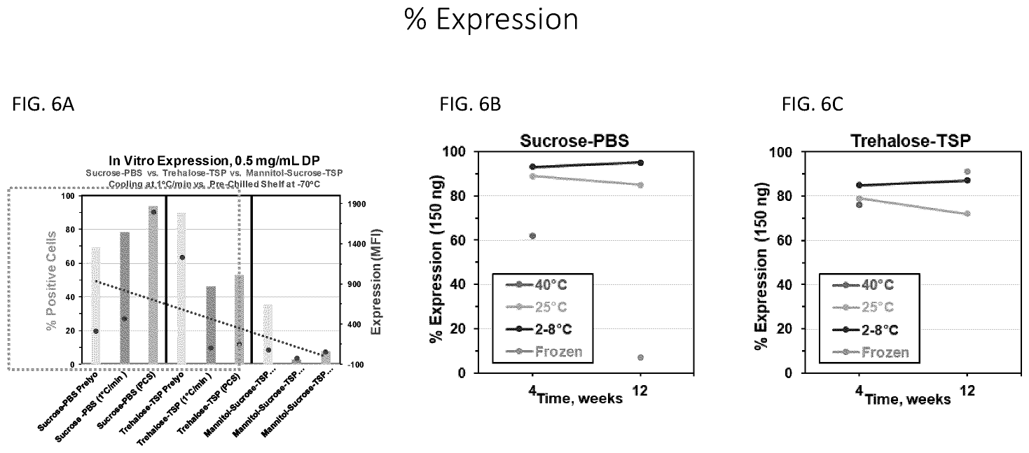

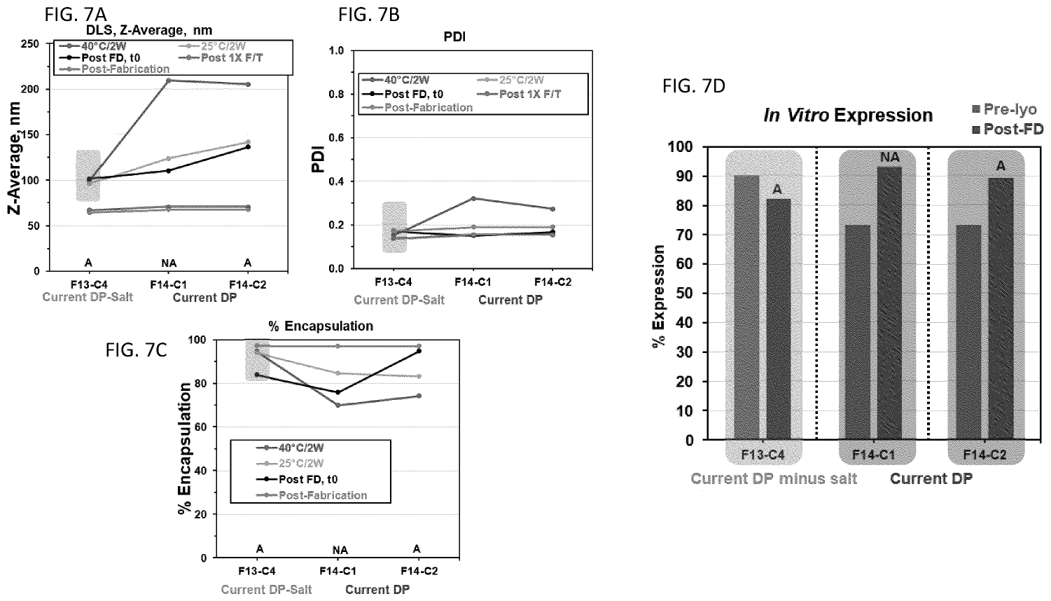

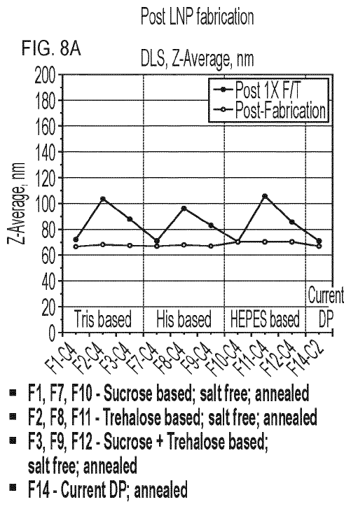

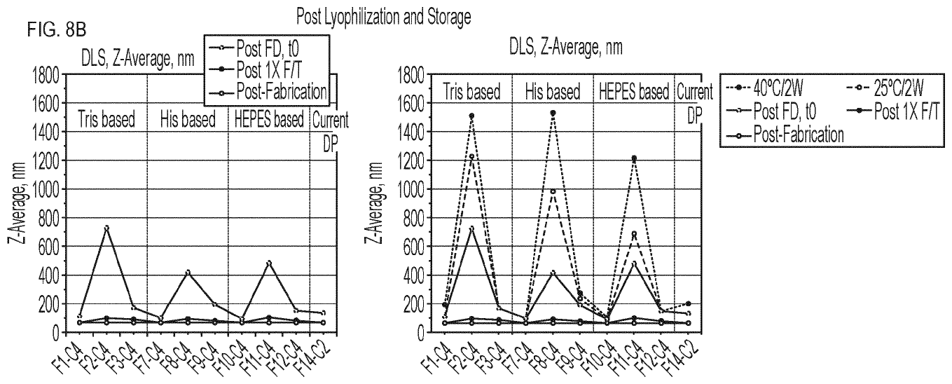

### DETAILED DESCRIPTION OF CERTAIN EMBODIMENTS

The present disclosure provides, among other things, technologies relating to nucleic acid/lipid nanoparticle (LNP) compositions, and particularly RNA/LNP compositions, such as therapeutic RNA/LNP compositions.

Those skilled in the art are aware that one challenge often encountered with nucleic acid/LNP formulations, and particularly with RNA/LNP formulations, is that they require low temperature storage in order to maintain stability over time. Various reports described requirements for temperatures as low as −90° C.; others mention temperatures below −80° C., −70° C., or −60° C. Temperatures as high as −20° C. can often be tolerated for only a short amount of time (e.g., 1, 2, 3, 4 to several days). Temperatures above freezing (e.g., above about 0° C.) and/or achieved by refrigeration (e.g., within a range of about PC to about 8° C., or about 2° C. to about 8° C., or about 2° C. to about 6° C., or about 2° C. to about 4° C.) can often be tolerated only for hours to 1-2 days. Room temperature storage, and particularly long term room temperature storage (e.g., for at least 1-2 days, and desirably for a 1, 2, 3, 4, 5, 6, weeks or more, including for 1, 2, 3, 4, 5, 6, 7, 8, 9, 10, 11, 12 months or more) remains a goal.

In some embodiments, the present disclosure provides nucleic acid/LNP formulations, and particularly RNA/LNP formulations, including particular components (e.g., protectant and/or buffer components), and/or that are prepared according to particular processes, that differ from those of a reference formulation and that modify (e.g., improve) one or more properties relative to that reference formulation. For example, in some embodiments, provided formulations show improvement(s) relative to a reference formulation that comprises the same lipids and nucleic acid, but that differs in protectant and/or buffer, and/or in certain production or processing steps.

In some embodiments, provided technologies achieve preparation of compositions that are dry formulations, or that are amenable to (e.g., stable upon) drying.

In some embodiments, provided compositions can be effectively dried using a lyophilization cycle that is shorter than that required to comparably dry a reference formulation, e.g., an otherwise identical formulation produced using a buffer that includes NaCl, e.g., at a concentration within a range of about 5 to 10 mg/ml (e.g., at about 6 mg/ml).

In some embodiments, provided technologies achieve preparation of compositions that are frozen formulations, or that are amenable to (e.g., stable to) freezing.

In some embodiments, provided technologies achieve preparation of compositions that are stable to storage for at least a specified period of time at temperatures above a low temperature threshold. In some embodiments, the specified period of time may be at least about 1, 2, 3, 4, 5, 6, 7, 8, 9, 10, 11, 12, weeks or more, including for about 1, 2, 3, 4, 5, 6, 7, 8, 9, 10, 11, 12 months or more. In some embodiments, the low temperature threshold may be about −80° C., −70° C., −50° C., −30° C., −20° C., 0° C., 2° C., 4° C., 8° C., 15°, 20° C., 30° C., 40° C. or higher.

In some embodiments, provided technologies are useful to deliver a nucleic acid payload to a subject, e.g., by administration of LNPs that comprise the payload encapsulated by lipids as described herein; in some embodiments, the lipids comprise a cationic lipid, a neutral lipid, a polymer conjugated lipid, and a steroid. In some embodiments, LNPs for use in accordance with the present disclosure are formed from ((4-hydroxybutyl)azanediyl)bis(hexane-6,1-diyl)bis(2-hexyldecanoate) (ALC-0315), 2-[(polyethylene glycol)-2000]-N,N-ditetradecylacetamide (ALC-0159), distearoylphosphatidylcholine (DSPC), and cholesterol, and are combined in relative mass ratios within the range of about 8:1:1.5:3 to about 9:1:2:3.5, respectively.

In some embodiments, a nucleic acid payload is or comprises RNA and/or DNA; in some embodiments, a nucleic acid payload may encode a polypeptide product (e.g., a functional polypeptide, for example that may complement or replace an activity that is needed or desired in a subject, or an immunomodulatory polypeptide, for example that may induce or enhance a desired immune response or activity in a subject).

In some embodiments, provided compositions comprise LNPs (i.e., nucleic acid/LNPs), a protectant, and a buffer. In some embodiments, the buffer does not include sodium ions. In some embodiments the buffer does not include a salt. In some embodiments, the buffer is a HEPES buffer, a Tris buffer, or a His buffer as described herein. In some embodiments, the buffer is a phosphate buffered saline variant that is made without NaCl. In some embodiments, the buffer is a PBS variant that has a reduced level of sodium ions relative to a reference PBS that comprises NaCl, KCl, Na₂HPO₄, and KH₂PO₄; in some embodiments, such reference PBS is a “standard” PBS that comprises (or consists of) 137 mM NaCl (i.e., 8 g/L NaCl), 2.7 mM KCl (i.e., 0.2 g/L KCl), 10 mM Na₂HPO₄ (i.e., 1.44 g/L Na₂HPO₄). and 1.8 mm KH₂PO₄ (i.e., 0.24 g/L KH₂PO₄). In some embodiments, a buffer utilized in accordance with the present disclosure is a PBS variant that has a lower level of sodium ions that than found in such reference standard PBS.

In some embodiments, a protectant utilized in accordance with the present disclosure comprises a disaccharide. In some embodiments, a protectant utilized in accordance with the present disclosure is or comprises sucrose and/or trehalose.

In some embodiments, a protectant is or comprises mannitol. In some embodiments, a protectant is substantially free of mannitol.

In some embodiment, the present disclosure provides technologies by which an LNP preparation (i.e., a nucleic acid/LNP preparation, and particularly an RNA/LNP preparation) is generated and then stored, frozen, and/or dried. In some embodiments, a frozen composition is stored. In some embodiments, a dried composition is stored.

In some embodiments, a dried composition is resuspended and then administered to a subject. In some embodiments, a frozen composition is thawed and then administered to a subject. In some embodiments, a composition may be subjected to one or more rounds of freezing and thawing, to one or more rounds of drying and resuspending, and/or to one or more rounds of freezing and thawing and also one or more rounds of drying and resuspending.

In some embodiments, a composition is diluted prior to being administered.

### Nucleic Acid Payloads

The present disclosure provides, among other things, LNP compositions that comprise a nucleic acid payload (i.e., nucleic acid-LNP compositions).

In some embodiments, a nucleic acid payload may comprise or encode a functional nucleic acid such as, for example, an antisense oligonucleotide (e.g., that may promote RNAseH degradation and/or exon skipping, etc), a ribozyme, a gRNA, a miRNA, and shRNA, an siRNA, etc.

In some embodiments, a nucleic acid payload may encode one or more polypeptides (e.g., as described further hereinbelow).

In some embodiments, a nucleic acid payload utilized in accordance with the present disclosure is or comprises one or more natural nucleic acid residues, or entirely natural nucleic acid residues. In some embodiments, a nucleic acid is, comprises, or consists of one or more non-natural nucleic acid residues (i.e., one or more nucleic acid analogs), or is entirely non-natural nucleic acid residues.

In some embodiments, a nucleic acid payload utilized in accordance with the present disclosure includes one or more internucleotide linkages that is not a phosphodiester bond. For example, in some embodiments, a nucleic acid has one or more phosphorothioate and/or 5′-N-phosphoramidite linkages rather than phosphodiester bonds. In some embodiments, a nucleic acid includes some phosphodiester bonds and some non-phosphodiester bonds.

In some embodiments, a nucleic acid is or comprises one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof).

In some embodiments, a nucleic acid comprises one or more modified sugars (e.g., 2′-fluororibose, ribose, 2′-deoxyribose, arabinose, and hexose) as compared with those in natural nucleic acids.

In some embodiments, a nucleic acid is or comprises one or more peptide nucleic acids.

In some embodiments of the present disclosure, nucleic acids are modified with modifications described herein that impart one or more desirable characteristics, e.g., enhanced stability, potency, etc.

RNA Payloads

In some embodiments, a nucleic acid payload for use in accordance with the present disclosure is an RNA (e.g., an mRNA). In some embodiments, an RNA is produced by templated synthesis. In some embodiments, an RNA is produced by enzymatic synthesis, e.g., by in vitro transcription (e.g., from a DNA template). In some embodiments, an RNA is produced by chemical synthesis.

In some embodiments, an RNA is a “replicon RNA” or simply a “replicon,” in particular “self-replicating RNA” or “self-amplifying RNA.” In some embodiments, a replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments, an RNA for use in accordance with the present disclosure may include one or more modified nucleosides. In some embodiments, the present disclosure provides RNA comprising a modified nucleoside in place of at least one uridine. In some embodiments, modified nucleosides are in place of all uridines in an RNA. In some embodiments, modified nucleosides replacing at least one uridine include, but are not limited to, pseudouridine (ψ), N1-methyl-pseudouridine (m1ψ), and 5-methyl-uridine (m5U), or combinations thereof. In some embodiments, a modified nucleoside replacing at least one, e.g., all, uridine in an RNA may be any one or more of: 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), I-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), I-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, an RNA for use in accordance with the present disclosure comprises a 5′-cap. In some embodiments, an RNA of the present disclosure does not have uncapped 5′-triphosphates. In some embodiments, an RNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In some embodiments, such a guanosine is methylated at the 7-position. Providing an RNA with a 5′-cap, or 5′-cap analog, may be achieved by in vitro transcription, in which a 5′-cap, or 5′-cap analog, is co-transcriptionally expressed into an RNA strand, or may be attached to RNA post-transcriptionally using capping enzymes. In some embodiments, a 5′-cap for RNA is m₂^{7′3-O} Gppp (m₁^{2′-O})ApG (also sometimes referred to as m₂^{7,3′O}G (5′)ppp(5′)m^{2′-O}ApG. In some embodiments, a 5′-cap for RNA of the present disclosure is an analog anti-reverse cap (ARCA Cap (m₂^{7,3′O}G (5′)ppp(5′)G)). In some embodiments, a 5′-cap is Beta-S-ARCA (m₂^{7,2′O}G (5′)ppSp (5′)G). In some embodiments, a 5′-cap is beta-S-ARCA (D1) (m₂^{7,2′-O} GppSpG), or m₂^{7,3′-O}Gppp (m₁^{2′-O})ApG.

In some embodiments, an RNA for use in accordance with the present disclosure comprises a 5′-UTR and/or a 3′-UTR. The term “untranslated region” or “UTR” may relate to a region in a DNA molecule which is transcribed but is not translated into an amino acid sequence, or to the corresponding region in an RNA molecule, such as an mRNA molecule. An UTR can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” preferably does not include a poly(A) sequence. Thus, a 3′-UTR is upstream of a poly(A) sequence (if present), e.g. directly adjacent to a poly(A) sequence.

As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase. It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

In different embodiments, a poly(A) sequence may be of different lengths. In some embodiments, a poly(A) sequence comprises, essentially consists of, or consists of at least 20, at least 30, at least 40, at least 80, or at least 100 A nucleotides. In some embodiments, a poly(A) sequence comprises, essentially consists of, or consists of up to 500, up to 400, up to 300, up to 200, or up to 150 A nucleotides. In some embodiments, a poly(A) sequence comprises about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette. In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, a poly(A) sequence contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, a poly(A) sequence may comprise at least 20, at least 30, at least 40, at least 80, or at least 100 and up to 500, up to 400, up to 300, up to 200, or up to 150 nucleotides. In some embodiments, a poly(A) sequence may essentially consist of at least 20, at least 30, at least 40, at least 80, or at least 100 and up to 500, up to 400, up to 300, up to 200, or up to 150 nucleotides. In some embodiments, a poly(A) sequence may consist of at least 20, at least 30, at least 40, at least 80, or at least 100 and up to 500, up to 400, up to 300, up to 200, or up to 150 nucleotides. In some embodiments, a poly(A) sequence comprises at least 100 nucleotides. In some embodiments, a poly(A) sequence comprises about 150 nucleotides. In some embodiments, a poly(A) sequence comprises about 120 nucleotides.

In some embodiments, a nucleic acid for use in accordance with the present disclosure are codon-optimized and/or guanosine/cytosine (G/C) content is increased compared to wild type coding sequence. This also includes embodiments, wherein one or more sequence regions of a coding sequence are codon-optimized and/or increased in G/C content compared to corresponding sequence regions of a wild type coding sequence. In some embodiments, codon-optimization and/or increase in G/C content does not change the sequence of a encoded amino acid sequence.

G/C Content

In some embodiments of the disclosure, the G/C content of a coding region (e.g., of an RNA) described herein is increased compared to G/C content of a corresponding WT coding sequence, wherein an encoded amino acid sequence is not modified compared to such corresponding WT sequence. In some embodiments, an increase in G/C content may increase translation efficiency of an RNA including such increased G/C content. Those skilled in the art are aware that sequences having an increased G/C content have been reported to be more stable than sequences having an increased adenosine/uracil (A/U) content.

In respect to the fact that several codons code for one and the same amino acid (so-called degeneration of the genetic code), the most favorable codons for stability can be determined (so-called alternative codon usage). Depending on desired amino acid to be encoded by an RNA, there are various possibilities for modification of said RNA sequence, compared to its wild type sequence. In particular, codons which contain A and/or U nucleotides can be modified by substituting these codons by other codons, which code for the same amino acids but contain no A and/or U, or contain a lower content of A and/or U nucleotides.

In various embodiments, the G/C content of a coding region of an RNA utilized in accordance with the present disclosure is increased by at least 10%, at least 20%, at least 30%, at least 40%, at least 50%, at least 55%, or even more compared to G/C content of a coding region of a wild type RNA.

Encoded Polypeptides

As noted herein, in some embodiments, a nucleic acid payload (e.g., an RNA) encodes a polypeptide.

In some embodiments, an encoded polypeptide is or comprises an antibody agent, or a polypeptide chain or functional fragment thereof. In some embodiments, an antibody agent is or comprises a single chain antibody agent such as an scFC, a camelid antibody, etc.

In some embodiments, an encoded polypeptide is or comprises a cytokine, a growth factor, an apoptotic factor, a differentiation-inducing factor, a cell-surface receptor, a ligand, a hormone, etc.

In some embodiments, an encoded polypeptide is an enzyme.

In some embodiments, an encoded polypeptide is a regulatory polypeptide such as a transcription factor, a chaperone, etc.

In some embodiments, an encoded polypeptide is or comprises a polypeptide whose expression replaces or activates an activity that is reduced or lacking in a subject.

In some embodiments, an encoded polypeptide is or comprises a polypeptide that induces and/or enhances an immune response in a subject. In some embodiments, an encoded polypeptide is or comprises at least one epitope that is specifically bound by an immunoglobulin agent (e.g., an antibody and/or a T cell receptor, etc).

In some embodiments, an encoded polypeptide is or comprises an antigen (or epitope thereof). In some embodiments, an antigen may be characteristic of a particular disease, disorder or condition. For example, an antigen may be or comprise a tumor antigen (e.g., a neoantigen) and/or an antigen associated with an infectious agent (e.g., a virus or microbe such as a bacterium or fungus). In some embodiments, an antigen associated with an infectious agent may be an antigen that is displayed on a surface of such infectious agent and/or may mediate infection by such agent (e.g., by participating in interaction with a receptor on recipient cells).

In some embodiments, an antigen may be or comprise a viral antigen, e.g. an antigen associated with a virus selected from the group consisting of: adenovirus, cytomegalovirus, herpes virus, human papillomavirus, measles virus, rubella virus, coronavirus, respiratory syncytial virus, influenza virus, and mumps virus. In some embodiments, an antigen may be or comprise a viral antigen associated with a virus selected from a Class I, Class II, Class III, Class IV, Class V, Class VI, or Class VII virus, based on the Baltimore classification system. In some embodiments, an antigen may be or comprise a viral antigen associated with a virus selected from viral family Adenoviridae, Papovaviridae, Parvoviridae, Herpesviridae, Poxviridae, Anelloviridae, Pleolipoviridae, Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae, Arteriviridae, and Hepeviridae. In some particular embodiments, a viral antigen may be a coronaviral antigen.

In some particular embodiments, a viral antigen may be an antigen derived from a SARS-CoV-2 protein sequence (e.g., may be or comprise such sequence, a fragment thereof, or a variant of either). In some embodiments, the present disclosure provides a polypeptide with an antigen sequence derived from a SARS-CoV-2 S protein sequence. In some embodiments, a polypeptide is or comprises an antigen sequence derived from a Receptor Binding Domain (RBD) of SARS-COV-2 S protein sequence.

In some embodiments, a payload as described herein is associated, or encapsulated within the lipid portion of a LNP. In some embodiments, a payload as described herein is associated within a lipid portion of the LNP. In some embodiments, a payload as described here is encapsulated within a lipid portion of the LNP. In some embodiments, association with (e.g., encapsulation within) such lipid portion reduces susceptibility of a payload degradation (e.g., enzymatic degradation), for example over a given period of time and/or under particular conditions.

According to some embodiments, a signal peptide is fused, either directly or through a linker, to an antigenic peptide or protein. In some embodiments, signal peptides for use in accordance with the present disclosure are sequences, which typically exhibit a length of about 15 to about 30 amino acids and may be located at an N-terminus of an antigenic peptide or protein, without being limited thereto. In some embodiments, signal peptides as defined herein allow the transport of an antigenic peptide or protein as encoded by an RNA into a defined cellular compartment, e.g., a cell surface, endoplasmic reticulum (ER) or endosomal-lysosomal compartment.

A signal peptide sequence as may be utilized in accordance with certain embodiments of the present disclosure may be or comprise, for example, a signal peptide sequence of an immunoglobulin, e.g., a signal peptide sequence of an immunoglobulin heavy chain variable region, wherein an immunoglobulin may be human immunoglobulin.

Signal peptides for use in accordance with the present disclosure are used in order to promote secretion of an encoded antigenic peptide or protein. In some embodiments, a signal peptide as defined herein is fused to an encoded antigenic peptide or protein as defined herein. In some embodiments, an RNA described herein comprises at least one coding region encoding an antigenic peptide or protein and a signal peptide, where said signal peptide are fused to an antigenic peptide or protein, e.g., to an N-terminus of an antigenic peptide or protein as described herein.

In some embodiments, a trimerization domain is fused, either directly or through a linker, e.g., a glycine/serine linker, to an antigenic peptide or protein. In some embodiments, a trimerization domain is fused, either directly or through a linker, e.g., a glycine/serine linker, to an antigenic peptide or protein, which is also fused to a signal peptide as described herein.

In some embodiments, such trimerization domains are located at a C-terminus of an antigenic peptide or protein, without being limited thereto. Trimerization domains as defined herein allow trimerization of an antigenic peptide or protein as encoded by RNA. Examples of trimerization domains as defined herein include, without being limited thereto, foldon, a natural trimerization domain of T4 fibritin. A C-terminal domain of T4 fibritin (foldon) is obligatory for the formation of a fibritin trimer structure and can be used as an artificial trimerization domain.

In some embodiments, a transmembrane domain is fused, either directly or through a linker, e.g., a glycine/serine linker, to an antigenic peptide or protein. Accordingly, in some embodiments, a transmembrane domain is fused, either directly or through a linker, e.g., a glycine/serine linker, to an antigenic peptide or protein, which is also fused to a signal peptide and/or trimerization domain as described herein).

In many embodiments, a transmembrane domains utilized in accordance with the present disclosure is located at a C-terminus of an antigenic peptide or protein, without being limited thereto. In some embodiments, such transmembrane domains are located at a C-terminus of a trimerization domain, if present, without being limited thereto. In some embodiments, a trimerization domain is present between a SARS-CoV-2 S protein, a variant thereof, or a fragment thereof, i.e., an antigenic peptide or protein, and a transmembrane domain.

In some embodiments, a transmembrane domain utilized in accordance with the present disclosure may allow the anchoring into a cellular membrane of an antigenic peptide or protein as encoded by an RNA.

Coronavirus

Coronaviruses are enveloped, positive-sense, single-stranded RNA ((+) ssRNA) viruses. They have the largest genomes (26-32 kb) among known RNA viruses and are phylogenetically divided into four genera (α, β, γ, and δ), with betacoronaviruses further subdivided into four lineages (A, B, C, and D). Coronaviruses infect a wide range of avian and mammalian species, including humans. Some human coronaviruses generally cause mild respiratory diseases, although severity can be greater in infants, the elderly, and the immunocompromised. Middle East respiratory syndrome coronavirus (MERS-CoV) and severe acute respiratory syndrome coronavirus (SARS-CoV), belonging to betacoronavirus lineages C and B, respectively, are highly pathogenic. Both viruses emerged into the human population from animal reservoirs within the last 15 years and caused outbreaks with high case-fatality rates. The outbreak of severe acute respiratory syndrome coronavirus-2 (SARS-CoV-2) that causes atypical pneumonia (coronavirus disease 2019; COVID-19) has raged in China since mid-December 2019, and has developed to be a public health emergency of international concern. SARS-CoV-2 (MN908947.3) belongs to betacoronavirus lineage B. It has at least 70% sequence similarity to SARS-CoV.

In general, coronaviruses have four structural proteins, namely, envelope (E), membrane (M), nucleocapsid (N), and spike (S). The E and M proteins have important functions in the viral assembly, and the N protein is necessary for viral RNA synthesis. The critical glycoprotein S is responsible for virus binding and entry into target cells. The S protein is synthesized as a single-chain inactive precursor that is cleaved by furin-like host proteases in the producing cell into two noncovalently associated subunits, S1 and S2. The S1 subunit contains the receptor-binding domain (RBD), which recognizes the host-cell receptor. The S2 subunit contains the fusion peptide, two heptad repeats, and a transmembrane domain, all of which are required to mediate fusion of the viral and host-cell membranes by undergoing a large conformational rearrangement. The S1 and S2 subunits trimerize to form a large prefusion spike.

The S precursor protein of SARS-CoV-2 can be proteolytically cleaved into S1 (685 aa) and S2 (588 aa) subunits. The S1 subunit consists of the receptor-binding domain (RBD), which mediates virus entry into sensitive cells through the host angiotensin-converting enzyme 2 (ACE2) receptor.

SARS-CoV-2 coronavirus full length spike (S) protein consist of 1273 amino acids (see SEQ ID NO: 1).

In some embodiments, the present disclosure utilizes RNA encoding a peptide or protein comprising at least an epitope SARS-CoV-2 S protein for inducing an immune response against coronavirus S protein, in particular SARS-CoV-2 S protein in a subject. In some embodiments, RNA of the present disclosure encodes an amino acid sequence comprising SARS-CoV-2 S protein, an immunogenic fragment of SARS-CoV-2 S protein, or immunogenic variants thereof.

In some embodiments, full length spike (S) protein according to SEQ ID NO: 1 is modified in such a way that the prototypical prefusion conformation is stabilized. Stabilization of the prefusion conformation may be obtained by introducing two consecutive proline substitutions at AS residues 986 and 987 in the full length spike protein. Specifically, spike (S) protein stabilized protein variants are obtained in a way that the amino acid residue at position 986 is exchanged to proline and the amino acid residue at position 987 is also exchanged to proline. In some embodiments, a SARS-CoV-2 S protein variant comprises the amino acid sequence shown in SEQ ID NO: 7.

In some embodiments, the vaccine antigen described herein comprises, consists essentially of or consists of a spike protein (S) of SARS-CoV-2, a variant thereof, or a fragment thereof.

In some embodiments, RNA encoding a vaccine antigen (i) comprises the nucleotide sequence of nucleotides 49 to 3819 of SEQ ID NO: 2, 8 or 9, a nucleotide sequence having at least 99%, 98%, 97%, 96%, 95%, 90%, 85%, or 80% identity to the nucleotide sequence of nucleotides 49 to 3819 of SEQ ID NO: 2, 8 or 9, or a fragment of the nucleotide sequence of nucleotides 49 to 3819 of SEQ ID NO: 2, 8 or 9, or the nucleotide sequence having at least 99%, 98%, 97%, 96%, 95%, 90%, 85%, or 80% identity to the nucleotide sequence of nucleotides 49 to 3819 of SEQ ID NO: 2, 8 or 9; and/or (ii) encodes an amino acid sequence comprising the amino acid sequence of amino acids 17 to 1273 of SEQ ID NO: 1 or 7, an amino acid sequence having at least 99%, 98%, 97%, 96%, 95%, 90%, 85%, or 80% identity to the amino acid sequence of amino acids 17 to 1273 of SEQ ID NO: 1 or 7, or an immunogenic fragment of the amino acid sequence of amino acids 17 to 1273 of SEQ ID NO: 1 or 7, or the amino acid sequence having at least 99%, 98%, 97%, 96%, 95%, 90%, 85%, or 80% identity to the amino acid sequence of amino acids 17 to 1273 of SEQ ID NO: 1 or 7. In some embodiments, RNA encoding a vaccine antigen (i) comprises the nucleotide sequence of nucleotides 49 to 3819 of SEQ ID NO: 2, 8 or 9; and/or (ii) encodes an amino acid sequence comprising the amino acid sequence of amino acids 17 to 1273 of SEQ ID NO: 1 or 7.

In some embodiments, a vaccine antigen comprises, consists essentially of, or consists of SARS-CoV-2 spike S1 fragment (S1) (the S1 subunit of a spike protein (S) of SARS-CoV-2), a variant thereof, or a fragment thereof.

In some embodiments, RNA encoding a vaccine antigen (i) comprises the nucleotide sequence of nucleotides 49 to 2049 of SEQ ID NO: 2, 8 or 9, a nucleotide sequence having at least 99%, 98%, 97%, 96%, 95%, 90%, 85%, or 80% identity to the nucleotide sequence of nucleotides 49 to 2049 of SEQ ID NO: 2, 8 or 9, or a fragment of the nucleotide sequence of nucleotides 49 to 2049 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2049 of SEQ ID NO: 2, 8 or 9; and/or (ii) encodes an amino acid sequence comprising the amino acid sequence of amino acids 17 to 683 of SEQ ID NO: 1, an amino acid sequence having at least 99%, 98%, 97%, 96%, 95%, 90%, 85%, or 80% identity to the amino acid sequence of amino acids 17 to 683 of SEQ ID NO: 1, or an immunogenic fragment of the amino acid sequence of amino acids 17 to 683 of SEQ ID NO: 1, or the amino acid sequence having at least 99%, 98%, 97%, 96%, 95%, 90%, 85%, or 80% identity to the amino acid sequence of amino acids 17 to 683 of SEQ ID NO: 1. In some embodiments, RNA encoding a vaccine antigen (i) comprises the nucleotide sequence of nucleotides 49 to 2049 of SEQ ID NO: 2, 8 or 9; and/or (ii) encodes an amino acid sequence comprising the amino acid sequence of amino acids 17 to 683 of SEQ ID NO: 1.

In some embodiments, RNA encoding a vaccine antigen (i) comprises the nucleotide sequence of nucleotides 49 to 2055 of SEQ ID NO: 2, 8 or 9, a nucleotide sequence having at least 99%, 98%, 97%, 96%, 95%, 90%, 85%, or 80% identity to the nucleotide sequence of nucleotides 49 to 2055 of SEQ ID NO: 2, 8 or 9, or a fragment of the nucleotide sequence of nucleotides 49 to 2055 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2055 of SEQ ID NO: 2, 8 or 9; and/or (ii) encodes an amino acid sequence comprising the amino acid sequence of amino acids 17 to 685 of SEQ ID NO: 1, an amino acid sequence having at least 99%, 98%, 97%, 96%, 95%, 90%, 85%, or 80% identity to the amino acid sequence of amino acids 17 to 685 of SEQ ID NO: 1, or an immunogenic fragment of the amino acid sequence of amino acids 17 to 685 of SEQ ID NO: 1, or the amino acid sequence having at least 99%, 98%, 97%, 96%, 95%, 90%, 85%, or 80% identity to the amino acid sequence of amino acids 17 to 685 of SEQ ID NO: 1. In some embodiments, RNA encoding a vaccine antigen (i) comprises the nucleotide sequence of nucleotides 49 to 2055 of SEQ ID NO: 2, 8 or 9; and/or (ii) encodes an amino acid sequence comprising the amino acid sequence of amino acids 17 to 685 of SEQ ID NO: 1.

In some embodiments, a vaccine antigen comprises, consists essentially of, or consists of receptor binding domain (RBD) of the S1 subunit of a spike protein (S) of SARS-CoV-2, a variant thereof, or a fragment thereof. The amino acid sequence of amino acids 327 to 528 of SEQ ID NO: 1, a variant thereof, or a fragment thereof is also referred to herein as “RBD” or “RBD domain”.

In some embodiments, RNA encoding a vaccine antigen (i) comprises the nucleotide sequence of nucleotides 979 to 1584 of SEQ ID NO: 2, 8 or 9, a nucleotide sequence having at least 99%, 98%, 97%, 96%, 95%, 90%, 85%, or 80% identity to the nucleotide sequence of nucleotides 979 to 1584 of SEQ ID NO: 2, 8 or 9, or a fragment of the nucleotide sequence of nucleotides 979 to 1584 of SEQ ID NO: 2, 8 or 9, or the nucleotide sequence having at least 99%, 98%, 97%, 96%, 95%, 90%, 85%, or 80% identity to the nucleotide sequence of nucleotides 979 to 1584 of SEQ ID NO: 2, 8 or 9; and/or (ii) encodes an amino acid sequence comprising the amino acid sequence of amino acids 327 to 528 of SEQ ID NO: 1, an amino acid sequence having at least 99%, 98%, 97%, 96%, 95%, 90%, 85%, or 80% identity to the amino acid sequence of amino acids 327 to 528 of SEQ ID NO: 1, or an immunogenic fragment of the amino acid sequence of amino acids 327 to 528 of SEQ ID NO: 1, or the amino acid sequence having at least 99%, 98%, 97%, 96%, 95%, 90%, 85%, or 80% identity to the amino acid sequence of amino acids 327 to 528 of SEQ ID NO: 1. In some embodiments, RNA encoding a vaccine antigen (i) comprises the nucleotide sequence of nucleotides 979 to 1584 of SEQ ID NO: 2, 8 or 9; and/or (ii) encodes an amino acid sequence comprising the amino acid sequence of amino acids 327 to 528 of SEQ ID NO: 1.

According to some embodiments, a signal peptide is fused, either directly or through a linker, to a SARS-CoV-2 S protein, a variant thereof, or a fragment thereof, i.e., an antigenic peptide or protein. Accordingly, in some embodiments, a signal peptide is fused to the above described amino acid sequences derived from SARS-CoV-2 S protein or immunogenic fragments thereof (antigenic peptides or proteins) comprised by vaccine antigens described herein.

In some embodiments, signal peptides for use in accordance with the present disclosure are sequences, which typically exhibit a length of about 15 to about 30 amino acids and are located at an N-terminus of an antigenic peptide or protein, without being limited thereto. In some embodiments, signal peptides as defined herein allow the transport of an antigenic peptide or protein as encoded by an RNA into a defined cellular compartment, e.g., a cell surface, endoplasmic reticulum (ER) or an endosomal-lysosomal compartment. In some embodiments, a signal peptide sequence as defined herein includes, without being limited thereto, a signal peptide sequence of SARS-CoV-2 S protein, in particular a sequence comprising the amino acid sequence of amino acids 1 to 16 or 1 to 19 of SEQ ID NO: 1 or a functional variant thereof.

A signal peptide sequence as may be utilized in accordance with certain embodiments of the present disclosure may be or comprise, for example, a signal peptide sequence of an immunoglobulin, e.g., a signal peptide sequence of an immunoglobulin heavy chain variable region, wherein an immunoglobulin may be human immunoglobulin.

Signal peptides for use in accordance with the present disclosure are used in order to promote secretion of an encoded antigenic peptide or protein. In some embodiments, a signal peptide as defined herein is fused to an encoded antigenic peptide or protein as defined herein. In some embodiments, an RNA described herein comprises at least one coding region encoding an antigenic peptide or protein and a signal peptide, where said signal peptide is fused to an antigenic peptide or protein, e.g., to an N-terminus of an antigenic peptide or protein as described herein.

In some embodiments, a trimerization domain is fused, either directly or through a linker, e.g., a glycine/serine linker, to a SARS-CoV-2 S protein, a variant thereof, or a fragment thereof, i.e., an antigenic peptide or protein. Accordingly, in some embodiments, a trimerization domain is fused to the above described amino acid sequences derived from SARS-CoV-2 S protein or immunogenic fragments thereof (antigenic peptides or proteins) comprised by vaccine antigens described above (which may optionally be fused to a signal peptide as described herein).

In some embodiments, such trimerization domains are located at a C-terminus of an antigenic peptide or protein, without being limited thereto. Trimerization domains as defined herein allow trimerization of an antigenic peptide or protein as encoded by RNA. Examples of trimerization domains as defined herein include, without being limited thereto, foldon, a natural trimerization domain of T4 fibritin. A C-terminal domain of T4 fibritin (foldon) is obligatory for the formation of a fibritin trimer structure and can be used as an artificial trimerization domain.

In some embodiments, a transmembrane domain is fused, either directly or through a linker, e.g., a glycine/serine linker, to a SARS-CoV-2 S protein, a variant thereof, or a fragment thereof, i.e., an antigenic peptide or protein. Accordingly, in some embodiments, a transmembrane domain is fused to the above described amino acid sequences derived from SARS-CoV-2 S protein or immunogenic fragments thereof (antigenic peptides or proteins) comprised by vaccine antigens described above (which may optionally be fused to a signal peptide and/or trimerization domain as described herein).

In many embodiments, a transmembrane domains utilized in accordance with the present disclosure is located at a C-terminus of an antigenic peptide or protein, without being limited thereto. In some embodiments, such transmembrane domains are located at a C-terminus of a trimerization domain, if present, without being limited thereto. In some embodiments, a trimerization domain is present between a SARS-CoV-2 S protein, a variant thereof, or a fragment thereof, i.e., an antigenic peptide or protein, and a transmembrane domain.

In some embodiments, a transmembrane domain utilized in accordance with the present disclosure may allow the anchoring into a cellular membrane of an antigenic peptide or protein as encoded by an RNA.

In some embodiments, a transmembrane domain sequence as defined herein includes, without being limited thereto, a transmembrane domain sequence of SARS-CoV-2 S protein, in particular a sequence comprising the amino acid sequence of amino acids 1207 to 1254 of SEQ ID NO: 1, or a functional variant thereof.

As presented herein, trimerization domains are used in order to promote trimerization of an encoded antigenic peptide or protein. In some embodiments, a trimerization domain as defined herein is fused to an antigenic peptide or protein as defined herein. In some embodiments, an RNA described herein comprises at least one coding region encoding an antigenic peptide or protein and a trimerization domain as defined herein, said trimerization domain being fused to an antigenic peptide or protein, e.g., to a C-terminus of an antigenic peptide or protein.

In some embodiments, vaccine antigens described herein comprise a contiguous sequence of SARS-CoV-2 coronavirus spike (S) protein that consists of or essentially consists of the above described amino acid sequences derived from SARS-CoV-2 S protein or immunogenic fragments thereof (antigenic peptides or proteins) comprised by vaccine antigens described herein. In some embodiments, vaccine antigens described herein comprise a contiguous sequence of SARS-CoV-2 coronavirus spike (S) protein of no more than 220 amino acids, 215 amino acids, 210 amino acids, or 205 amino acids.

In some embodiments, an RNA encoding a vaccine antigen is nucleoside modified messenger RNA (modRNA) described herein as BNT162b2 (RBP020.1 or RBP020.2). In some embodiments, an RNA encoding a vaccine antigen is nucleoside modified messenger RNA (modRNA) described herein as RBP020.2.

As described herein, different embodiments of nucleoside modified messenger RNA (modRNA) are as follows:

### BNT162b2; RBP020.1 (SEQ ID NO: 19; SEQ ID NO: 7)

Structure: m27,3′-OGppp (m12′-O)ApG)-hAg-Kozak-S1S2-PP-FI-A30L70
Encoded antigen: Viral spike protein (S1S2 protein) of the SARS-CoV-2 (S1 S2 full-length protein, sequence variant)

### BNT162b2; RBP020.2 (SEQ ID NO: 20; SEQ ID NO: 7)

Structure: m27,3′-OGppp (m12′-O)ApG)-hAg-Kozak-S1 S2-PP-FI-A30L70
Encoded antigen: Viral spike protein (S1S2 protein) of the SARS-CoV-2 (S1S2 full-length protein, sequence variant)

Nucleotide Sequence of RBP020.1

Nucleotide sequence is shown with individual sequence elements as indicated in bold letters. In addition, the sequence of the translated protein is shown in italic letters below the coding nucleotide sequence (*=stop codon).

```
                10 20 30 40 50 53
AGAAUAAACU AGUAUUCUUC UGGUCCCCAC AGACUCAGAG AGAACCCGCC ACC
                                                    hAg-Kozak
                63 73 83 93 103 113
AUGUUUGUGU UUCUUGUGCU GCUGCCUCUU GUGUCUUCUC AGUGUGUGAA UUUGACAACA
    M F V F L V L L P L V S S Q C V N L T T
                                                    S protein
              123 133 143 153 163 173
AGAACACAGC UGCCACCAGC UUAUACAAAU UCUUUUACCA GAGGAGUGUA UUAUCCUGAU
    R T Q L P P A Y T N S F T R G V Y Y P D
                                                    S protein
              183 193 203 213 223 233
AAAGUGUUUA GAUCUUCUGU GCUGCACAGC ACACAGGACC UGUUUCUGCC AUUUUUUAGC
    K V F R S S V L H S T Q D L F L P F F S
                                                    S protein
              243 253 263 273 283 293
AAUGUGACAU GGUUUCAUGC AAUUCAUGUG UCUGGAACAA AUGGAACAAA AAGAUUUGAU
    N V T W F H A I H V S G T N G T K R F D
                                                    S protein
              303 313 323 333 343 353
AAUCCUGUGC UGCCUUUUAA UGAUGGAGUG UAUUUUGCUU CAACAGAAAA GUCAAAUAUU
    N P V L P F N D G V Y F A S T E K S N I
                                                    S protein
              363 373 383 393 403 413
AUUAGAGGAU GGAUUUUUGG AACAACACUG GAUUCUAAAA CACAGUCUCU GCUGAUUGUG
    I R G W I F G T T L D S K T Q S L L I V
                                                    S protein
              423 433 443 453 463 473
AAUAAUGCAA CAAAUGUGGU GAUUAAAGUG UGUGAAUUUC AGUUUUGUAA UGAUCCUUUU
    N N A T N V V I K V C E F Q F C N D P F
                                                    S protein
              483 493 503 513 523 533
CUGGGAGUGU AUUAUCACAA AAAUAAUAAA UCUUGGAUGG AAUCUGAAUU UAGAGUGUAU
    L G V Y Y H K N N K S W M E S E F R V Y
                                                    S protein
              543 553 563 573 583 593
UCCUCUGCAA AUAAUUGUAC AUUUGAAUAU GUGUCUCAGC CUUUUCUGAU GGAUCUGGAA
    S S A N N C T F E Y V S Q P F L M D L E
                                                    S protein
              603 613 622 633 643 653
GGAAAACAGG GCAAUUUUAA AAAUCUGAGA GAAUUUGUGU UUAAAAAUAU UGAUGGAUAU
    G K Q G N F K N L R E F V F K N I D G Y
                                                    S protein
              663 673 683 693 703 713
UUUAAAAUUU AUUCUAAACA CACACCAAUU AAUUUAGUGA GAGAUCUGCC UCAGGGAUUU
    F K I Y S K H T P I N L V R D L P Q G F
                                                    S protein
              723 733 743 753 763 773
UCUGCUCUGG AACCUCUGGU GGAUCUGCCA AUUGGCAUUA AUAUUACAAG AUUUCAGACA
    S A L E P L V D L P I G I N I T R F Q T
                                                    S protein
              783 793 803 813 823 833
CUGCUGGCUC UGCACAGAUC UUAUCUGACA CCUGGAGAUU CUUCUUCUGG AUGGACAGCC
    L L A L H R S Y L T P G D S S S G W T A
                                                    S protein
              843 853 863 873 883 893
GGAGCUGCAG CUUAUUAUGU GGGCUAUCUG CAGCCAAGAA CAUUUCUGCU GAAAUAUAAU
    G A A A Y Y V G Y L Q P R T F L L K Y N
                                                    S protein
              903 913 923 933 943 953
GAAAAUGGAA CAAUUACAGA UGCUGUGGAU UGUGCUCUGG AUCCUCUGUC UGAAACAAAA
    E N G T I T D A V D C A L D P L S E T K
                                                    S protein
              963 973 983 993 1003 1013
UGUACAUUAA AAUCUUUUAC AGUGGAAAAA GGCAUUUAUC AGACAUCUAA UUUUAGAGUG
    C T L K S F T V E K G I Y Q T S N E R V
                                                    S protein
            1023 1033 1043 1053 1063 1073
CAGCCAACAG AAUCUAUUGU GAGAUUUCCA AAUAUUACAA AUCUGUGUCC AUUUGGAGAA
    Q P T E S I V R F P N I T N L C P F G E
                                                    S protein
            1083 1093 1103 1113 1123 1133
GUGUUUAAUG CAACAAGAUU UGCAUCUGUG UAUGCAUGGA AUAGAAAAAG AAUUUCUAAU
    V F N A T R F A S V Y A W N R K R I S N
                                                    S protein
            1143 1153 1163 1173 1183 1193
UGUGUGGCUG AUUAUUCUGU GCUGUAUAAU AGUGCUUCUU UUUCCACAUU UAAAUGUUAU
    C V A D Y S V L Y N S A S F S T F K C Y
                                                    S protein
            1203 1213 1223 1233 1243 1253
GGAGUGUCUC CAACAAAAUU AAAUGAUUUA UGUUUUACAA AUGUGUAUGC UGAUUCUUUU
    G V S P T K L N D L C F T N V Y A D S F
                                                    S protein
            1263 1273 1283 1293 1303 1313
GUGAUCAGAG GUGAUGAAGU GAGACAGAUU GCCCCCGGAC AGACAGGAAA AAUUGCUGAU
    V I R G D E V R Q I A P G Q T G K I A D
                                                    S protein
            1323 1333 1343 1353 1363 1373
UACAAUUACA AACUGCCUGA UGAUUUUACA GGAUGUGUGA UUGCUUGGAA UUCUAAUAAU
    Y N Y K L P D D E T G C V I A W N S N N
                                                    S protein
            1383 1393 1403 1413 1423 1433
UUAGAUUCUA AAGUGGGAGG AAAUUACAAU UAUCUGUACA GACUGUUUAG AAAAUCAAAU
    L D S K V G G N Y N Y L Y R L F R K S N
                                                    S protein
            1443 1453 1463 1473 1483 1493
CUGAAACCUU UUGAAAGAGA UAUUUCAACA GAAAUUUAUC AGGCUGGAUC AACACCUUGU
    L K P F E R D I S T E I Y Q A G S T P C
                                                    S protein
            1503 1513 1523 1533 1543 1553
AAUGGAGUGG AAGGAUUUAA UUGUUAUUUU CCAUUACAGA GCUAUGGAUU UCAGCCAACC
    N G V E G F N C Y F P L Q S Y G F Q P T
                                                    S protein
            1563 1573 1583 1593 1603 1613
AAUGGUGUGG GAUAUCAGCC AUAUAGAGUG GUGGUGCUGU CUUUUGAACU GCUGCAUGCA
    N G V G Y Q P Y R V V V I S F E L L H A
                                                    S protein
            1623 1633 1643 1653 1663 1673
CCUGCAACAG UGUGUGGACC UAAAAAAUCU ACAAAUUUAG UGAAAAAUAA AUGUGUGAAU
    P A T V C G P K K S T A L V K N K C V N
                                                    S protein
            1683 1693 1703 1713 1723 1733
UUUAAUUUUA AUGGAUUAAC AGGAACAGGA GUGCUGACAG AAUCUAAUAA AAAAUUUCUG
    F N F N G L T G T G V L T E S N K K F L
                                                    S protein
            1743 1753 1763 1773 1783 1793
CCUUUUCAGC AGUUUGGCAG AGAUAUUGCA GAUACCACAG AUGCAGUGAG AGAUCCUCAG
    P F Q Q F G R D I A D T T D A V R D P Q
                                                    S protein
            1803 1813 1823 1833 1843 1853
ACAUUAGAAA UUCUGGAUAU UACACCUUGU UCUUUUGGGG GUGUGUCUGU GAUUACACCU
    T L E I L D I T P C S F G G V S V I T P
                                                    S protein
            1863 1873 1883 1893 1903 1913
GGAACAAAUA CAUCUAAUCA GGUGGCUGUG CUGUAUCAGG AUGUGAAUUG UACAGAAGUG
    G T N T S N Q V A V L Y Q D V N C T E V
                                                    S protein
            1923 1933 1943 1953 1963 1973
CCAGUGGCAA UUCAUGCAGA UCAGCUGACA CCAACAUGGA GAGUGUAUUC UACAGGAUCU
    P V A I H A D Q L T P T W R V Y S T G S
                                                    S protein
            1983 1993 2003 2013 2023 2033
AAUGUGUUUC AGACAAGAGC AGGAUGUCUG AUUGGAGCAG AACAUGUGAA UAAUUCUUAU
    N V F Q T R A G C L I G A E H V N N S Y
                                                    S protein
            2043 2053 2063 2073 2083 2093
GAAUGUGAUA UUCCAAUUGG AGCAGGCAUU UGUGCAUCUU AUCAGACACA GACAAAUUCC
    E C D I P I G A G I C A S Y Q T Q T N S
                                                    S protein
            2103 2113 2123 2133 2143 2153
CCAAGGAGAG CAAGAUCUGU GGCAUCUCAG UCUAUUAUUG CAUACACCAU GUCUCUGGGA
    P R R A R S V A S Q S I I A Y T M S L G
                                                    S protein
            2163 2173 2183 2193 2203 2213
GCAGAAAAUU CUGUGGCAUA UUCUAAUAAU UCUAUUGCUA UUCCAACAAA UUUUACCAUU
    A E N S V A Y S N N S I A I P T N F T I
                                                    S protein
            2223 2233 2243 2253 2263 2273
UCUGUGACAA CAGAAAUUUU ACCUGUGUCU AUGACAAAAA CAUCUGUGGA UUGUACCAUG
    S V T T E I L P V S M T K T S V D C T M
                                                    S protein
            2283 2293 2303 2313 2323 2333
UACAUUUGUG GAGAUUCUAC AGAAUGUUCU AAUCUGCUGC UGCAGUAUGG AUCUUUUUGU
    Y I C G D S T E C S N L L L Q Y G S F C
                                                    S protein
            2343 2353 2363 2373 2383 2393
ACACAGCUGA AUAGAGCUUU AACAGGAAUU GCUGUGGAAC AGGAUAAAAA UACACAGGAA
    T Q L N R A L T G I A V E Q D K N T Q E
                                                    S protein
            2403 2413 2423 2433 2443 2453
GUGUUUGCUC AGGUGAAACA GAUUUACAAA ACACCACCAA UUAAAGAUUU UGGAGGAUUU
    V F A Q V K Q I Y K T P P I K D F G G F
                                                    S protein
            2463 2473 2483 2493 2503 2513
AAUUUUAGCC AGAUUCUGCC UGAUCCUUCU AAACCUUCUA AAAGAUCUUU UAUUGAAGAU
    N F S Q I L P D P S K P S K R S F I E D
                                                    S protein
            2523 2533 2543 2553 2563 2573
CUGCUGUUUA AUAAAGUGAC ACUGGCAGAU GCAGGAUUUA UUAAACAGUA UGGAGAUUGC
    L L F N K V T L A D A G F I K Q Y G D C
                                                    S protein
            2583 2593 2603 2613 2623 2633
CUGGGUGAUA UUGCUGCAAG AGAUCUGAUU UGUGCUCAGA AAUUUAAUGG ACUGACAGUG
    L G D I A A R D L I C A Q K E N G L T V
                                                    S protein
            2643 2653 2663 2673 2683 2693
CUGCCUCCUC UGCUGACAGA UGAAAUGAUU GCUCAGUACA CAUCUGCUUU ACUGGCUGGA
    L P P L L T D E M I A Q Y T S A L L A G
                                                    S protein
            2703 2713 2723 2733 2743 2753
ACAAUUACAA GCGGAUGGAC AUUUGGAGCU GGAGCUGCUC UGCAGAUUCC UUUUGCAAUG
    T I T S G W T E G A G A A L Q I P F A M
                                                    S protein
            2763 2773 2783 2793 2803 2813
CAGAUGGCUU ACAGAUUUAA UGGAAUUGGA GUGACACAGA AUGUGUUAUA UGAAAAUCAG
    Q M A Y R F N G I G V T Q N V L Y E N Q
                                                    S protein
            2823 2833 2843 2853 2863 2873
AAACUGAUUG CAAAUCAGUU UAAUUCUGCA AUUGGCAAAA UUCAGGAUUC UCUGUCUUCU
    K L I A N Q F N S A I G K I Q D S L S S
                                                    S protein
            2883 2893 2903 2913 2923 2933
ACAGCUUCUG CUCUGGGAAA ACUGCAGGAU GUGGUGAAUC AGAAUGCACA GGCACUGAAU
    T A S A L G K L Q D V V N Q N A Q A L N
                                                    S protein
            2943 2953 2963 2973 2983 2993
ACUCUGGUGA AACAGCUGUC UAGCAAUUUU GGGGCAAUUU CUUCUGUGCU GAAUGAUAUU
    T L V K Q L S S N F G A I S S V L N D I
                                                    S protein
            3003 3013 3023 3033 3043 3053
CUGUCUAGAC UGGAUCCUCC UGAAGCUGAA GUGCAGAUUG AUAGACUGAU CACAGGAAGA
    L S R L D P P E A E V Q I D R L I T G R
                                                    S protein
            3063 3073 3083 3093 3103 3113
CUGCAGUCUC UGCAGACUUA UGUGACACAG CAGCUGAUUA GAGCUGCUGA AAUUAGAGCU
    L Q S L Q T Y V T Q Q L I R A A E I R A
                                                    S protein
            3123 3133 3143 3153 3163 3173
UCUGCUAAUC UGGCUGCUAC AAAAAUGUCU GAAUGUGUGC UGGGACAGUC AAAAAGAGUG
    S A N L A A T K M S E C V L G Q S K R V
                                                    S protein
            3183 3193 3203 3213 3223 3233
GAUUUUUGUG GAAAAGGAUA UCAUCUGAUG UCUUUUCCAC AGUCUGCUCC ACAUGGAGUG
    D F C G K G Y H L M S F P Q S A P H G V
                                                    S protein
            3243 3253 3263 3273 3283 3293
GUGUUUUUAC AUGUGACAUA UGUGCCAGCA CAGGAAAAGA AUUUUACCAC AGCACCAGCA
    V F L H V T Y V P A Q E K N F T T A P A
                                                    S protein
            3303 3313 3323 3333 3343 3353
AUUUGUCAUG AUGGAAAAGC ACAUUUUCCA AGAGAAGGAG UGUUUGUGUC UAAUGGAACA
    I C H D G K A H F P R E G V F V S N G T
                                                    S protein
            3363 3373 3383 3393 3403 3413
CAUUGGUUUG UGACACAGAG AAAUUUUUAU GAACCUCAGA UUAUUACAAC AGAUAAUACA
    H W F V T Q R N F Y E P Q I I T T D N T
                                                    S protein
            3423 3433 3443 3453 3463 3473
UUUGUGUCAG GAAAUUGUGA UGUGGUGAUU GGAAUUGUGA AUAAUACAGU GUAUGAUCCA
    F V S G N C D V V I G I V N N T V Y D P
                                                    S protein
            3483 3493 3503 3513 3523 3533
CUGCAGCCAG AACUGGAUUC UUUUAAAGAA GAACUGGAUA AAUAUUUUAA AAAUCACACA
    L Q P E L D S F K E E L D K Y F K N H T
                                                    S protein
            3543 3553 3563 3573 3583 3593
UCUCCUGAUG UGGAUUUAGG AGAUAUUUCU GGAAUCAAUG CAUCUGUGGU GAAUAUUCAG
    S P D V D L G D I S G I N A S V V N I Q
                                                    S protein
            3603 3613 3623 3633 3643 3653
AAAGAAAUUG AUAGACUGAA UGAAGUGGCC AAAAAUCUGA AUGAAUCUCU GAUUGAUCUG
    K E I D R L N E V A K N L N E S L I D L
                                                    S protein
            3663 3673 3683 3693 3703 3713
CAGGAACUUG GAAAAUAUGA ACAGUACAUU AAAUGGCCUU GGUACAUUUG GCUUGGAUUU
    Q E L G K Y E Q Y I K W P W Y I W L G F
                                                    S protein
            3723 3733 3743 3753 3763 3773
AUUGCAGGAU UAAUUGCAAU UGUGAUGGUG ACAAUUAUGU UAUGUUGUAU GACAUCAUGU
    I A G L I A I V M V T I M L C C M T S C
                                                    S protein
            3783 3793 3803 3813 3823 3833
UGUUCUUGUU UAAAAGGAUG UUGUUCUUGU GGAAGCUGUU GUAAAUUUGA UGAAGAUGAU
    C S C L K G C C S C G S C C K F D E D D
                                                    S protein
            3843 3853 3863 3873 3878
UCUGAACCUG UGUUAAAAGG AGUGAAAUUG CAUUACACAU GAUGA
    S E P V L K G V K L H Y T * *
                                                    S protein
            3888 3898 3908 3918 3928 3938
CUCGAGCUGG UACUGCAUGC ACGCAAUGCU AGCUGCCCCU UUCCCGUCCU GGGUACCCCG
                                                    FI element
            3948 3958 3968 3978 3988 3998
AGUCUCCCCC GACCUCGGGU CCCAGGUAUG CUCCCACCUC CACCUGCCCC ACUCACCACC
                                                    FI element
            4008 4018 4028 4038 4048 4058
UCUGCUAGUU CCAGACACCU CCCAAGCACG CAGCAAUGCA GCUCAAAACG CUUAGCCUAG
                                                    FI element
            4068 4078 4088 4098 4108 4118
CCACACCCCC ACGGGAAACA GCAGUGAUUA ACCUUUAGCA AUAAACGAAA GUUUAACUAA
                                                    FI element
            4128 4138 4148 4158 4168 4173
GCUAUACUAA CCCCAGGGUU GGUCAAUUUC GUGCCAGCCA CACCCUGGAG CUAGC
                                                    FI element
            4183 4193 4203 4213 4223 4233
AAAAAAAAAA AAAAAAAAAA AAAAAAAAAA GCAUAUGACU AAAAAAAAAA AAAAAAAAAA
                                                    Poly(A)
            4243 4253 4263 4273 4283
AAAAAAAAAA AAAAAAAAAA AAAAAAAAAA AAAAAAAAAA AAAAAAAAAA
                                                    Poly(A)
```

Nucleotide Sequence of RBP020.2 Nucleotide sequence is shown with individual sequence elements as indicated in bold letters. In addition, the sequence of the translated protein is shown in italic letters below the coding nucleotide sequence (*=stop codon).

```
                10 20 30 40 50 53
AGAAUAAACU AGUAUUCUUC UGGUCCCCAC AGACUCAGAG AGAACCCGCC ACC
                                                    hAg-Kozak
                63 73 83 93 103 113
AUGUUCGUGU UCCUGGUGCU GCUGCCUCUG GUGUCCAGCC AGUGUGUGAA CCUGACCACC
    M F V F L V L L P L V S S Q C V N L T T
                                                    S protein
              123 133 143 153 163 173
AGAACACAGC UGCCUCCAGC CUACACCAAC AGCUUUACCA GAGGCGUGUA CUACCCCGAC
    R T Q L P P A Y T N S F T R G V Y Y P D
                                                    S protein
              183 193 203 213 223 233
AAGGUGUUCA GAUCCAGCGU GCUGCACUCU ACCCAGGACC UGUUCCUGCC UUUCUUCAGC
    K V F R S S V L H S T Q D L F L P F F S
                                                    S protein
              243 253 263 273 283 293
AACGUGACCU GGUUCCACGC CAUCCACGUG UCCGGCACCA AUGGCACCAA GAGAUUCGAC
    N V T W F H A I H V S G T N G T K R F D
                                                    S protein
              303 313 323 333 343 353
AACCCCGUGC UGCCCUUCAA CGACGGGGUG UACUUUGCCA GCACCGAGAA GUCCAACAUC
    N P V L P F N D G V Y F A S T E K S N I
                                                    S protein
              363 373 383 393 403 413
AUCAGAGGCU GGAUCUUCGG CACCACACUG GACAGCAAGA CCCAGAGCCU GCUGAUCGUG
    I R G W I F G T T L D S K T Q S L L I V
                                                    S protein
              423 433 443 453 463 473
AACAACGCCA CCAACGUGGU CAUCAAAGUG UGCGAGUUCC AGUUCUGCAA CGACCCCUUC
    N N A T N V V I K V C E F Q F C N D P F
                                                    S protein
              483 493 503 513 523 533
CUGGGCGUCU ACUACCACAA GAACAACAAG AGCUGGAUGG AAAGCGAGUU CCGGGUGUAC
    L G V Y Y H K N N K S W M E S E F R V Y
                                                    S protein
              543 553 563 573 583 593
AGCAGCGCCA ACAACUGCAC CUUCGAGUAC GUGUCCCAGC CUUUCCUGAU GGACCUGGAA
    S S A N N C T F E Y V S Q P F L M D L E
                                                    S protein
              603 613 623 633 643 653
GGCAAGCAGG GCAACUUCAA GAACCUGCGC GAGUUCGUGU UUAAGAACAU CGACGGCUAC
    G K Q G N F K N L R E F V F K N I D G Y
                                                    S protein
              663 673 683 693 703 713
UUCAAGAUCU ACAGCAAGCA CACCCCUAUC AACCUCGUGC GGGAUCUGCC UCAGGGCUUC
    F K I Y S K H T P I N L V R D L P Q G F
                                                    S protein
              723 733 743 753 763 773
UCUGCUCUGG AACCCCUGGU GGAUCUGCCC AUCGGCAUCA ACAUCACCCG GUUUCAGACA
    S A L E P L V D L P I G I N I T R F Q T
                                                    S protein
              783 793 803 813 823 833
CUGCUGGCCC UGCACAGAAG CUACCUGACA CCUGGCGAUA GCAGCAGCGG AUGGACAGCU
    L L A L H R S Y L T P G D S S S G W T A
                                                    S protein
              843 853 863 873 883 893
GGUGCCGCCG CUUACUAUGU GGGCUACCUG CAGCCUAGAA CCUUCCUGCU GAAGUACAAC
    G A A A Y Y V G Y L Q P R T F L L K Y N
                                                    S protein
              903 913 923 933 943 953
GAGAACGGCA CCAUCACCGA CGCCGUGGAU UGUGCUCUGG AUCCUCUGAG CGAGACAAAG
    E N G T I T D A V D C A L D P L S E T K
                                                    S protein
              963 973 983 993 1003 1013
UGCACCCUGA AGUCCUUCAC CGUGGAAAAG GGCAUCUACC AGACCAGCAA CUUCCGGGUG
    C T L K S F T V E K G I Y Q T S N F R V
                                                    S protein
            1023 1033 1043 1053 1063 1073
CAGCCCACCG AAUCCAUCGU GCGGUUCCCC AAUAUCACCA AUCUGUGCCC CUUCGGCGAG
    Q P T E S I V R F P N I T N L C P F G E
                                                    S protein
            1083 1093 1103 1113 1123 1133
GUGUUCAAUG CCACCAGAUU CGCCUCUGUG UACGCCUGGA ACCGGAAGCG GAUCAGCAAU
    V F N A T R F A S V Y A W N R K R I S N
                                                    S protein
            1143 1153 1163 1173 1183 1193
UGCGUGGCCG ACUACUCCGU GCUGUACAAC UCCGCCAGCU UCAGCACCUU CAAGUGCUAC
    C V A D Y S V L Y N S A S F S T F K C Y
                                                    S protein
            1203 1213 1223 1233 1243 1253
GGCGUGUCCC CUACCAAGCU GAACGACCUG UGCUUCACAA ACGUGUACGC CGACAGCUUC
    G V S P T K L N D L C F T N V Y A D S F
                                                    S protein
            1263 1273 1283 1293 1303 1313
GUGAUCCGGG GAGAUGAAGU GCGGCAGAUU GCCCCUGGAC AGACAGGCAA GAUCGCCGAC
    V I R G D E V R Q I A P G Q T G K I A D
                                                    S protein
            1323 1333 1343 1353 1363 1373
UACAACUACA AGCUGCCCGA CGACUUCACC GGCUGUGUGA UUGCCUGGAA CAGCAACAAC
    Y N Y K L P D D F T G C V I A W N S N N
                                                    S protein
            1383 1393 1403 1413 1423 1433
CUGGACUCCA AAGUCGGCGG CAACUACAAU UACCUGUACC GGCUGUUCCG GAAGUCCAAU
    L D S K V G G N Y N Y L Y R L F R K S N
                                                    S protein
            1443 1453 1463 1473 1483 1493
CUGAAGCCCU UCGAGCGGGA CAUCUCCACC GAGAUCUAUC AGGCCGGCAG CACCCCUUGU
    L K P F E R D I S T E I Y Q A G S T P C
                                                    S protein
            1503 1513 1523 1533 1543 1553
AACGGCGUGG AAGGCUUCAA CUGCUACUUC CCACUGCAGU CCUACGGCUU UCAGCCCACA
    N G V E G F N C Y F P L Q S Y G F Q P T
                                                    S protein
            1563 1573 1583 1593 1603 1613
AAUGGCGUGG GCUAUCAGCC CUACAGAGUG GUGGUGCUGA GCUUCGAACU GCUGCAUGCC
    N G V G Y Q P Y R V V V L S F E L L H A
                                                    S protein
            1623 1633 1643 1653 1663 1673
CCUGCCACAG UGUGCGGCCC UAAGAAAAGC ACCAAUCUCG UGAAGAACAA AUGCGUGAAC
    P A T V C G P K K S T N L V K N K C V N
                                                    S protein
            1683 1693 1703 1713 1723 1733
UUCAACUUCA ACGGCCUGAC CGGCACCGGC GUGCUGACAG AGAGCAACAA GAAGUUCCUG
    F N F I N G L T G T G V L T E S N K K L
                                                    S protein
            1743 1753 1763 1773 1783 1793
CCAUUCCAGC AGUUUGGCCG GGAUAUCGCC GAUACCACAG ACGCCGUUAG AGAUCCCCAG
    P F Q Q F G R D I A D T T D A V R D P Q
                                                    S protein
            1803 1813 1823 1833 1843 1853
ACACUGGAAA UCCUGGACAU CACCCCUUGC AGCUUCGGCG GAGUGUCUGU GAUCACCCCU
    T L E I L D I T P C S F G G V S V I T P
                                                    S protein
            1863 1873 1883 1893 1903 1913
GGCACCAACA CCAGCAAUCA GGUGGCAGUG CUGUACCAGG ACGUGAACUG UACCGAAGUG
    G T N T S N Q V A V L Y Q D V N C T E V
                                                    S protein
            1923 1933 1943 1953 1963 1973
CCCGUGGCCA UUCACGCCGA UCAGCUGACA CCUACAUGG GGGUGUACUC CACCGGCAGC
    P V A I H A D Q L T P T W R V Y S T G S
                                                    S protein
            1983 1993 2003 2013 2023 2033
AAUGUGUUUC AGACCAGAGC CGGCUGUCUG AUCGGAGCCG AGCACGUGAA CAAUAGCUAC
    N V F Q T R A G C L I G A E H V N N S Y
                                                    S protein
            2043 2053 2063 2073 2083 2093
GAGUGCGACA UCCCCAUCGG CGCUGGAAUC UGCGCCAGCU ACCAGACACA GACAAACAGC
    E C D I P I G A G I C A S Y Q T Q T N S
                                                    S protein
            2103 2113 2123 2133 2143 2153
CCUCGGAGAG CCAGAAGCGU GGCCAGCCAG AGCAUCAUUG CCUACACAAU GUCUCUGGGC
    P R R A R S V A S Q S I I A Y T M S L G
                                                    S protein
            2163 2173 2183 2193 2203 2213
GCCGAGAACA GCGUGGCCUA CUCCAACAAC UCUAUCGCUA UCCCCACCAA CUUCACCAUC
    A E N S V A Y S N N S I A I P T N E T I
                                                    S protein
            2223 2233 2243 2253 2263 2273
AGCGUGACCA CAGAGAUCCU GCCUGUGUCC AUGACCAAGA CCAGCGUGGA CUGCACCAUG
    S V T T E I L P V S M T K T S V D C T M
                                                    S protein
            2283 2293 2303 2313 2323 2333
UACAUCUGCG GCGAUUCCAC CGAGUGCUCC AACCUGCUGC UGCAGUACGG CAGCUUCUGC
    Y I C G D S T E C S N L L L Q Y G S F C
                                                    S protein
            2343 2353 2363 2373 2383 2393
ACCCAGCUGA AUAGAGCCCU GACAGGGAUC GCCGUGGAAC AGGACAAGAA CACCCAAGAG
    T Q L N R A L T G I A V E Q D K N T Q E
                                                    S protein
            2403 2413 2423 2433 2443 2453
GUGUUCGCCC AAGUGAAGCA GAUCUACAAG ACCCCUCCUA UCAAGGACUU CGGCGGCUUC
    V F A Q V K Q I Y K T P P I K D F G G F
                                                    S protein
            2463 2473 2483 2493 2503 2513
AAUUUCAGCC AGAUUCUGCC CGAUCCUAGC AAGCCCAGCA AGCGGAGCUU CAUCGAGGAC
    N F S Q I L P D P S K P S K R S F I E D
                                                    S protein
            2523 2533 2543 2553 2563 2573
CUGCUGUUCA ACAAAGUGAC ACUGGCCGAC GCCGGCUUCA UCAAGCAGUA UGGCGAUUGU
    L L F N K V T L A D A G F I K Q Y G D C
                                                    S protein
            2583 2593 2603 2613 2623 2633
CUGGGCGACA UUGCCGCCAG GGAUCUGAUU UGCGCCCAGA AGUUUAACGG ACUGACAGUG
    L G D I A A R D L I C A Q K E N G L T V
                                                    S protein
            2643 2653 2663 2673 2683 2693
CUGCCUCCUC UGCUGACCGA UGAGAUGAUC GCCCAGUACA CAUCUGCCCU GCUGGCCGGC
    L P P L L T D E M I A Q Y T S A L L A G
                                                    S protein
            2703 2713 2723 2733 2743 2753
ACAAUCACAA GCGGCUGGAC AUUUGGAGCA GGCGCCGCUC UGCAGAUCCC CUUUGCUAUG
    T I T S G W T F G A G A A L Q I P F A M
                                                    S protein
            2763 2773 2783 2793 2803 2813
CAGAUGGCCU ACCGGUUCAA CGGCAUCGGA GUGACCCAGA AUGUGCUGUA CGAGAACCAG
    Q M A Y R F N G I G V T Q N V L Y E N Q
                                                    S protein
            2823 2833 2843 2853 2863 2873
AAGCUGAUCG CCAACCAGUU CAACAGCGCC AUCGGCAAGA UCCAGGACAG CCUGAGCAGC
    K L I A N Q F N S A I G K I Q D S L S S
                                                    S protein
            2883 2893 2903 2913 2923 2933
ACAGCAAGCG CCCUGGGAAA GCUGCAGGAC GUGGUCAACC AGAAUGCCCA GGCACUGAAC
    T A S A L G K L Q D V V N Q N A Q A L N
                                                    S protein
            2943 2953 2963 2973 2983 2993
ACCCUGGUCA AGCAGCUGUC CUCCAACUUC GGCGCCAUCA GCUCUGUGCU GAACGAUAUC
    T L V K Q L S S N E G A I S S V L N D I
                                                    S protein
            3003 3013 3023 3033 3043 3053
CUGAGCAGAC UGGACCCUCC UGAGGCCGAG GUGCAGAUCG ACAGACUGAU CACAGGCAGA
    L S R L D P P E A E V Q I D R L I T G R
                                                    S protein
            3063 3073 3083 3093 3103 3113
CUGCAGAGCC UCCAGACAUA CGUGACCCAG CAGCUGAUCA GAGCCGCCGA GAUUAGAGCC
    L Q S L Q T Y V T Q Q L I R A A E I R A
                                                    S protein
            3123 3133 3143 3153 3163 3173
UCUGCCAAUC UGGCCGCCAC CAAGAUGUCU GAGUGUGUGC UGGGCCAGAG CAAGAGAGUG
    S A N L A A T K M S E C V L G Q S K R V
                                                    S protein
            3183 3193 3203 3213 3223 3233
GACUUUUGCG GCAAGGGCUA CCACCUGAUG AGCUUCCCUC AGUCUGCCCC UCACGGCGUG
    D F C G K G Y H L M S F P Q S A P H G V
                                                    S protein
            3243 3253 3263 3273 3283 3293
GUGUUUCUGC ACGUGACAUA UGUGCCCGCU CAAGAGAAGA AUUUCACCAC CGCUCCAGCC
    V F L H V T Y V P A Q E K N F T T A P A
                                                    S protein
            3303 3313 3323 3333 3343 3353
AUCUGCCACG ACGGCAAAGC CCACUUUCCU AGAGAAGGCG UGUUCGUGUC CAACGGCACC
    I C H D G K A H F P R E G V F V S N G T
                                                    S protein
            3363 3373 3383 3393 3403 3413
CAUUGGUUCG UGACACAGCG GAACUUCUAC GAGCCCCAGA UCAUCACCAC CGACAACACC
    H W F V T Q R N F Y E P Q I I T T D N T
                                                    S protein
            3423 3433 3443 3453 3463 3473
UUCGUGUCUG GCAACUGCGA CGUCGUGAUC GGCAUUGUGA ACAAUACCGU GUACGACCCU
    F V S G N C D V V I G I V N N T V Y D P
                                                    S protein
            3483 3493 3503 3513 3523 3533
CUGCAGCCCG AGCUGGACAG CUUCAAAGAG GAACUGGACA AGUACUUUAA GAACCACACA
    L Q P E L D S E K E E L D K Y F K N H T
                                                    S protein
            3543 3553 3563 3573 3583 3593
AGCCCCGACG UGGACCUGGG CGAUAUCAGC GGAAUCAAUG CCAGCGUCGU GAACAUCCAG
    S P D V D L G D I S G I N A S V V N I Q
                                                    S protein
            3603 3613 3623 3633 3643 3653
AAAGAGAUCG ACCGGCUGAA CGAGGUGGCC AAGAAUCUGA ACGAGAGCCU GAUCGACCUG
    K E I D R L N E V A K N L N E S L I D L
                                                    S protein
            3663 3673 3683 3693 3703 3713
CAAGAACUGG GGAAGUACGA GCAGUACAUC AAGUGGCCCU GGUACAUCUG GCUGGGCUUU
    Q E L G K Y E Q Y I K W P W Y I W L G F
                                                    S protein
            3723 3733 3743 3753 3763 3773
AUCGCCGGAC UGAUUGCCAU CGUGAUGGUC ACAAUCAUGC UGUGUUGCAU GACCAGCUGC
    I A G L I A I V M V T I M L C C M T S C
                                                    S protein
            3783 3793 3803 3813 3823 3833
UGUAGCUGCC UGAAGGGCUG UUGUAGCUGU GGCAGCUGCU GCAAGUUCGA CGAGGACGAU
    C S C L K G C C S C G S C C K F D E D D
                                                    S protein
            3843 3853 3863 3873 3878
UCUGAGCCCG UGCUGAAGGG CGUGAAACUG CACUACACAU GAUGA
    S E P V L K G V K L H Y T * *
                                                    S protein
            3888 3898 3908 3918 3928 3938
CUCGAGCUGG UACUGCAUGC ACGCAAUGCU AGCUGCCCCU UUCCCGUCCU GGGUACCCCG
                                                    FI element
            3948 3958 3968 3978 3988 3998
AGUCUCCCCC GACCUCGGGU CCCAGGUAUG CUCCCACCUC CACCUGCCCC ACUCACCACC
                                                    FI element
            4008 4018 4028 4038 4048 4058
UCUGCUAGUU CCAGACACCU CCCAAGCACG CAGCAAUGCA GCUCAAAACG CUUAGCCUAG
                                                    FI element
            4068 4078 4088 4098 4108 4118
CCACACCCCC ACGGGAAACA GCAGUGAUUA ACCUUUAGCA AUAAACGAAA GUUUAACUAA
                                                    FI element
            4128 4138 4148 4158 4168 4173
GCUAUACUAA CCCCAGGGUU GGUCAAUUUC GUGCCAGCCA CACCCUGGAG CUAGC
                                                    FI element
            4183 4193 4203 4213 4223 4233
AAAAAAAAAA AAAAAAAAAA AAAAAAAAAA GCAUAUGACU AAAAAAAAAA AAAAAAAAAA
                                                    Poly(A)
            4243 4253 4263 4273 4283
AAAAAAAAAA AAAAAAAAAA AAAAAAAAAA AAAAAAAAAA AAAAAAAAAA
                                                    Poly(A)
```

### Lipid Nanoparticles (LNPs)

In some embodiments, one or more nucleic acids (e.g., RNA) as described herein are formulated and/or administered in the form of LNPs. In some embodiments, a LNP of the present disclosure comprises one or more lipids known in the art and/or established herein to produce lipid particles. In some embodiments, LNPs of the present disclosure comprise one or more lipids selected from the group consisting of: cationic lipid, neutral lipid, polymer conjugated lipid, and combinations thereof. In some embodiments, LNPs of the present disclosure comprise a steroid, such as cholesterol, or derivatives thereof.

As used herein, a “neutral lipid” refers to a lipid species that exist either in an uncharged or neutral zwitterionic form at a selected pH. In some embodiments, an additional lipid comprises one of the following neutral lipid components: (1) a phospholipid, (2) cholesterol or a derivative thereof; or (3) a mixture of a phospholipid and cholesterol or a derivative thereof. In some embodiments, a phospholipid may include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

The term “cationic lipid” refers to any of a number of lipid species that carry a net positive charge at a selected pH. Such as physiological pH (e.g., pH of about 7.0). Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)—N-(3-aminopropyl)-N,N-dimethyl-2,3-bis(cis-9-tetradecenyloxy)-1-propanaminium bromide (GAP-DMORIE), (±)—N-(3-aminopropyl)-N,N-dimethyl-2,3-bis(dodecyloxy)-1-propanaminium bromide (GAP-DLRIE), (±)—N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1 S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, a cationic lipid has a chemical structure as disclosed in WO 2017/075531, some of which are set forth in Table A below:

TABLE A
Exemplary cationic lipids

| No. | Structure |
|---|---|
| I-1 | 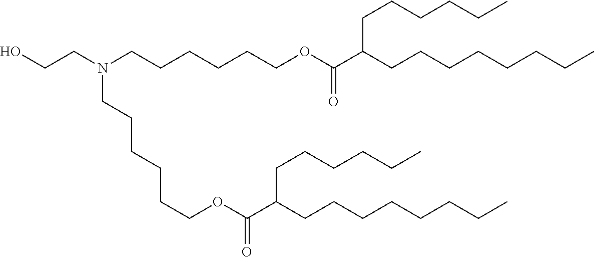 [SMILES: CCCCCCCCC(CCCCCC)C(=O)OCCCCCCN(CCO)CCCCCCOC(=O)C(CCCCCC)CCCCCCCC] |
| I-2 | 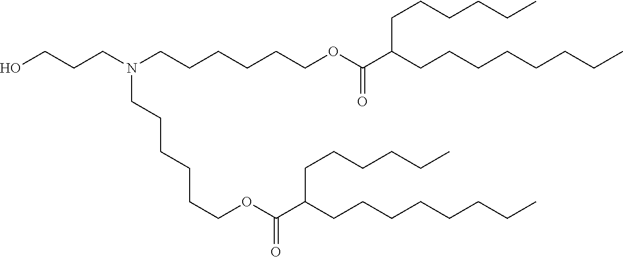 [SMILES: CCCCCCCCC(CCCCCC)C(=O)OCCCCCCN(CCCO)CCCCCCOC(=O)C(CCCCCC)CCCCCCCC] |
| I-3 | 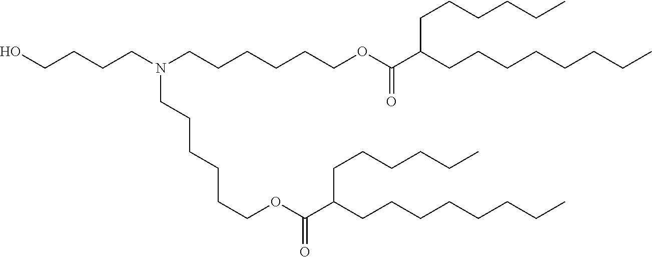 [SMILES: CCCCCCCCC(CCCCCC)C(=O)OCCCCCCN(CCCCO)CCCCCCOC(=O)C(CCCCCC)CCCCCCCC] |
| I-4 | 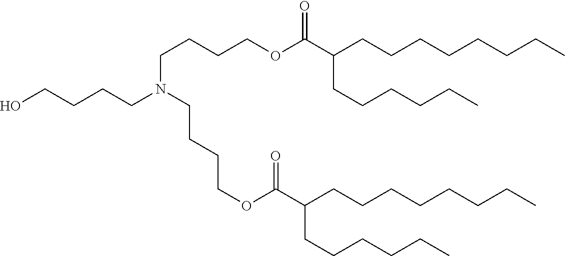 [SMILES: CCCCCCCCC(CCCCCC)C(=O)OCCCCN(CCCCO)CCCCOC(=O)C(CCCCCC)CCCCCCCC] |
| I-5 | 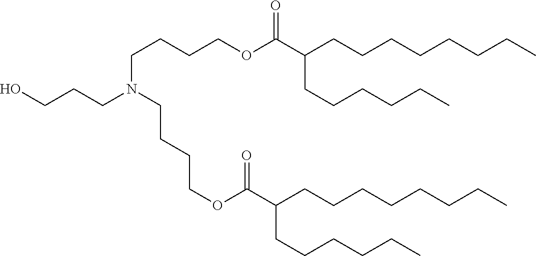 [SMILES: CCCCCCCCC(CCCCCC)C(=O)OCCCCN(CCCO)CCCCOC(=O)C(CCCCCC)CCCCCCCC] |
| I-6 | 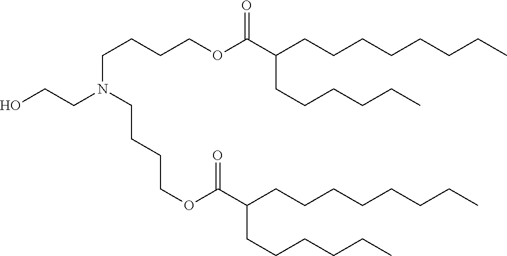 [SMILES: CCCCCCCCC(CCCCCC)C(=O)OCCCCN(CCO)CCCCOC(=O)C(CCCCCC)CCCCCCCC] |
| I-7 | 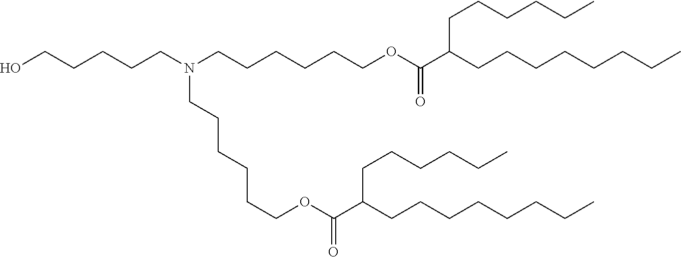 [SMILES: CCCCCCCCC(CCCCCC)C(=O)OCCCCCCN(CCCCCO)CCCCCCOC(=O)C(CCCCCC)CCCCCCCC] |
| I-8 | 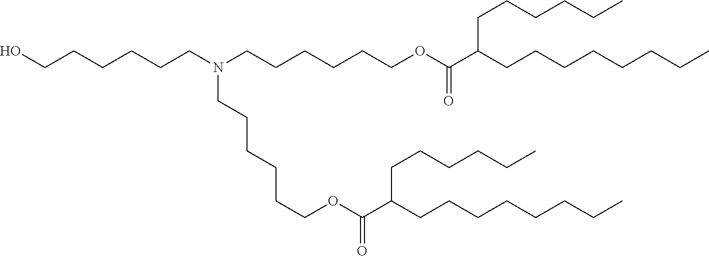 [SMILES: CCCCCCCCC(CCCCCC)C(=O)OCCCCCCN(CCCCCCO)CCCCCCOC(=O)C(CCCCCC)CCCCCCCC] |
| I-9 | 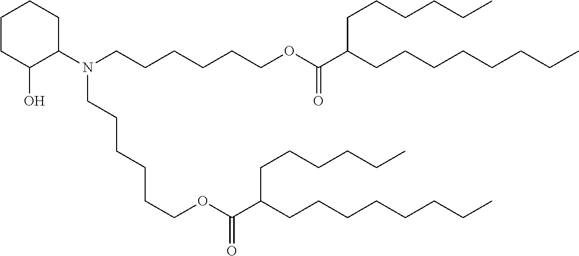 [SMILES: CCCCCCCCC(CCCCCC)C(=O)OCCCCCCN(CCCCCCOC(=O)C(CCCCCC)CCCCCCCC)C1CCCCC1O] |
| I-10 | 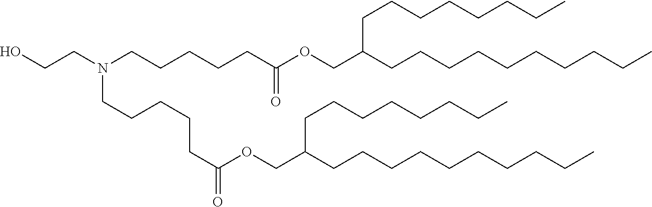 [SMILES: CCCCCCCCCCC(CCCCCCCC)COC(=O)CCCCCN(CCO)CCCCCC(=O)OCC(CCCCCCCC)CCCCCCCCCC] |
| I-11 | 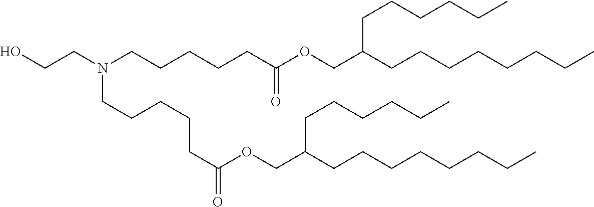 [SMILES: CCCCCCCCC(CCCCCC)COC(=O)CCCCCN(CCO)CCCCCC(=O)OCC(CCCCCC)CCCCCCCC] |
| I-12 | 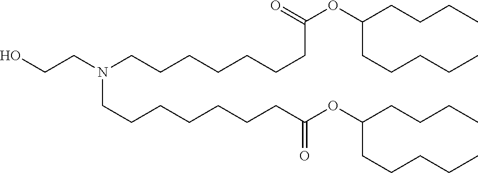 [SMILES: CCCCCC(CCCC)OC(=O)CCCCCCCN(CCO)CCCCCCCC(=O)OC(CCCC)CCCCC] |
| I-13 | 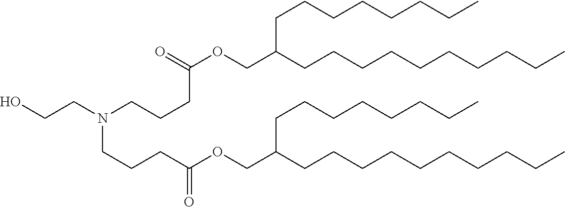 [SMILES: CCCCCCCCCCC(CCCCCCCC)COC(=O)CCCN(CCO)CCCC(=O)OCC(CCCCCCCC)CCCCCCCCCC] |
| I-14 | 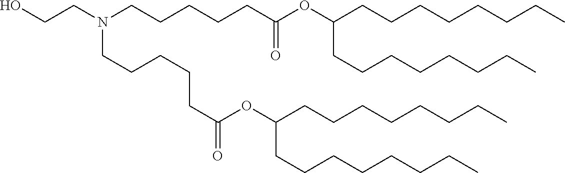 [SMILES: CCCCCCCCC(CCCCCCCC)OC(=O)CCCCCN(CCO)CCCCCC(=O)OC(CCCCCCCC)CCCCCCCC] |
| I-15 | 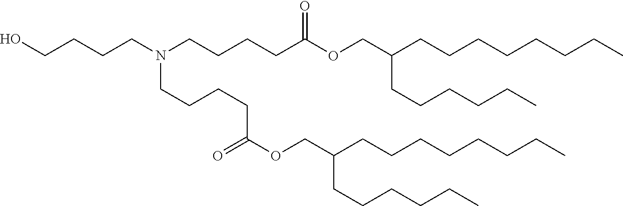 [SMILES: CCCCCCCCC(CCCCCC)COC(=O)CCCCN(CCCCO)CCCCC(=O)OCC(CCCCCC)CCCCCCCC] |
| I-16 | 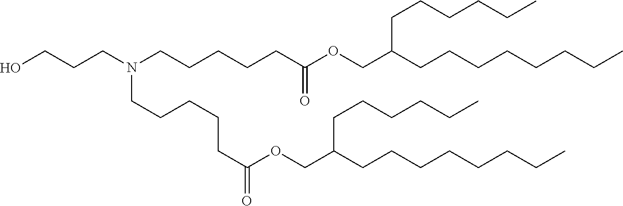 [SMILES: CCCCCCCCC(CCCCCC)COC(=O)CCCCCN(CCCO)CCCCCC(=O)OCC(CCCCCC)CCCCCCCC] |
| I-17 | 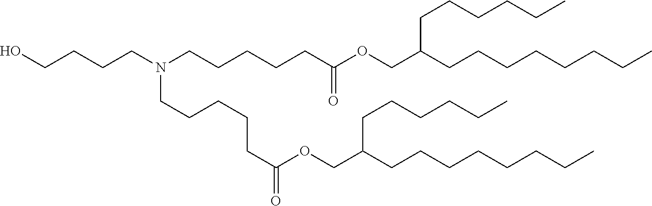 [SMILES: CCCCCCCCC(CCCCCC)COC(=O)CCCCCN(CCCCO)CCCCCC(=O)OCC(CCCCCC)CCCCCCCC] |
| I-18 | 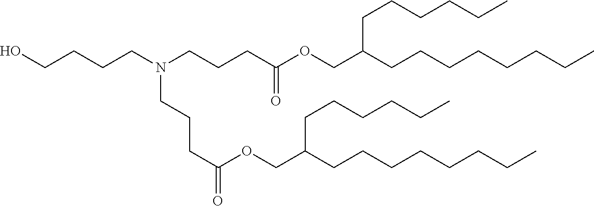 [SMILES: CCCCCCCCC(CCCCCC)COC(=O)CCCN(CCCCO)CCCC(=O)OCC(CCCCCC)CCCCCCCC] |
| I-19 | 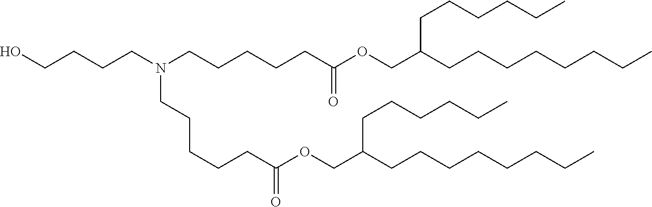 [SMILES: CCCCCCCCC(CCCCCC)COC(=O)CCCCCN(CCCCO)CCCCCC(=O)OCC(CCCCCC)CCCCCCCC] |
| I-20 | 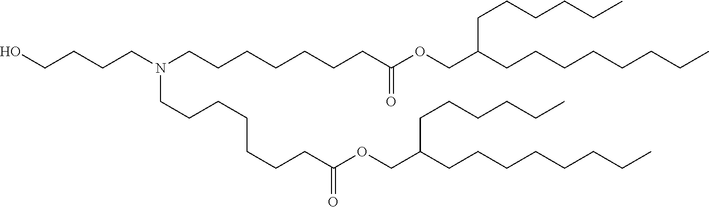 [SMILES: CCCCCCCCC(CCCCCC)COC(=O)CCCCCCCN(CCCCO)CCCCCCCC(=O)OCC(CCCCCC)CCCCCCCC] |
| I-21 | 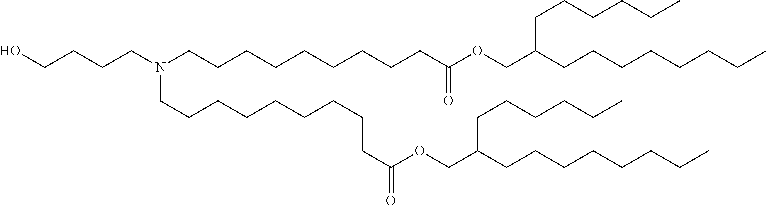 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 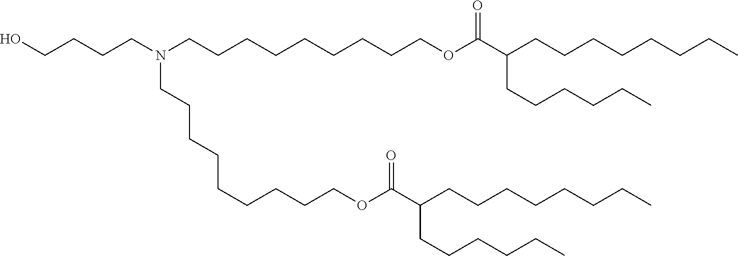 [SMILES: CCCCCCCCC(CCCCCC)C(=O)OCCCCCCCCCN(CCCCO)CCCCCCCCCOC(=O)C(CCCCCC)CCCCCCCC] |
| I-23 | 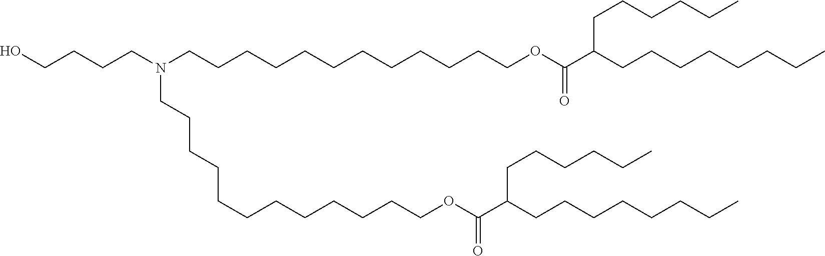 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 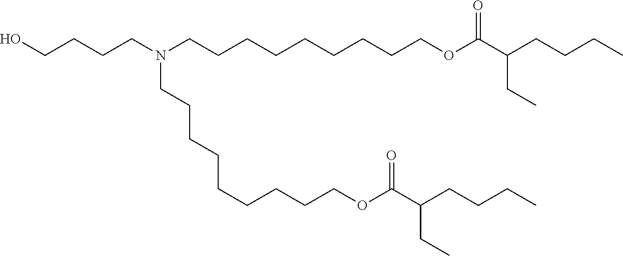 [SMILES: CCCCC(CC)C(=O)OCCCCCCCCCN(CCCCO)CCCCCCCCCOC(=O)C(CC)CCCC] |
| I-25 | 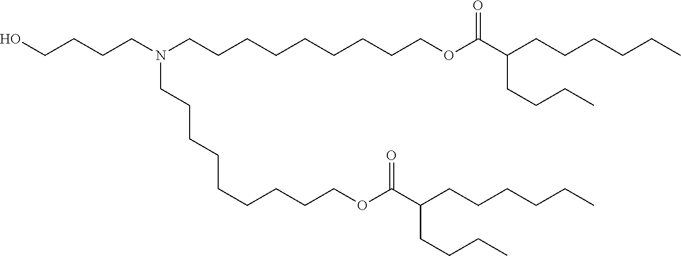 [SMILES: CCCCCCC(CCCC)C(=O)OCCCCCCCCCN(CCCCO)CCCCCCCCCOC(=O)C(CCCC)CCCCCC] |
| I-26 | 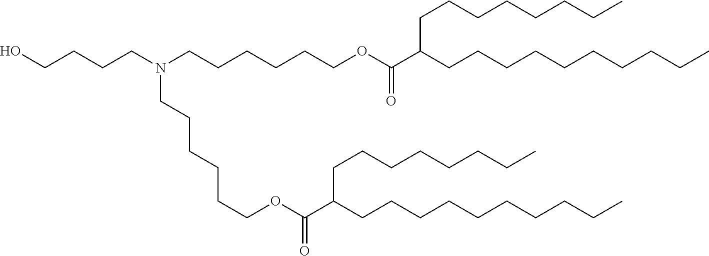 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 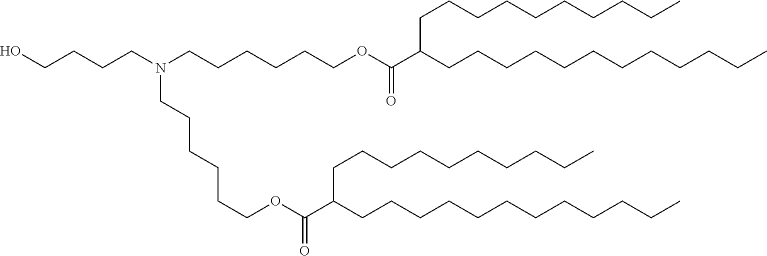 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 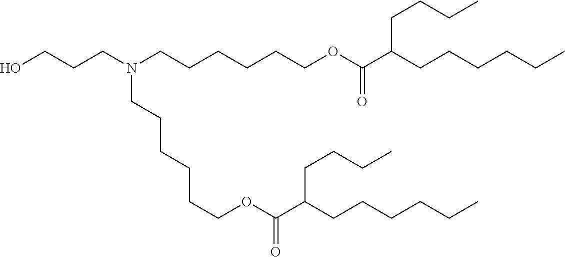 [SMILES: CCCCCCC(CCCC)C(=O)OCCCCCCN(CCCO)CCCCCCOC(=O)C(CCCC)CCCCCC] |
| I-29 | 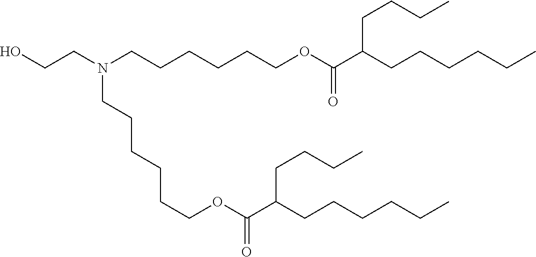 [SMILES: CCCCCCC(CCCC)C(=O)OCCCCCCN(CCO)CCCCCCOC(=O)C(CCCC)CCCCCC] |
| I-30 | 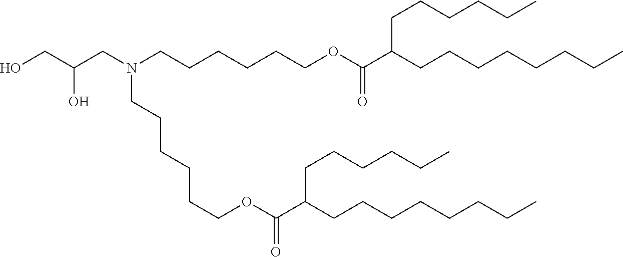 [SMILES: CCCCCCCCC(CCCCCC)C(=O)OCCCCCCN(CCCCCCOC(=O)C(CCCCCC)CCCCCCCC)CC(O)CO] |
| I-31 | 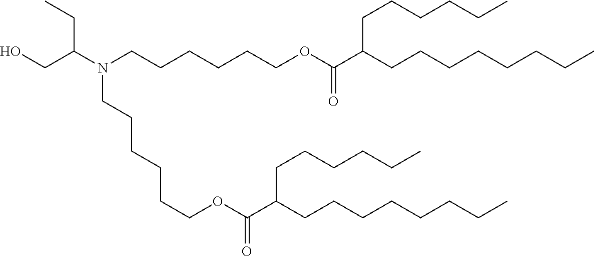 [SMILES: CCCCCCCCC(CCCCCC)C(=O)OCCCCCCN(CCCCCCOC(=O)C(CCCCCC)CCCCCCCC)C(CC)CO] |
| I-32 | 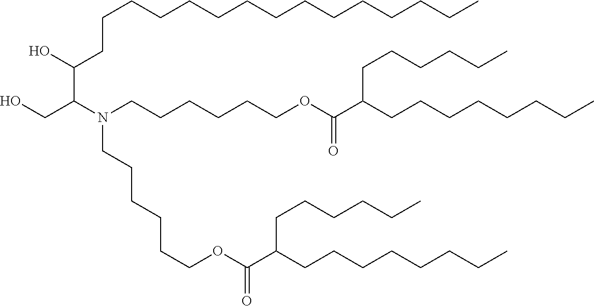 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 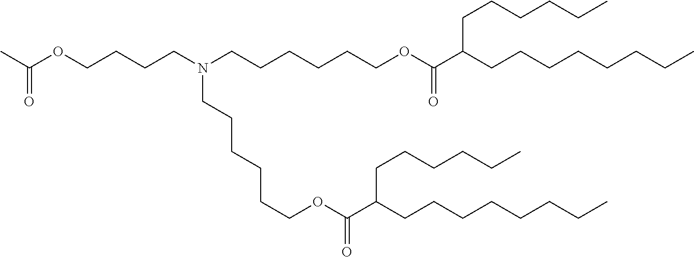 [SMILES: CCCCCCCCC(CCCCCC)C(=O)OCCCCCCN(CCCCCCOC(=O)C(CCCCCC)CCCCCCCC)CCCCOC(C)=O] |
| I-34 | 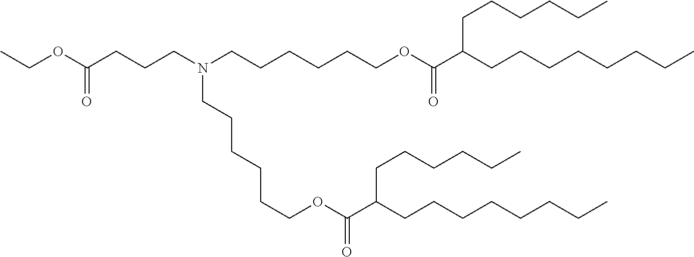 [SMILES: CCCCCCCCC(CCCCCC)C(=O)OCCCCCCN(CCCCCCOC(=O)C(CCCCCC)CCCCCCCC)CCCC(=O)OCC] |
| I-35 | 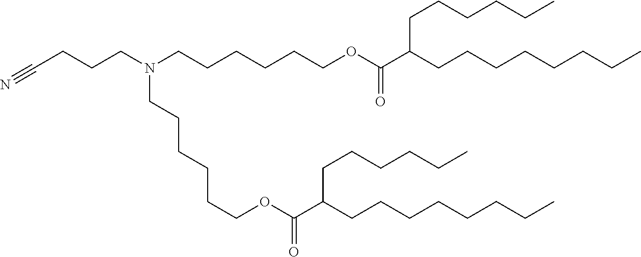 [SMILES: CCCCCCCCC(CCCCCC)C(=O)OCCCCCCN(CCCC#N)CCCCCCOC(=O)C(CCCCCC)CCCCCCCC] |
| I-36 | 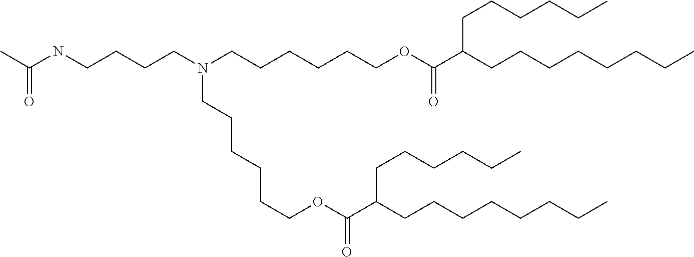 [SMILES: CCCCCCCCC(CCCCCC)C(=O)OCCCCCCN(CCCCCCOC(=O)C(CCCCCC)CCCCCCCC)CCCCNC(C)=O] |

Further examples of a cationic lipid are shown in Table B below.

TABLE B
Additional exemplary cationic lipids

| No. | Structure |
|---|---|
| A | 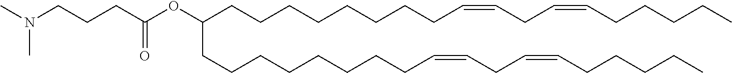 [SMILES: CCCCC/C=C\C/C=C\CCCCCCCCC(CCCCCCCC/C=C\C/C=C\CCCCC)OC(=O)CCCN(C)C] |
| B | 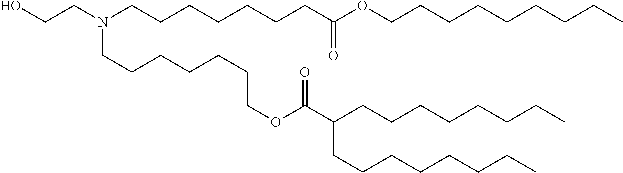 [SMILES: CCCCCCCCCOC(=O)CCCCCCCN(CCO)CCCCCCCOC(=O)C(CCCCCCCC)CCCCCCCC] |
| C | 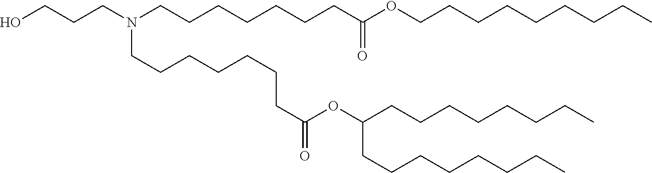 [SMILES: CCCCCCCCCOC(=O)CCCCCCCN(CCCO)CCCCCCCC(=O)OC(CCCCCCCC)CCCCCCCC] |
| D | 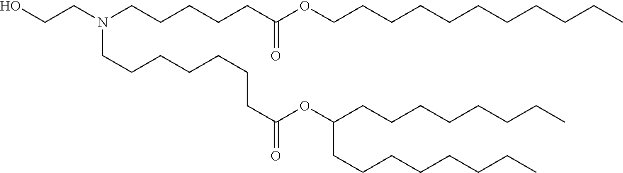 [SMILES: CCCCCCCCCCCOC(=O)CCCCCN(CCO)CCCCCCCC(=O)OC(CCCCCCCC)CCCCCCCC] |
| E | 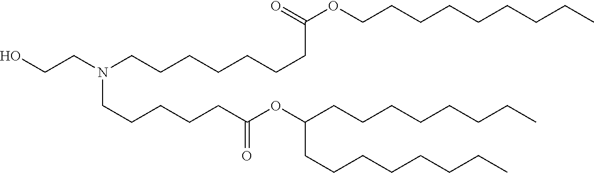 [SMILES: CCCCCCCCCOC(=O)CCCCCCCN(CCO)CCCCCC(=O)OC(CCCCCCCC)CCCCCCCC] |
| F | 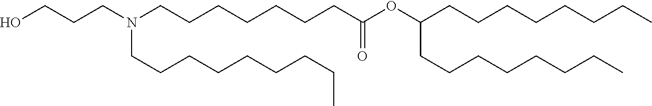 [SMILES: CCCCCCCCCN(CCCO)CCCCCCCC(=O)OC(CCCCCCCC)CCCCCCCC] |

In certain embodiments, a cationic lipid is an ionizable lipid-like material (lipidoid). An exemplary lipidoid is C12-200, which has the following structure:

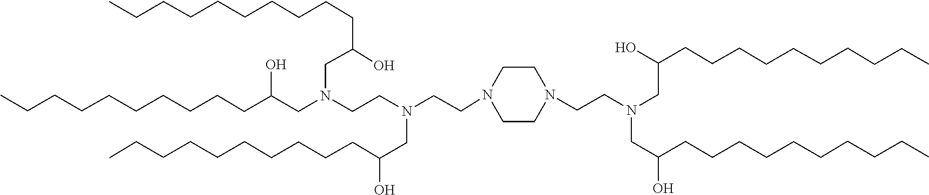 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, particles described herein include a polymer conjugated lipid such as a pegylated lipid. The term “pegylated lipid” refers to a molecule comprising both a lipid portion and a polyethylene glycol portion. Pegylated lipids are known in the art.

In some embodiments, LNPs of the present disclosure comprise ((4-hydroxybutyl)azanediyl)bis(hexane-6,1-diyl)bis(2-hexyldecanoate) (ALC-0315). In some embodiments, LNPs of the present disclosure comprise 2-[(polyethylene glycol)-2000]-N,N-ditetradecylacetamide (ALC-0159). In some embodiments, the present disclosure provides LNPs that comprise distearoylphosphatidylcholine (DSPC). In some embodiments, LNPs of the present disclosure comprise cholesterol. In some embodiments, LNPs of the present disclosure comprise lipids that include: ALC-0315, ALC-0159, DSPC, and cholesterol.

In some embodiments, a LNP of the present disclosure comprises from about 40 to about 55 mol percent, from about 40 to about 50 mol percent, from about 41 to about 49 mol percent, from about 41 to about 48 mol percent, from about 42 to about 48 mol percent, from about 43 to about 48 mol percent, from about 44 to about 48 mol percent, from about 45 to about 48 mol percent, from about 46 to about 48 mol percent, from about 47 to about 48 mol percent, or from about 47.2 to about 47.8 mol percent of ALC-0315. In some embodiments, a LNP comprises about 47.0, about 47.1, about 47.2, about 47.3, about 47.4, about 47.5, about 47.6, about 47.7, about 47.8, about 47.9, or about 48.0 mol percent of ALC-0315.

In some embodiments, a LNP of the present disclosure comprises from about 6 mg/ml to about 9 mg/ml, about 6 mg/ml to about 8 mg/ml, about 6 mg/ml to about 7 mg/ml, about 7 mg/ml to about 9 mg/ml, about 8 mg/ml to about 9 mg/ml, or about 7 mg/ml to about 8 mg/ml of ALC-0315. In some embodiments, a LNP comprises about 7 mg/ml to about 8 mg/ml of ALC-0315. In some embodiments, ALC-0315 is present in a concentration of about 7.17 mg/ml.

In some embodiment, a LNP of the present disclosure comprises from about 5 to about 15 mol percent, from about 7 to about 13 mol percent, or from about 9 to about 11 mol percent DSPC. In some embodiments, DSPC is present in a concentration of about 9.5, about 10, or about 10.5 mol percent.

In some embodiments, a LNP of the present disclosure comprises from about 1 mg/ml to about 2.5 mg/ml, about 1 mg/ml to about 2 mg/ml, or about 1 mg/ml to about 1.5 mg/ml of DSPC. In some embodiments, a LNP comprises about 1.5 mg/ml to about 2 mg/ml of DSPC. In some embodiments, ALC-0315 is present in a concentration of about 1.56 mg/ml.

In some embodiments, cholesterol is present in a concentration ranging from about 30 to about 50 mol percent, from about 35 to about 45 mol percent, or from about 38 to about 43 mol percent. In some embodiments, cholesterol is present in a concentration of about 40, about 41, about 42, about 43, about 44, about 45, or about 46 mol percent.

In some embodiments, cholesterol is present in a concentration from about 2 mg/ml to about 4 mg/ml, about 2 mg/ml to about 3.5 mg/ml, about 2 mg/ml to about 3 mg/ml, about 2 mg/ml to about 2.5 mg/ml, about 2.5 mg/ml to about 4 mg/ml, about 3 mg/ml to about 4 mg/ml, or about 3.5 mg/ml to about 4 mg/ml. In some embodiments, cholesterol is present in a concentration of about 3 mg/ml to about 3.5 mg/ml. In some embodiments, cholesterol is present in a concentration of about 3.1 mg/ml.

In some embodiments, ALC-0159 is present in a concentration ranging from about 1 to about 10 mol percent, about 2 to about 8 mol percent, about 4 to about 8 mol percent, about 4 to about 6 mol percent, about 1 to about 5 mol percent, or about 1 to about 3 mol percent.

In some embodiments, ALC-0159 is present in a concentration ranging from about 0.5 mg/ml to about 2.5 mg/ml, about 1 mg/ml to about 2.5 mg/ml, about 1.5 mg/ml to about 2.5 mg/ml, about 2 mg/ml to about 2.5 mg/ml, about 0.5 mg/ml to about 2 mg/ml, about 0.5 mg/ml to about 1.5 mg/ml, or about 0.5 mg/ml to about 1 mg/ml. In some embodiments, ALC-0159 is present in a concentration of about 0.5 mg/ml to about 1 mg/ml. In some embodiments, ALC-0159 is present in a concentration of about 0.89 mg/ml.

In some embodiments, mol percent is determined based on total mol of lipid present in LNPs described herein.

In some embodiments, the present disclosure provides LNPs comprising lipids that include ALC-0315, ALC-0159, DSPC, and cholesterol that are present in mass ratios ranging from about 8:1:1.5:3 to about 9:1:2:3.5.

In some embodiments, lipid particles of the present disclosure (e.g., LNPs) may have an average diameter of at least 30 nm, at least 40 nm, at least 50 nm, at least 60 nm, at least 70 nm, at least 80 am, at least 90 nm, at least 100 nm, at least 200 nm, at least 300 nm, at least 400 nm, at least 500 nm, or at least 1000 nm. In some embodiments, lipid particles of the present disclosure (e.g., LNPs) may have an average diameter of at most 30 nm, at most 40 nm, at most 50 nm, at most 60 nm, at most 70 nm, at most 80 nm, at most 90 nm, at most 100 nm, at most 200 nm, at most 300 nm, at most 400 nm, at most 500 nm, at most 1000 am, or at most 1200 nm. In some embodiments, lipid particles of the present disclosure (e.g., LNPs) may have an average diameter in the range of about 30 nm to about 1000 nm, about 50 nm to about 1000 nm, about 70 nm to about 1000 nm, about 30 nm to about 500 nm, about 30 nm to about 100 nm, or about 30 nm to about 80 nm.

Nucleic acids described herein can be packaged into lipids (e.g., RNA/LNPs) using a wide range of methods e.g., film hydration method, reverse phase evaporation, ethanol injection technique) that may involve obtaining a colloid from at least one cationic or cationically ionizable lipid or lipid-like material and/or at least one cationic polymer and mixing the colloid with nucleic acid to obtain lipid particles (e.g., RNA/LNPs).

In some embodiments, an RNA is packaged into a lipid particle (e.g., LNP) using an ethanol injection technique, where ethanol solution comprising lipids is rapidly injected into an aqueous solution through a needle. Accordingly, in some embodiments, nucleic acid containing lipid particles (e.g., RNA/LNPs) are made as follows: an ethanol solution comprising lipids, such as cationic lipids and additional lipids (e.g., lipid compositions as described herein), is injected into an aqueous solution comprising nucleic acid (e.g., RNA) under stirring, or agitation of the combined solution. Prepared nucleic acids in lipid particles yielded from this method can be further processed, e.g., concentrated, transferred to one or more different buffer systems, etc.

In some embodiments, an RNA as described herein is packaged into a lipid particle (e.g., LNP) by admixing said RNA with particle forming lipids (e.g., those described herein) in accordance with LNP forming methods described herein. In some embodiments, RNA containing LNPs (RNA/LNPs) are prepared in a first buffer system before being exchanged into a second buffer system for storage and/or use.

In some embodiments, a first buffer system comprises an aqueous buffer, e.g., PBS buffer, Tris buffer, HEPES buffer, His buffer, etc. In some embodiments, a first buffer system comprises a PBS buffer. In some embodiment of the present disclosure, a first buffer system comprises about 5 mg/ml to about 7 mg/ml, about 6 mg/ml to about 7 mg/ml, or about 5 mg/ml to about 6 mg/ml sodium chloride. In some embodiments, a first buffer system comprises about 6 mg/ml sodium chloride. In some embodiments, a first buffer system is substantially free of sodium chloride. One skilled in the art will understand that substantially free in this context means that no sodium chloride has been added, and that sodium and/or chloride ions may still be present due to other components in such a formulation. Accordingly, in some embodiments, PBS buffer of the present disclosure is a PBS buffer that is substantially free of sodium chloride and comprises 0.15 g/L KCl, 1.08 g/L Na₂HPO₄, and 0.15 g/L KH₂PO₄. In some embodiments, PBS of the present disclosure comprises 6 g/L NaCl, 0.15 g/L KCl, 1.08 g/L Na₂HPO₄, and 0.15 g/L KH₂PO₄.

In some embodiments, a first buffer system comprises a protectant, e.g., sucrose, trehalose, or combinations thereof. In some embodiments a protectant in a first buffer system is sucrose, and/or trehalose. In some embodiments, sucrose is at a concentration of about 10% w/v. In some embodiments, sucrose is at a concentration of about 5%. In some embodiments, trehalose is at a concentration of about 10% w/v. In some embodiments, trehalose is at a concentration of about 5%.

In some embodiments, a second buffer system of the present disclosure comprises an aqueous buffer, e.g., PBS buffer, Tris buffer, HEPES buffer, His buffer, etc. In some embodiments, a second buffer system comprises PBS. In some embodiments, PBS of the present disclosure comprises 6 g/L NaCl, 0.15 g/L KCl, 1.08 g/L Na₂HPO₄, and 0.15 g/L KH₂PO₄. In some embodiments, PBS of the present disclosure is a PBS buffer that is substantially free of sodium chloride (as defined herein), and comprises 0.15 g/L KCl, 1.08 g/L Na₂HPO₄, and 0.15 g/L KH₂PO₄. In some embodiments, a second buffer system comprises a Tris buffer. In some embodiments, a second buffer system comprises a Tris buffer at a concentration of about 10 mM. In some embodiments, a Tris buffer is substantially free of sodium chloride. In some embodiments, a Tris buffer comprises about 6 mg/ml sodium chloride. In some embodiments, a second buffer system comprises a His buffer. In some embodiments, a second buffer system comprises a His buffer at a concentration of about 10 mM. In some embodiments, a His buffer is substantially free of sodium chloride. In some embodiments, a His buffer comprises about 6 mg/ml sodium chloride. In some embodiments, a second buffer system comprises a HEPES buffer. In some embodiments, a second buffer system comprises a HEPES buffer at a concentration of about 10 mM. In some embodiments, a HEPES buffer is substantially free of sodium chloride. In some embodiments, a HEPES buffer comprises about 6 mg/ml sodium chloride.

In some embodiments, RNA-LNPs comprises about 0.4 mg/ml to about 0.6 mg/ml, about 0.4 mg/ml to about 0.5 mg/ml, or about 0.5 mg/ml to about 0.6 mg/ml mRNA. In some embodiments, RNA-LNPs comprise about 0.5 mg/ml mRNA.

### Formulations

The present disclosure provides, among other things, technologies relating to formulation of RNA therapeutics, and particular to LNP formulations comprising nucleic acid (e.g., mRNA) payloads. Such RNA/LNP formulations, include particular components (e.g., protectant and/or buffer components), and/or are prepared according to particular processes, that differ from those of a reference formulation and that modify (e.g., improve) one or more properties relative to that reference formulation. For example, in some embodiments, provided formulations show improvement(s) relative to a reference formulation that comprises the same lipids and nucleic acid, but that differs in protectant and/or buffer, and/or in certain production or processing steps.

In some embodiments, the present disclosure provides compositions that are amenable to drying and/or that are dry. In some embodiments, compositions described herein are dried by lyophilization.

In some embodiments, compositions described herein are substantially free of water, or are dried until they are substantially free of water. In some embodiments, a compositions comprises less than 0.8%, less than 0.7%, less than 0.6%, less than 0.5%, less than 0.4%, or less than 0.3% w/w water. In some embodiments, compositions as described herein maintain less than 0.8%, less than 0.7%, less than 0.6%, less than 0.5%, less than 0.4%, or less than 0.3% w/w water for a period of time, e.g., about 1, 2, 3, 4, 5, 6, weeks or more, including for 1, 2, 3, 4, 5, 6, 7, 8, 9, 10, 11, 12 months or more, and above certain low temperature thresholds, e.g., above about −80° C., −70° C., −50° C., −30° C., −20° C., 0° C., 2° C., 4° C., 8° C., 15°, 20° C., 30° C., 40° C. or higher.

In some embodiments of the present disclosure, a composition is annealed during drying (e.g., lyophilization). In some embodiments, a composition is not annealed during drying.

In some embodiments, compositions are provided that are stable to storage for at least a specified period of time at temperatures above a low temperature threshold. In some embodiments, compositions provided herein are stable to storage for a period of time at least about 1, 2, 3, 4, 5, 6, 7, 8, 9, 10, 11, 12, weeks or more, including for about 1, 2, 3, 4, 5, 6, 7, 8, 9, 10, 11, 12 months or more. In some embodiments, compositions provided herein are stable to storage for at least about 12 weeks. In some embodiments, compositions are stable to storage above a low temperature threshold that may be about −80° C., −70° C., −50° C., −30° C., −20° C., 0° C., 2° C., 4° C., 8° C., 15°, 20° C., 30° C., 40° C. or higher. In some embodiments, compositions are stable to storage at temperatures of about 0° C., 2° C., 5° C., 8° C., 25° C., 40° C. or higher. In some embodiments, compositions provided herein are stable to storage for a period of time of at least about 12 weeks at temperatures ranges of about 2° C. to about 40° C., 2° C. to about 30° C., about 2° C. to about 20° C., about 2° C. to about 10° C., about 8° C. to about 40° C., about 20° C. to about 40° C., or about 30° C. to about 40° C.

In some embodiments, a composition as described herein is considered to be stable based on maintenance of colloidal content comprising lipid nanoparticles (LNPs). In some embodiments, provided compositions described herein are considered to be stable based on maintenance of one or more of LNP characteristics (including, e.g., but not limited to its Z-average and/or polydispersity index (PDI)). In some embodiments, provided compositions described herein are considered to be stable based on maintenance of nucleic acid integrity, degree (e.g., percent) of nucleic acid encapsulation, and/or nucleic acid expressibility (e.g., level of expression of an encoded polypeptide, as may be expressed for example as percent of a relevant reference level). In some embodiments, provided compositions described herein are considered to be stable if lipid nanoparticles within such compositions exhibit less than about 20 nm change in Z-average (including, e.g., less than 19 nm, 18 nm, 17 nm, 16 nm, 15 nm, 14 nm, 13 nm, 12 nm, 11 nm, or less change in Z-average) over a certain period of time under a designated set of conditions compared to a relevant reference level. In some embodiments, provided compositions described herein are considered to be stable if lipid nanoparticles within such compositions exhibit less than about 10 nm change in Z-average (including, e.g., less than 9 nm, 8 nm, 7 nm, 6 nm, 5 nm, 4 nm, 3 nm, 2 nm, 1 nm, 0.5 nm, or less change in Z-average) over a certain period of time under a designated set of conditions compared to a relevant reference level. In some embodiments, provided compositions described herein are considered to be stable if lipid nanoparticles within such compositions exhibit less than 0.1 change in polydispersity index (PDI) (including, e.g., less than 0.09, 0.08, 0.07, 0.06, 0.05, 0.04, 0.03, or less change in PDI) over a certain period of time under a designated set of conditions compared to a relevant reference level. In some embodiments, provided compositions described herein are considered to be stable if at least 50% (including e.g., at least 60%, at least 70%, at least 80%, at least 90%, at least 95%, at least 98%, at least 99%, or more) nucleic acid encapsulation is maintained in such compositions over a certain period of time under a designated set of conditions compared to a relevant reference level. In some embodiments, provided compositions described herein are considered to be stable if at least 50% (including e.g., at least 60%, at least 70%, at least 80%, at least 90%, at least 95%, at least 98%, at least 99%, or more) of expression level of an encoded polypeptide is maintained over a certain period of time under a designated set of conditions compared to a relevant reference level.

In some embodiments, compositions (e.g., LNP compositions) as described herein are prepared in a first buffer system and then exchanged into a second buffer system as described herein.

In some embodiments, LNP compositions as described herein comprise one or more particle forming lipids. In some embodiments, particle forming lipids include: ((4-hydroxybutyl)azanediyl)bis(hexane-6,1-diyl)bis(2-hexyldecanoate) (ALC-0315), 2-[(polyethylene glycol)-2000]-N,N-ditetradecylacetamide (ALC-0159), distearoylphosphatidylcholine (DSPC), and cholesterol.

In some embodiments, LNP compositions include ALC-0315, ALC-0159, DSPC, and cholesterol, present in relative mass ratios in a range of about 8:1:1.5:3 to about 9:1:2:3.5, respectively.

In some embodiments, LNP compositions described herein include ALC-0315, ALC-0159, DSPC, and cholesterol in concentrations of 7.17 mg/ml, 0.89 mg/ml, 1.56 mg/ml, and 3.1 mg/ml, respectively.

Certain embodiments of the present disclosure utilize one or more protectants. In some embodiments, protectants are or comprise sucrose, trehalose, or combinations thereof. In some embodiments, sucrose is at a concentration of about 10% w/v in a composition or method of the present disclosure. In some embodiments, trehalose is at a concentration of about 10% w/v in a composition or method of the present disclosure. In some embodiments, sucrose is at a concentration of about 5% w/v and trehalose is at a concentration of about 5% w/v in a composition or method of the present disclosure.

In certain embodiments, a lyoprotectant is added to a composition and brought to a desired concentration (e.g., those described herein) prior to a step of freezing or a step of drying.

In some embodiments, a protectant is added to a first buffer system in which LNPs are prepared, e.g., as described herein. In some embodiments, a protectant is added to both a first buffer system and a second buffer system. In embodiments where a protectant is added to both a first buffer system and a second buffer system, a different protectant may be used for each buffer system, or the same protectant may be used. In embodiments were a protectant is added to both a first buffer system and a second buffer system, different concentrations of protectant may be used, or the same concentration may be used.

Certain embodiments of the present disclosure utilize one or more buffer systems. In some embodiments, first and second buffer systems are utilized.

In some embodiments, preparation and/or use of a provided composition may involve a step of dilution, for example by adding a buffer system, which may in some embodiments be the same as and in other embodiments may be different from a previously-used buffer system such as, for example, a buffer system included in an LNP composition that is subjected to dilution.

In some embodiments, a utilized buffer (e.g., a buffer utilized in a buffer system described herein) is substantially free of sodium chloride. One skilled in the art will understand that substantially free in this context means that no sodium chloride salt has been added, even though in some embodiments sodium and/or chloride ions may still be present due to other components in such a composition or formulation.

In some embodiments, provided compositions comprise LNPs (i.e., nucleic acid/LNPs), a protectant, and a buffer. In some embodiments, the buffer does not include sodium ions. In some embodiments the buffer does not include a salt. In some embodiments, the buffer is a HEPES buffer, a Tris buffer, or a His buffer as described herein. In some embodiments, the buffer is a phosphate buffered saline variant that is made without NaCl. In some embodiments, the buffer is a PBS variant that has a reduced level of sodium ions relative to a reference PBS that comprises NaCl, KCL, Na₂HPO₄, and KH₂PO₄; in some embodiments, such reference PBS is a “standard” PBS that comprises (or consists of) 137 mM NaCl (i.e., 8 g/L NaCl), 2.7 mM KCl (i.e., 0.2 g/L KCl), 10 mM Na₂HPO₄ (i.e., 1.44 g/L Na₂HPO₄), and 1.8 mm KH₂PO₄ (i.e., 0.24 g/L KH₂PO₄). In some embodiments, a buffer utilized in accordance with the present disclosure is a PBS variant that has a lower level of sodium ions that than found in such reference standard PBS. In some embodiments, a buffer utilized in accordance with the present disclosure is a Tris buffer at about 10 mM. In some embodiments, a buffer utilized in accordance with the present disclosure is a His buffer at about 10 mM. In some embodiments, a buffer utilized in accordance with the present disclosure is a HEPES buffer at about 10 mM. In some embodiments, a buffer utilized in accordance with the present disclosure is supplemented with 6 mg/ml sodium chloride.

In some embodiments, compositions of the present disclosure are prepared into a dosage form by dilution with a buffer.

### Uses

As described herein, technologies provided by the present disclosure relate to and/or are useful for preparation and/or administration of one or more nucleic acid/LNP (e.g., RNA/LNP) compositions.

In some embodiments, technologies described herein provide LNP compositions (e.g., LNP/RNA compositions) that are stable to storage for a period of time at least about 1, 2, 3, 4, 5, 6, 7, 8, 9, 10, 11, 12, weeks or more, including for about 1, 2, 3, 4, 5, 6, 7, 8, 9, 10, 11, 12 months or more. In some embodiments, technologies of the present disclosure provide LNP compositions that are stable to storage for at least about 12 weeks. In some embodiments, provided compositions are stable to storage above a low temperature threshold that may be about −80° C., −70° C., −50° C., −30° C., −20° C., 0° C., 2° C., 4° C., 8° C., 15°, 20° C., 30° C., 40° C. or higher. In some embodiments, provided compositions are stable to storage at temperatures of about 0° C., 2° C. 5° C., 8° C., 25° C., 40° C. or higher. In some embodiments, LNP compositions provided herein are stable to storage for a period of time of at least about 12 weeks at temperatures ranges of about 2° C. to about 40° C., 2° C. to about 30° C., about 2° C. to about 20° C., about 2° C. to about 10° C., about 8° C. to about 40° C., about 20° C. to about 40° C., or about 30° C. to about 40° C.

Provided compositions described herein are considered to be stable based on maintenance of colloidal content comprising lipid nanoparticles (LNPs). In some embodiments, provided compositions described herein are considered to be stable based on maintenance of one or more of LNP characteristics (including, e.g., but not limited to its Z-average and/or polydispersity index (PDI)). In some embodiments, provided compositions described herein are considered to be stable based on maintenance of nucleic acid integrity, degree (e.g., percent) of nucleic acid encapsulation, and/or nucleic acid expressibility (e.g., level of expression of an encoded polypeptide, as may be expressed for example as percent of a relevant reference level). In some embodiments, provided compositions described herein are considered to be stable if lipid nanoparticles within such compositions exhibit less than about 20 nm change in Z-average (including, e.g., less than 19 nm, 18 nm, 17 nm, 16 nm, 15 nm, 14 nm, 13 nm, 12 nm, 11 nm, or less change in Z-average) over a certain period of time under a designated set of conditions compared to a relevant reference level. In some embodiments, provided compositions described herein are considered to be stable if lipid nanoparticles within such compositions exhibit less than about 10 nm change in Z-average (including, e.g., less than 9 nm, 8 nm, 7 nm, 6 nm, 5 nm, 4 nm, 3 nm, 2 nm, 1 nm, 0.5 nm, or less change in Z-average) over a certain period of time under a designated set of conditions compared to a relevant reference level. In some embodiments, provided compositions described herein are considered to be stable if lipid nanoparticles within such compositions exhibit less than 0.1 change in polydispersity index (PDI) (including, e.g., less than 0.09, 0.08, 0.07, 0.06, 0.05, 0.04, 0.03, or less change in PDI) over a certain period of time under a designated set of conditions compared to a relevant reference level. In some embodiments, provided compositions described herein are considered to be stable if at least 50% (including e.g., at least 60%, at least 70%, at least 80%, at least 90%, at least 95%, at least 98%, at least 99%, or more) nucleic acid encapsulation is maintained in such compositions over a certain period of time under a designated set of conditions compared to a relevant reference level. In some embodiments, provided compositions described herein are considered to be stable if at least 50% (including e.g., at least 60%, at least 70%, at least 80%, at least 90%, at least 95%, at least 98%, at least 99%, or more) of expression level of an encoded polypeptide is maintained over a certain period of time under a designated set of conditions compared to a relevant reference level.

In some embodiments, technologies provided herein utilize an antigen that may be or comprise a viral antigen, e.g. an antigen associated with a virus selected from the group consisting of: adenovirus, cytomegalovirus, herpes virus, human papillomavirus, measles virus, rubella virus, coronavirus, respiratory syncytial virus, influenza virus, and mumps virus. In some embodiments, an antigen may be or comprise a viral antigen associated with a virus selected from a Class I, Class 11, Class III, Class IV, Class V, Class VI, or Class VII virus, based on the Baltimore classification system. In some embodiments, technologies described herein provide immunity in a subject from a virus selected from viral family Adenoviridae, Papovaviridae, Parvoviridae, Herpesviridae, Poxviridae, Anelloviridae, Pleolipoviridae, Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae, Arteriviridae, or Hepeviridae. In some embodiments, technologies described herein provide immunity in a subject to a viral infection. In some embodiments, technologies described herein provide immunity in a subject to coronavirus, coronavirus infection, or to a disease or disorder associated with coronavirus. The present disclosure thus provides compositions and methods for treating or preventing an infection, disease, or disorder associated with coronavirus.

In some embodiments, technologies described herein provide LNP compositions that are administered to a subject having an infection, disease, or disorder associated with coronavirus. In some embodiments, technologies described herein provide LNP compositions that are administered to a subject at risk for developing the infection, disease, or disorder associated with coronavirus. For example, technologies described herein provide LNP compositions that may be administered to a subject who is at risk for being in contact with coronavirus. In some embodiments, technologies described herein provide LNP compositions that are administered to a subject who lives in, traveled to, or is expected to travel to a geographic region in which coronavirus is prevalent. In some embodiments, technologies described herein provide LNP compositions that are administered to a subject who is in contact with or expected to be in contact with another person who lives in, traveled to, or is expected to travel to a geographic region in which coronavirus is prevalent. In some embodiments, technologies described herein provide LNP compositions that are administered to a subject who has knowingly been exposed to coronavirus through their occupation, or other contact. In some embodiments, a coronavirus is SARS-CoV-2.

In some embodiments, technologies described herein provide compositions that may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present disclosure, prophylactic administration occurs prior to the manifestation of overt clinical symptoms of disease, such that a disease or disorder is prevented (e.g., reduce burden of mortality or morbidity of disease) or alternatively delayed in its progression.

### Administration

Provided herein are compositions (e.g., pharmaceutical compositions) and methods for delivering a payload (e.g., mRNA) to a cell in a subject in need of such a payload. In some embodiments, provided compositions are administered for prophylactic purposes against a viral infection and/or therapeutic purposes to treat a viral infection. In some embodiments, technologies of the present disclosure provide for compositions that can be used as therapeutic or prophylactic agents for treatment of coronavirus, e.g., SARS-CoV-2.

Pharmaceutical compositions of the present disclosure may be administered to prophylactic purposes, e.g., in a subject that has not been diagnosed, and/or has not displayed one or more particular symptoms or characteristics of a particular disease, disorder or condition. In some embodiments, pharmaceutical compositions provided herein are administered in amounts to a cell or tissue of a subject in amounts effective for immune prophylaxis. Pharmaceutical compositions provided herein may be administered with other therapeutic or prophylactic compounds.

In some embodiments, pharmaceutical compositions are administered therapeutically, e.g., in a subject that has been diagnosed, and/or has displayed one or more particular symptoms or characterisitics of a particular disease, disorder, or condition. In some embodiments, pharmaceutical compositions provided herein are administered in amounts to a cell or tissue of a subject in therapeutically effective amounts. Such pharmaceutical compositions provided herein may be administered with other therapeutic or prophylactic compounds.

The exact amount of a provided pharmaceutical composition (e.g., RNA/LNP composition) required for prophylactic and/or therapeutic purposes will vary from subject to subject, depending on the species, age, and general condition of the subject, severity of the disease, mode of administration, and mode of activity, among other considerations. It will be understood, however, that usage of provided compositions may be decided by the attending physician within the scope of sound medical judgment. Accordingly, a specific therapeutically and/or prophylactically effective dose for a particular patient will depend upon a variety of factors including the disorder being treated and the severity of the disorder, the activity or potency of the specific composition employed, the age, body weight, general health, sex, and diet of the patient, time of administration, route of administration, and rate of excretion of the specific compound employed, duration of the treatment; drugs used in combination or coincidental with the specific compound employed, and like factors well known in the medical arts.

In some embodiments, provided pharmaceutical compositions are administered to a subject who has received, is receiving, or will receive other therapy. In some embodiments, other therapies administered with, e.g., concomitantly, or in an alternating regimen, address one or more symptoms or features of a disease, disorder, or condition treated by provided therapy. Alternatively, or additionally, in some embodiments, an other therapy addresses one or more symptoms or features of a different disease. To give but one example, in various embodiments, it may be desirable to administer a plurality of prophylactic therapies (e.g., prophylactic vaccines) substantially contemporaneously.

Pharmaceutical compositions described herein may comprise one or more adjuvants or may be administered in combination with (i.e., may be administered to subjects who have received, will receive, or are receiving) one or more adjuvants. An adjuvant utilized in the present disclosure may relate to any compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. Cytokines utilized in accordance with the present disclosure may be IL1, IL2, IL3, IL4, IL5, IL6, IL7, IL8, IL9, IL10, IL12, IFNα, IFNγ, GM-CSF, LT-a, or combinations thereof. Further known adjuvants that may be used in accordance with the present disclosure are aluminium hydroxide, Freund's adjuvant or oil such as Montanide® ISA51. Other suitable adjuvants for use in the present disclosure include lipopeptides, such as Pam3Cys.

Pharmaceutical compositions described herein may be provided as a frozen concentrate for solution for injection, e.g., at a concentration of about 0.50 mg/mL. In some embodiments, for preparation of solution for injection, a drug product is thawed and diluted, and/or rehydrated and diluted, with isotonic sodium chloride solution (e.g., 0.9% NaCl, saline), e.g., by a one-step dilution process. The concentration of the final solution for injection varies depending on the respective dose level to be administered.

In some embodiments, an amount of RNA described herein from 0.1 μg to 300 μg, 0.5 μg to 200 μg, or 1 μg to 100 μg, such as about 1 μg, about 3 μg, about 10 μg, about 30 μg, about 50 μg, or about 100 μg may be administered per dose. In some embodiments, the disclosure compositions described herein are administered in single dose. In some embodiments, compositions described herein are administered in a priming dose followed by one or more booster doses. In some embodiments, a booster dose or a first booster dose may be administered 7 to 28 days or 14 to 24 days following administration of a priming dose.

In some embodiments, an amount of RNA described herein of 60 μg or lower, 50 μg or lower, 40 μg or lower, 30 μg or lower, 20 μg or lower, 10 μg or lower, 5 μg or lower, 2.5 μg or lower, or 1 μg or lower may be administered per dose.

In some embodiments, an amount of RNA described herein of at least 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg may be administered per dose.

In some embodiments, an amount of RNA described herein of 0.25 μg to 60 μg, 0.5 μg to 55 μg, 1 μg to 50 μg, 5 μg to 40 μg, or 10 μg to 30 μg may be administered per dose.

In some embodiments, an amount of RNA described herein of about 30 μg is administered per dose. In some embodiments, at least two of such doses are administered. For example, a second dose may be administered about 21 days following administration of a first dose.

In some embodiments, RNA administered as described above is nucleoside modified messenger RNA (modRNA) described herein as BNT162b2 (RBP020.1 or RBP020.2). In some embodiments, RNA administered as described above is nucleoside modified messenger RNA (modRNA) described herein as RBP020.2.

In some embodiments, administration of an immunogenic composition or vaccine of the present disclosure may be performed by single administration or boosted by multiple administrations.

### EXEMPLIFICATION

The following Examples are provided for illustration and are not in any way to limit the scope of the disclosure. One of skill in the art will appreciate that certain design and selection criteria as described herein may be changed according to common practices in the field.

### Example 1: Exemplary Compositions and Characterization

The present example describes development and/or characterization of certain RNA/LNP compositions in accordance with the present disclosure.

The RNA payload utilized in the present Example was a modified RNA payload in that it included 4283 nucleotide residues. The RNA payload utilized in the present Example encoded a viral antigen, in particular the SARS-CoV-2 S protein. Specifically, the RNA payload utilized in the present Example was the BNT162b2 construct as represented by RBP020.2 (v9) described herein.

The present example assessed certain protectants (specifically disacharide protectants sucrose and trehalose) and particular buffers (e.g., non-phosphate buffers such as Tris and histidine buffers, and/or buffers that do not include NaCl).

Without wishing to be bound by any particular theory, it was considered that isotonicity might be desirable; certain assessed compositions included protectant at a concentration of 10% w/v since it yields a nearly isotonic solution.

Buffer concentration was selected to be sufficient for maintaining pH of the compositions.

Assessed compositions did not include mannitol.

In this example, compositions were not frozen (e.g., were maintained at a temperature within a range of about 2° C. to about 8° C.) prior to drying. In this example, drying was performed by freeze-drying (specifically, lyophilization).

Electrical conductivity of compositions was measured, and low temperature DSC experiments were performed prior to initiation of freeze-drying. LNP size and polydispersity were determined using dynamic light scattering after the 2° C.-8° C. temperature hold and prior to lyophilization.

Table 1 below presents certain assessed compositions; as can be seen, (i) protectant type and concentration was varied; an alternate fabrication process was assessed (specifically for a sucrose-containing formulation, RNA stock was diluted into a sucrose-citrate buffer, rather than a citrate-only buffer) during fabrication; (ii) buffers lacking NaCl were assessed; and (iii) non-phosphate buffers (e.g., Tris, His, HEPES) were assessed.

TABLE 1
Compositions of formulations for lyophilization assessment

| Formulation | | | | Volume, |
|---|---|---|---|---|
| # | Buffer | Protectant | Notes | mL |
| 1 | 10 mM Tris, pH 7.4 | 10% w/v Sucrose | alternate fabrication | 15 + 15 = 30* |
| | | | process | |
| 2 | 10 mM Tris, pH 7.4 | 10% w/v Trehalose | — | 15 + 15 = 30* |
| 3 | 10 mM Tris, pH 7.4 | 5% w/v Sucrose | — | 15 + 15 = 30* |
| | | 5% w/v Trehalose | | |
| 4 | 10 mM His, pH 7.4 | 10% w/v Sucrose | alternate fabrication | 15 + 15 = 30* |
| | | | process | |

*Two aliquots of 15 mL each should be prepared and stored separately for lyophilization cycles with and without annealing during freezing.

The utilized lyophilization process involved cooling and warming ramps during the freezing step that were performed at 0.5° C./min. The formulations were frozen to a temperature below Tg′ of the relevant formulation. Without wishing to be bound by any particular theory, an annealing temperature of −10° C. was selected to maximize Ostwald ripening during the isothermal hold (and thereby increase the size of ice crystals) and decrease cake resistance while keeping product below melting point of formulations. The ramp rate to secondary drying was 0.2° C./min.

We claim:

1. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) Tris buffer, wherein the Tris buffer is substantially free of sodium chloride and is at a concentration of about 10 mM in the formulation.

2. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) Tris buffer, wherein the Tris buffer is substantially free of sodium chloride and is at a concentration of about 10 mM in the formulation.

3. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation before drying;
c) Tris buffer, wherein the Tris buffer is substantially free of sodium chloride and is at a concentration of about 10 mM in the formulation before drying.

4. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) Tris buffer, wherein the Tris buffer is substantially free of sodium chloride and is at a concentration of about 10 mM in the formulation; and
ii) sucrose at a concentration of about 10% w/v in the formulation.

5. The method of claim 4, further comprising a step of:
c) freezing the formulation.

6. The method of claim 4, further comprising a step of:
c) drying the formulation.

7. A method of preparing a dosage form, the method comprising a step of:
a) diluting a formulation of claim 1;
b) thawing and diluting a formulation of claim 2; and/or
c) resuspending and diluting a formulation of claim 3.

8. The method of claim 7, further comprising administering the dosage form to a subject in need of thereof.

9. The method of claim 8, wherein the subject is in need of an expression product of the mRNA.

10. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 10% w/v;
c) Tris buffer, wherein the Tris buffer is substantially free of sodium chloride and is at a concentration of about 10 mM in the formulation;
wherein the formulation is diluted into the dosage form prior to administration.

11. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) Tris buffer, wherein the Tris buffer is substantially free of sodium chloride and is at a concentration of about 10 mM in the formulation.

12. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) Tris buffer, wherein the Tris buffer is substantially free of sodium chloride and is at a concentration of about 10 mM in the formulation.

13. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation before drying;
c) Tris buffer, wherein the Tris buffer is substantially free of sodium chloride and is at a concentration of about 10 mM in the formulation before drying.

14. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) Tris buffer, wherein the Tris buffer is substantially free of sodium chloride and is at a concentration of about 10 mM in the formulation; and
ii) trehalose at a concentration of about 10% w/v in the formulation.

15. The method of claim 14, further comprising a step of:
c) freezing the formulation.

16. The method of claim 14, further comprising a step of:
c) drying the formulation.

17. A method of preparing a dosage form, the method comprising a step of:
a) diluting a formulation of claim 11;
b) thawing and diluting a formulation of claim 12; and/or
c) resuspending and diluting a formulation of claim 13.

18. The method of claim 17, further comprising administering the dosage form to a subject in need of thereof.

19. The method of claim 18, wherein the subject is in need of an expression product of the mRNA.

20. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) trehalose at a concentration of about 10% w/v;
c) Tris buffer, wherein the Tris buffer is substantially free of sodium chloride and is at a concentration of about 10 mM in the formulation;
wherein the formulation is diluted into the dosage form prior to administration.

21. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) Tris buffer, wherein the Tris buffer is substantially free of sodium chloride and is at a concentration of about 10 mM in the formulation.

22. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) Tris buffer, wherein the Tris buffer is substantially free of sodium chloride and is at a concentration of about 10 mM in the formulation.

23. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation before drying;
c) trehalose at a concentration of about 5% w/v in the formulation before drying;
d) Tris buffer, wherein the Tris buffer is substantially free of sodium chloride and is at a concentration of about 10 mM in the formulation before drying.

24. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) Tris buffer, wherein the Tris buffer is substantially free of sodium chloride and is at a concentration of about 10 mM in the formulation;
ii) sucrose at a concentration of about 5% w/v in the formulation; and
iii) trehalose at a concentration of about 5% w/v in the formulation.

25. The method of claim 24, further comprising a step of:
c) freezing the formulation.

26. The method of claim 24, further comprising a step of:
c) drying the formulation.

27. A method of preparing a dosage form, the method comprising a step of:
a) diluting a formulation of claim 21;
b) thawing and diluting a formulation of claim 22; and/or
c) resuspending and diluting a formulation of claim 23.

28. The method of claim 27, further comprising administering the dosage form to a subject in need of thereof.

29. The method of claim 28, wherein the subject is in need of an expression product of the mRNA.

30. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 5% w/v in the formulation;
c) trehalose at a concentration of about 5% w/v in the formulation;
d) Tris buffer, wherein the Tris buffer is substantially free of sodium chloride and is at a concentration of about 10 mM in the formulation;
wherein the formulation is diluted into the dosage form prior to administration.

31. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) Tris buffer, wherein the Tris buffer comprises about 6 mg/ml sodium chloride and is at a concentration of about 10 mM in the formulation.

32. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) Tris buffer, wherein the Tris buffer comprises about 6 mg/ml sodium chloride and is at a concentration of about 10 mM in the formulation.

33. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation before drying;
c) Tris buffer, wherein the Tris buffer comprises about 6 mg/ml sodium chloride and is at a concentration of about 10 mM in the formulation before drying.

34. A method of preparing a formulation comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) Tris buffer, wherein the Tris buffer comprises about 6 mg/ml sodium chloride and is at a concentration of about 10 mM in the formulation; and
ii) sucrose at a concentration of about 10% w/v in the formulation.

35. The method of claim 34, further comprising a step of:
c) freezing the formulation.

36. The method of claim 34, further comprising a step of:
c) drying the formulation.

37. A method of preparing a dosage form, the method comprising a step of:
a) diluting a formulation of claim 31;
b) thawing and diluting a formulation of claim 32; and/or
c) resuspending and diluting a formulation of claim 33.

38. The method of claim 37, further comprising administering the dosage form to a subject in need of thereof.

39. The method of claim 38, wherein the subject is in need of an expression product of the mRNA.

40. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/mL;
b) sucrose at a concentration of about 10% w/v in the formulation;
c) Tris buffer, wherein the Tris buffer comprises about 6 mg/ml sodium chloride and is at a concentration of about 10 mM in the formulation;
wherein the formulation is diluted into the dosage form prior to administration.

41. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) Tris buffer, wherein the Tris buffer comprises about 6 mg/ml sodium chloride and is at a concentration of about 10 mM in the formulation.

42. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) Tris buffer, wherein the Tris buffer comprises about 6 mg/ml sodium chloride and is at a concentration of about 10 mM in the formulation.

43. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation before drying;
c) Tris buffer, wherein the Tris buffer comprises about 6 mg/ml sodium chloride and is at a concentration of about 10 mM in the formulation before drying.

44. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) Tris buffer, wherein the Tris buffer comprises about 6 mg/ml sodium chloride and is at a concentration of about 10 mM in the formulation; and
ii) trehalose at a concentration of about 10% w/v in the formulation.

45. The method of claim 44, further comprising a step of:
c) freezing the formulation.

46. The method of claim 44, further comprising a step of:
c) drying the formulation.

47. A method of preparing a dosage form, the method comprising a step of:
a) diluting a formulation of claim 41;
b) thawing and diluting a formulation of claim 42; and/or
c) resuspending and diluting a formulation of claim 43.

48. The method of claim 47, further comprising administering the dosage form to a subject in need of thereof.

49. The method of claim 48, wherein the subject is in need of an expression product of the mRNA.

50. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) trehalose at a concentration of about 10% w/v in the formulation;
c) Tris buffer, wherein the Tris buffer comprises about 6 mg/ml sodium chloride and is at a concentration of about 10 mM in the formulation;
wherein the formulation is diluted into the dosage form prior to administration.

51. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) Tris buffer, wherein the Tris buffer comprises about 6 mg/ml sodium chloride and is at a concentration of about 10 mM in the formulation.

52. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) Tris buffer, wherein the Tris buffer comprises about 6 mg/ml sodium chloride and is at a concentration of about 10 mM in the formulation.

53. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation before drying:
c) trehalose at a concentration of about 5% w/v in the formulation before drying;
d) Tris buffer, wherein the Tris buffer comprises about 6 mg/ml sodium chloride and is at a concentration of about 10 mM in the formulation before drying.

54. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) Tris buffer, wherein the Tris buffer comprises about 6 mg/ml sodium chloride and is at a concentration of about 10 mM in the formulation;
ii) sucrose at a concentration of about 5% w/v in the formulation; and
iii) trehalose at a concentration of about 5% w/v in the formulation.

55. The method of claim 54, further comprising a step of:
c) freezing the formulation.

56. The method of claim 54, further comprising a step of:
c) drying the formulation.

57. A method of preparing a dosage form, the method comprising a step of:
a) diluting a formulation of claim 51;
b) thawing and diluting a formulation of claim 52; and/or
c) resuspending and diluting a formulation of claim 53.

58. The method of claim 57, further comprising administering the dosage form to a subject in need of thereof.

59. The method of claim 58, wherein the subject is in need of an expression product of the mRNA.

60. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 5% w/v in the formulation;
c) trehalose at a concentration of about 5% w/v in the formulation;
d) Tris buffer, wherein the Tris buffer comprises about 6 mg/ml sodium chloride and is at a concentration of about 10 mM in the formulation;
wherein the formulation is diluted into the dosage form prior to administration.

61. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) His buffer, wherein the His buffer is substantially free of sodium chloride and is at a concentration of about 10 mM in the formulation.

62. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) His buffer, wherein the His buffer is substantially free of sodium chloride and is at a concentration of about 10 mM in the formulation.

63. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation before drying;
c) His buffer, wherein the His buffer is substantially free of sodium chloride and is at a concentration of about 10 mM in the formulation before drying.

64. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) His buffer, wherein the His buffer is substantially free of sodium chloride and is at a concentration of about 10 mM in the formulation; and
ii) sucrose at a concentration of about 10% w/v in the formulation.

65. The method of claim 64, further comprising a step of:
c) freezing the formulation.

66. The method of claim 64, further comprising a step of:
c) drying the formulation.

67. A method of preparing a dosage form, the method comprising a step of:
a) diluting a formulation of claim 61;
b) thawing and diluting a formulation of claim 62; and/or
c) resuspending and diluting a formulation of claim 63.

68. The method of claim 67, further comprising administering the dosage form to a subject in need of thereof.

69. The method of claim 68, wherein the subject is in need of an expression product of the mRNA.

70. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 10% w/v in the formulation;
d) His buffer, wherein the His buffer is substantially free of sodium chloride and is at a concentration of about 10 mM in the formulation;
wherein the formulation is diluted into the dosage form prior to administration.

71. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) His buffer, wherein the His buffer is substantially free of sodium chloride and is at a concentration of about 10 mM in the formulation.

72. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) His buffer, wherein the His buffer is substantially free of sodium chloride and is at a concentration of about 10 mM in the formulation.

73. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation before drying;
c) His buffer, wherein the His buffer is substantially free of sodium chloride and is at a concentration of about 10 mM in the formulation before drying.

74. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) His buffer, wherein the His buffer is substantially free of sodium chloride and is at a concentration of about 10 mM in the formulation; and
ii) trehalose at a concentration of about 10% w/v in the formulation.

75. The method of claim 74, further comprising a step of:
c) freezing the formulation.

76. The method of claim 74, further comprising a step of:
c) drying the formulation.

77. A method of preparing a dosage form, the method comprising a step of:
a) diluting a formulation of claim 71;
b) thawing and diluting a formulation of claim 72; and/or
c) resuspending and diluting a formulation of claim 73.

78. The method of claim 77, further comprising administering the dosage form to a subject in need of thereof.

79. The method of claim 78, wherein the subject is in need of an expression product of the mRNA.

80. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) trehalose at a concentration of about 10% w/v in the formulation;
d) His buffer, wherein the His buffer is substantially free of sodium chloride and is at a concentration of about 10 mM in the formulation;
wherein the formulation is diluted into the dosage form prior to administration.

81. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) His buffer, wherein the His buffer is substantially free of sodium chloride and is at a concentration of about 10 mM in the formulation.

82. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) His buffer, wherein the His buffer is substantially free of sodium chloride and is at a concentration of about 10 mM in the formulation.

83. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation before drying;
c) trehalose at a concentration of about 5% w/v in the formulation before drying;
d) His buffer, wherein the His buffer is substantially free of sodium chloride and is at a concentration of about 10 mM in the formulation before drying.

84. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) His buffer, wherein the His buffer is substantially free of sodium chloride and is at a concentration of about 10 mM in the formulation;
ii) sucrose at a concentration of about 5% w/v in the formulation; and
iii) trehalose at a concentration of about 5% w/v in the formulation.

85. The method of claim 84, further comprising a step of:
c) freezing the formulation.

86. The method of claim 84, further comprising a step of:
c) drying the formulation.

87. A method of preparing a dosage form, the method comprising a step of:
a) diluting a formulation of claim 81;
b) thawing and diluting a formulation of claim 82; and/or
c) resuspending and diluting a formulation of claim 83.

88. The method of claim 87, further comprising administering the dosage form to a subject in need of thereof.

89. The method of claim 88, wherein the subject is in need of an expression product of the mRNA.

90. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 5% w/v in the formulation;
c) trehalose at a concentration of about 5% w/v in the formulation;
d) His buffer, wherein the His buffer is substantially free of sodium chloride and is at a concentration of about 10 mM in the formulation;
wherein the formulation is diluted into the dosage form prior to administration.

91. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) HEPES buffer, wherein the HEPES buffer is substantially free of sodium chloride and is at a concentration of about 10 mM in the formulation.

92. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) HEPES buffer, wherein the HEPES buffer is substantially free of sodium chloride and is at a concentration of about 10 mM in the formulation.

93. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation before drying;
c) HEPES buffer, wherein the HEPES buffer is substantially free of sodium chloride and is at a concentration of about 10 mM in the formulation before drying.

94. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) HEPES buffer, wherein the HEPES buffer is substantially free of sodium chloride and is at a concentration of about 10 mM in the formulation; and
ii) sucrose at a concentration of about 10% w/v in the formulation.

95. The method of claim 94, further comprising a step of:
c) freezing the formulation.

96. The method of claim 94, further comprising a step of:
c) drying the formulation.

97. A method of preparing a dosage form, the method comprising a step of:
a) diluting a formulation of claim 91;
b) thawing and diluting a formulation of claim 92; and/or
c) resuspending and diluting a formulation of claim 93.

98. The method of claim 97, further comprising administering the dosage form to a subject in need of thereof.

99. The method of claim 98, wherein the subject is in need of an expression product of the mRNA.

100. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 10% w/v in the formulation;
d) HEPES buffer, wherein the HEPES buffer is substantially free of sodium chloride and is at a concentration of about 10 mM in the formulation;
wherein the formulation is diluted into the dosage form prior to administration.

101. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) HEPES buffer, wherein the HEPES buffer is substantially free of sodium chloride and is at a concentration of about 10 mM in the formulation.

102. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation;
c) HEPES buffer, wherein the HEPES buffer is substantially free of sodium chloride and is at a concentration of about 10 mM in the formulation.

103. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) trehalose at a concentration of about 10% w/v in the formulation before drying;
c) HEPES buffer, wherein the HEPES buffer is substantially free of sodium chloride and is at a concentration of about 10 mM in the formulation before drying.

104. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) HEPES buffer, wherein the HEPES buffer is substantially free of sodium chloride and is at a concentration of about 10 mM in the formulation; and
ii) trehalose at a concentration of about 10% w/v in the formulation.

105. The method of claim 104, further comprising a step of:
c) freezing the formulation.

106. The method of claim 104, further comprising a step of:
c) drying the formulation.

107. A method of preparing a dosage form, the method comprising a step of:
a) diluting a formulation of claim 101;
b) thawing and diluting a formulation of claim 102; and/or
c) resuspending and diluting a formulation of claim 103.

108. The method of claim 107, further comprising administering the dosage form to a subject in need of thereof.

109. The method of claim 108, wherein the subject is in need of an expression product of the mRNA.

110. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) trehalose at a concentration of about 10% w/v in the formulation;
d) HEPES buffer, wherein the HEPES buffer is substantially free of sodium chloride and is at a concentration of about 10 mM in the formulation;
wherein the formulation is diluted into the dosage form prior to administration.

111. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) HEPES buffer, wherein the HEPES buffer is substantially free of sodium chloride and is at a concentration of about 10 mM in the formulation.

112. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation;
c) trehalose at a concentration of about 5% w/v in the formulation;
d) HEPES buffer, wherein the HEPES buffer is substantially free of sodium chloride and is at a concentration of about 10 mM in the formulation.

113. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 5% w/v in the formulation before drying;
c) trehalose at a concentration of about 5% w/v in the formulation before drying;
d) HEPES buffer, wherein the HEPES buffer is substantially free of sodium chloride and is at a concentration of about 10 mM in the formulation before drying.

114. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) HEPES buffer, wherein the HEPES buffer is substantially free of sodium chloride and is at a concentration of about 10 mM in the formulation;
ii) sucrose at a concentration of about 5% w/v in the formulation; and
iii) trehalose at a concentration of about 5% w/v in the formulation.

115. The method of claim 114, further comprising a step of:
c) freezing the formulation.

116. The method of claim 114, further comprising a step of:
c) drying the formulation.

117. A method of preparing a dosage form, the method comprising a step of:
a) diluting a formulation of claim 111;
b) thawing and diluting a formulation of claim 12; and/or
c) resuspending and diluting a formulation of claim 113.

118. The method of claim 117, further comprising administering the dosage form to a subject in need of thereof.

119. The method of claim 118, wherein the subject is in need of an expression product of the mRNA.

120. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 5% w/v in the formulation;
c) trehalose at a concentration of about 5% w/v in the formulation;
d) HEPES buffer, wherein the HEPES buffer is substantially free of sodium chloride and is at a concentration of about 10 mM in the formulation;
wherein the formulation is diluted into the dosage form prior to administration.

121. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) PBS buffer, wherein the PBS buffer is substantially free of sodium chloride.

122. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) PBS buffer, wherein the PBS buffer is substantially free of sodium chloride.

123. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation before drying;
c) PBS buffer, wherein the PBS buffer is substantially free of sodium chloride.

124. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) PBS buffer, wherein the PBS buffer is substantially free of sodium chloride; and
ii) sucrose at a concentration of about 10% w/v in the formulation.

125. The method of claim 124, further comprising a step of:
c) freezing the formulation.

126. The method of claim 124, further comprising a step of:
c) drying the formulation.

127. A method of preparing a dosage form, the method comprising a step of:
a) diluting a formulation of claim 121;
b) thawing and diluting a formulation of claim 122; and/or
c) resuspending and diluting a formulation of claim 123.

128. The method of claim 127, further comprising administering the dosage form to a subject in need of thereof.

129. The method of claim 128, wherein the subject is in need of an expression product of the mRNA.

130. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 10% w/v;
c) PBS buffer, wherein the PBS buffer is substantially free of sodium chloride;
wherein the formulation is diluted into the dosage form prior to administration.

131. A formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) PBS buffer, wherein the PBS buffer comprises about 6 mg/ml sodium chloride in the formulation.

132. A frozen formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation;
c) PBS buffer, wherein the PBS buffer comprises about 6 mg/ml sodium chloride in the formulation.

133. A dry formulation comprising:
a) a lipid nanoparticle (LNP), wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) sucrose at a concentration of about 10% w/v in the formulation before drying;
c) PBS buffer, wherein the PBS buffer comprises about 6 mg/ml sodium chloride in the formulation before drying.

134. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) PBS buffer, wherein the PBS buffer comprises about 6 mg/ml sodium chloride in the formulation; and
ii) sucrose at a concentration of about 10% w/v in the formulation.

135. The method of claim 134, further comprising a step of:
c) freezing the formulation.

136. The method of claim 134, further comprising a step of:
c) drying the formulation.

137. A method of preparing a dosage form, the method comprising a step of:
a) diluting a formulation of claim 131;
b) thawing and diluting a formulation of claim 132; and/or
c) resuspending and diluting a formulation of claim 133.

138. The method of claim 137, further comprising administering the dosage form to a subject in need of thereof.

139. The method of claim 138, wherein the subject is in need of an expression product of the mRNA.

140. A method comprising a step of:
administering a dosage form of a formulation, wherein the formulation comprises:
a) a lipid nanoparticle (LNP), wherein the LNP comprises:
i) mRNA at a concentration of about 0.5 mg/ml;
ii) ((4-hydroxybutyl)azanediyl)bis(hexane-6,1-diyl)bis(2-hexyldecanoate) (ALC-0315) at a concentration of about 7.17 mg/ml;
iii) 2-[(polyethylene glycol)-2000]-N,N-ditetradecylacetamide (ALC-0159) at a concentration of about 0.89 mg/ml;
iv) distearoylphosphatidylcholine (DSPC) at a concentration of about 1.56 mg/ml;
v) cholesterol at a concentration of about 3.1 mg/ml;
b) sucrose at a concentration of about 10% w/v in the formulation;
c) PBS buffer, wherein the PBS buffer comprises about 6 mg/ml sodium chloride;
wherein the formulation is diluted into the dosage form prior to administration.

141. A method of preparing a formulation, the method comprising steps of:
a) preparing a lipid nanoparticle (LNP) in a first buffer system, wherein the LNP comprises:
i) a payload that is or comprises one or more mRNAs;
ii) lipids that include: ((4-hydroxybutyl)azanediyl)bis(hexane-6,1-diyl)bis(2-hexyldecanoate) (ALC-0315); 2-[(polyethylene glycol)-2000]-N,N-ditetradecylacetamide (ALC-0159); distearoylphosphatidylcholine (DSPC); and cholesterol at relative mass ratios in a range of about 8:1:1.5:3 to about 9:1:2:3.5; and
b) exchanging the first buffer system for a second buffer system, wherein the second buffer system comprises:
i) PBS buffer, wherein the PBS buffer comprises about 6 mg/ml sodium chloride in the formulation; and
ii) sucrose at a concentration of about 10% w/v in the formulation wherein the first buffer system comprises sucrose at a concentration of about 10% w/v.

142. The method of claim 141, further comprising a step of:
c) freezing the formulation.

143. The method of claim 141, further comprising a step of:
c) drying the formulation.

144. A method of delivering a nucleic acid into a cell in a subject comprising a step of administering a formulation as described in any of the preceding claims.

145. A method of inducing an immune response in a subject comprising a step of administering to the subject a formulation as described in any of the preceding claims.

146. The method of claim 145, wherein the immune response is against a viral antigen, or epitope thereof, encoded by the mRNA.

147. The method of claim 146, wherein the viral antigen is an antigen of a coronavirus.

148. The method of claim 147, wherein the coronavirus is a SARS-CoV2 virus.

149. The method of claim 147 or claim 148, wherein the antigen is or comprises an S protein.

150. The formulation of any one of claims 3, 13, 23, 33, 43, 53, 63, 73, 83, 93, 103, 113, 123, or 133, or the method of any one of claims 6, 16, 26, 36, 46, 56, 66, 76, 86, 96, 106, 116, 126, 136, or 143, wherein the formulation is dried until it is substantially free of water.

151. The formulation of any one of claims 3, 13, 23, 33, 43, 53, 63, 73, 83, 93, 103, 113, 123, 133, or 150, or the method of any one of claims 6, 16, 26, 36, 46, 56, 66, 76, 86, 96, 106, 116, 126, 136, 143, or 150, wherein the formulation is dried by lyophilization.

152. The formulation of any one of claims 3, 13, 23, 33, 43, 53, 63, 73, 83, 93, 103, 113, 123, 133, 150, or 151, or the method of any one of claims 6, 16, 26, 36, 46, 56, 66, 76, 86, 96, 106, 116, 126, 136, 143, 150, or 151, wherein the formulation is dried until it comprises less than 0.8%, less than 0.7%, less than 0.6%, less than 0.5%, less than 0.4%, or less than 0.3% w/w water.

153. The formulation of any one of claims 3, 13, 23, 33, 43, 53, 63, 73, 83, 93, 103, 113, 123, 133, 150, 151, or 152, or the method of any one of claims 6, 16, 26, 36, 46, 56, 66, 76, 86, 96, 106, 116, 126, 136, 143, 150, 151, or 152, wherein the formulation is annealed during drying.

154. The formulation of any one of claims 3, 13, 23, 33, 43, 53, 63, 73, 83, 93, 103, 113, 123, 133, 150, 151, or 152, or the method of any one of claims 6, 16, 26, 36, 46, 56, 66, 76, 86, 96, 106, 116, 126, 136, 143, 150, 151, or 152, wherein the formulation is not annealed during drying.

155. The formulation of any one of claims 150-154, or the method of any one of claims 150-154, wherein the formulation maintains less than 0.8%, less than 0.7%, less than 0.6%, less than 0.5%, less than 0.4%, or less than 0.3% w/w water for at least 12 weeks at temperatures ranging from about 2° C. to about 25° C.

156. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the one or more mRNAs encodes one or more polypeptides.

157. The formulation of claim 156, wherein the one or more polypeptides are or comprise an epitope for inducing an immune response against an antigen in a subject.

158. The formulation of any one of claims 1-3, 11-13, 21-23, 31-33, 41-43, 51-53, 61-63, 71-73, 81-83, 91-93, 101-103, 111-113, 121-123, or 131-133, wherein the one or more mRNAs are or comprise an epitope for inducing an immune response against an antigen in a subject.

159. The formulation of any one of claims 1-3, 11-13, 21-23, 31-33, 41-43, 51-53, 61-63, 71-73, 81-83, 91-93, 101-103, 111-113, 121-123, 131-133, or 158, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the one or more RNAs are or comprise self-amplifying RNA molecules.

160. The formulation of any one of claims 1-3, 11-13, 21-23, 31-33, 41-43, 51-53, 61-63, 71-73, 81-83, 91-93, 101-103, 111-113, 121-123, 131-133, 158, or 159, or the method of any one of claims 4-9, 14-19, 24-29, 34-39, 44-49, 54-59, 64-69, 74-79, 84-89, 94-99, 104-109, 114-119, 124-129, 134-139, 141-149, or 159, wherein the one or more RNAs are or comprise modified RNA molecules, or non-modified RNA molecules.

161. The formulation of any one of claims 1-3, 11-13, 21-23, 31-33, 41-43, 51-53, 61-63, 71-73, 81-83, 91-93, 101-103, 111-113, 121-123, 131-133, or 158-160, or the method of any one of claims 4-9, 14-19, 24-29, 34-39, 44-49, 54-59, 64-69, 74-79, 84-89, 94-99, 104-109, 114-119, 124-129, 134-139, 141-149, 159, or 160, wherein the one or more RNAs are or comprise non-modified uridine RNA molecules.

162. The formulation of any one of claims 1-3, 11-13, 21-23, 31-33, 41-43, 51-53, 61-63, 71-73, 81-83, 91-93, 101-103, 111-113, 121-123, 131-133, or 158-161, or the method of any one of claims 4-9, 14-19, 24-29, 34-39, 44-49, 54-59, 64-69, 74-79, 84-89, 94-99, 104-109, 114-119, 124-129, 134-139, 141-149, or 159-161, wherein the one or more RNAs are or comprise nucleoside modified RNA molecules.

163. The formulation of claim 156, or the method of claim 156, wherein at least one of the one or more polypeptides is derived from a SARS-COV-2 S Protein of SEQ ID NO: 1 or 7.

164. The formulation of claim 156, or the method of claim 156, wherein at least one of the one or more polypeptides comprises at least 85% sequence identity to the SARS-COV-2 S Protein of SEQ ID NO: 1 or 7.

165. The formulation of claim 156, or the method of claim 156, wherein at least one of the one or more polypeptides is or comprises a full length SARS-COV-2 S Protein of SEQ ID NO: 1 or 7.

166. The formulation of claim 156, or the method of claim 156, wherein at least one of the one or more polypeptides comprises at least 85% sequence identity to the Receptor Binding Domain (RBD) of SARS-COV-2 S Protein of SEQ ID NO: 1 or 7.

167. The formulation of claim 156, or the method of claim 156, wherein at least one of the one or more polypeptides is or comprises a Receptor Binding Domain (RBD) of SARS-COV-2 S Protein of SEQ ID NO: 1 or 7.

168. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the one or more mRNAs are associated or encapsulated in the LNP.

169. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation has been stored over a time period of at least 12 weeks at a temperature above 25° C.

170. The of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation has been stored over a time period of at least 12 weeks at a temperature in a range of about 2-40° C.

171. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized in that lipid nanoparticles exhibit less than 20 nm change in Z-Average when stored over a time period of at least 12 weeks at a temperature of about 2-8° C. as compared to a control formulation.

172. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized in that lipid nanoparticles exhibit less than 20 nm change in Z-Average when stored over a time period of at least 12 weeks at a temperature of about 25° C. as compared to a control formulation.

173. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized in that lipid nanoparticles exhibit less than 0.1 change in Polydispersity Index (PDI) when stored over a time period of at least 12 weeks at a temperature of about 2-8° C. as compared to a control formulation.

174. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized in that lipid nanoparticles exhibit less than 0.1 change in Polydispersity Index (PDI) when stored over a time period of at least 12 weeks at a temperature of about 25° C. as compared to a control formulation.

175. The formulation of any one of claims 1-3, 11-13, 21-23, 31-33, 4143, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized by a % mRNA encapsulation of at least 40%, at least 50%, at least 60%, at least 70%, at least 80%, or at least 90% when stored over a time period of at least 12 weeks at a temperature of about 2-8° C. as compared to a control formulation.

176. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized by mRNA encapsulation % of at least 40%, at least 50%, at least 60%, at least 70%, at least 80%, or at least 90% when stored over a time period of at least 12 weeks at a temperature of about 25° C. as compared to a control formulation.

177. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized by a % mRNA expression of at least 60%, at least 70%, at least 80%, or at least 90% when stored over a time period of at least 12 weeks at a temperature of about 2-8° C. as compared to a control formulation.

178. The formulation of any one of claims 1-3, 11-13, 21-23, 31-33, 41-43, 51-53, 61-63, 71-73, 81-83, 91-93, 101-103, 111-113, 121-123, or 131-133, or the method of any one of claims 4-9, 14-19, 24-29, 34-39, 44-49, 54-59, 64-69, 74-79, 84-89, 94-99, 104-109, 114-119, 124-129, 134-139, or 141-149, wherein the formulation is characterized by a % mRNA expression of at least 60%, at least 70%, at least 80%, or at least 90% when stored over a time period of at least 12 weeks at a temperature of about 25° C. as compared to a control formulation.


SEQUENCE LISTING (20 sequences)

SEQ ID NO: 1 - protein, 1273 aa, Artificial Sequence
  SARS-CoV-2 spike (S) protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 3819 nt, Artificial Sequence
  Coding Sequence
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauaaaguggaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 3-6 - skipped (4 entries)

SEQ ID NO: 7 - protein, 1273 aa, Artificial Sequence
  SARS-CoV-2 spike (S) protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - RNA, 3819 nt, Artificial Sequence
  Coding Sequence
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 9 - RNA, 3819 nt, Artificial Sequence
  Coding Sequence
auguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacaca

SEQ ID NO: 10-18 - skipped (9 entries)

SEQ ID NO: 19 - RNA, 4283 nt, Artificial Sequence
  RBP020.1
agaauaaacuaguauucuucugguccccacagacucagagagaacccgccaccauguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 20 - RNA, 4283 nt, Artificial Sequence
  RBP020.2
agaauaaacuaguauucuucugguccccacagacucagagagaacccgccaccauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa